(19) United States
(12) Patent Application Publication
(10) Pub. No.: US 2025/0066759 A1
(43) Pub. Date: Feb. 27, 2025

(54) FUSION PROTEINS FOR PURIFYING NUCLEIC ACIDS

(71) Applicant: Donaldson Company, Inc., Bloomington, MN (US)

(72) Inventors: Michael DZURICKY, Durham, NC (US); Kelli M. LUGINBUHL, Durham, NC (US); James MCGUINESS, Bloomington, MN (US)

(21) Appl. No.: 18/813,397

(22) Filed: Aug. 23, 2024

Related U.S. Application Data
(60) Provisional application No. 63/554,715, filed on Feb. 16, 2024.
(60) Provisional application No. 63/578,551, filed on Aug. 24, 2023.

(51) Int. Cl. C12N 15/10; C07K 14/32

(52) U.S. Cl. CPC C12N 15/101; C07K 14/32; C07K 2319/85

(57) ABSTRACT

Provided herein are fusion proteins and methods of use thereof to purify nucleic acids from a composition. The disclosed compositions and methods allow for faster, more efficient purification of nucleic acids compared to traditional affinity chromatography.

### CROSS REFERENCE TO RELATED APPLICATIONS

This application claims priority to U.S. Application No. 63/578,551 filed on Aug. 24, 2023, and U.S. Application No. 63/554,715 filed on Feb. 16, 2024, the contents of which is incorporated by reference herein in its entirety for all purposes.

### REFERENCE TO AN ELECTRONIC SEQUENCE LISTING

The contents of the electronic sequence listing (ISOL_011_02US_SeqList_ST26.xml; Size: 1,566,780 bytes; and Date of Creation: Aug. 21, 2024) are herein incorporated by reference in its entirety.

### FIELD

The present disclosure is generally related to fusion proteins, compositions containing fusion proteins, and methods of using fusion proteins for purification of nucleic acids.

### BACKGROUND

The use of nucleic acids as therapeutics is rapidly increasing, as highlighted by the mRNA-based vaccines against SARS-CoV-2. Purification of nucleic acids like mRNA is challenging because mRNAs are large molecules, much bigger than proteins. As a result of its large size, mRNA does not interact well with convention affinity chromatography resins. Additionally, the impurity profile of nucleic acid compositions is often complex and it is difficult to find a purification platform that can remove all contaminants from the compositions.

There is a need in the art for improved compositions and methods for rapidly and cost-effectively purifying nucleic acids like mRNA.

### SUMMARY

The present disclosure provides fusion proteins containing nucleic acid binding proteins and methods of using the same for purifying nucleic acids, and methods for using the same.

Provided herein are compositions comprising a fusion protein. In embodiments, the fusion protein comprises a nucleic acid binding protein (NBP) and a polypeptide with phase behavior. In embodiments, the NBP is selected from any one of: RNA-specific adenosine deaminase 1 (ADAR1), ADAR1 double stranded RNA-binding domain 3 (dsRBD3), Bacillus subtilis cold shock protein B (Bs-CspB), cold shock domain Y-box protein (CSD-Ybox), eukaryotic translation initiation factor 4E (eIF4e), Fox-1 protein (FOX1), heterogenous nuclear ribonucleoprotein Q1 (hnRNPQ1), Homo sapiens zinc finger CCCH-Type containing 14 (HsZC3H14), polyA-binding protein (PABP), polyA-binding protein nuclear 1 (PABPN1), pentatricopeptide repeat protein A (PPRpA), Pumilio-like repeat protein A (PUFpA), Staufen, 12-O-tetradecanoylphorbol-13-acetate inducible sequence 11 D (TIS11D), Z-DNA/RNA binding protein 1 (ZBP1), and Zinc Finger Nuclease (ZNF). In embodiments, the NBP has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99 or 100% identity to a polypeptide of any one of SEQ ID NOS: 61-103. In embodiments, the NBP is selected from any one of: ADAR1 double stranded RNA-binding domain 3 (dsRBD3), heterogenous nuclear ribonucleoprotein Q1 (hnRNPQ1), Homo sapiens zinc finger CCCH-Type containing 14 (HsZC3H14), polyA-binding protein nuclear 1 (PABPN1), pentatricopeptide repeat protein A (PPRpA), and Pumilio-like repeat protein A (PUFpA). In embodiments, the NBP has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 89, 93, 95, or 97-99. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-60 or 217. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 57 or 60.

In embodiments, the fusion protein comprises a linker. In embodiments, the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 143-216.

In embodiments, the NBP binds to RNA, DNA, or both. In embodiments, the NBP binds to RNA selected from any one of double stranded RNA (dsRNA), single stranded RNA (ssRNA), mRNA, pre-mRNA, polyadenosine (polyA) RNA, Z-confirmation RNA (Z-RNA), or a combination thereof. In embodiments, the NBP binds to the 3′-terminus of mRNA, the 3′-terminus of mRNA, a 3′ untranslated region (UTR) of mRNA, a polyA tail of mRNA, or an AU-rich element, or combination thereof, of mRNA. In embodiments, the NBP binds to a pre-mRNA. In embodiments, the NBP binds to an intron, exon, polyA tail, or combination thereof, of the pre-mRNA. In embodiments, the NBP binds to DNA. In embodiments, the NBP binds to single stranded DNA, double stranded DNA, polyadenosine (polyA) DNA, Z-conformation DNA (Z-DNA), or a combination thereof.

In embodiments, the fusion protein is encoded by a nucleic acid. In embodiments, the nucleic acid has a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 119-133. In embodiments, the fusion protein is encoded by a vector. In embodiments, the vector comprises a nucleic acid sequence, wherein the nucleic acid has a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 119-133. In embodiments, the vector has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 104-118, 134, and 135.

Provided herein are methods of purifying a nucleic acid, comprising (i) contacting a composition comprising the nucleic acid and at least one contaminant with a fusion protein of the disclosure, wherein the fusion protein binds to the nucleic acid to form a complex; (ii) contacting the complex with a first environmental factor to increase the size of the complex; (iii) separating the complex from at least one contaminant; and (iv) separating the nucleic acid from the fusion protein by contacting the complex with a second environmental factor. In embodiments, the complex is separated from the at least one contaminant on the basis of size. In embodiments, the separation on the basis of size is performed using any one of the methods selected from tangential flow filtration, membrane chromatography, analytical ultracentrifugation, high performance liquid chromatography, membrane chromatography, normal flow filtration, acoustic wave separation, centrifugation, counterflow centrifugation, and fast protein liquid chromatography. In embodiments, the first environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves. In embodiments, the second environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves. In embodiments, the at least one contaminant is selected from a solvent, a protein, a peptide, a carbohydrate, a nucleic acid, a virus, a cell (e.g., a bacterial, yeast, or mammalian cell), a carbohydrate, a lipid, or a lipopolysaccharide.

Provided herein is a fusion protein comprising a Bacillus subtilis cold shock protein B (Bs-CspB) and a polypeptide with phase behavior. In embodiments, the Bs-CspB has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 90. In embodiments, the polypeptide with phase behavior comprises a P and G motif, comprising a plurality of proline residues and a plurality of glycine residues. In embodiments, the P and G motif comprises at least about 10% proline residues and at least about 20% glycine residues. In embodiments, the polypeptide with phase behavior comprises pentapeptide repeat having the sequence (Val-Pro-Gly-Xaa-Gly)n (SEQ ID NO: 217), or a randomized, scrambled analog thereof; wherein Xaa can be any amino acid except proline. In embodiments, n is an integer from 1 to 360, inclusive of endpoints. In embodiments, the polypeptide with phase behavior comprises an amino acid sequence selected from: a. (GRGDSPY)n (SEQ ID NO: 1); b. (GRGDSPH)n (SEQ ID NO: 2); c. (GRGDSPV)n (SEQ ID NO: 3); d. (GRGDSPYG)n (SEQ ID NO: 4); e. (RPLGYDS)n (SEQ ID NO: 5); f. (RPAGYDS)n (SEQ ID NO: 6); g. (GRGDSYP)n (SEQ ID NO: 7); h. (GRGDSPYQ)n (SEQ ID NO: 8); i. (GRGNSPYG)n (SEQ ID NO: 9); j. (GVGVP)n (SEQ ID NO: 10); k. (GVGVPGLGVPGVGVPGLGVPGVGVP)m (SEQ ID NO: 11); l. (GVGVPGVGVPGAGVPGVGVPGVGVP)m (SEQ ID NO: 12); m. (GVGVPGWGVPGVGVPGWGVPGVGVP)m (SEQ ID NO: 13); n. (GVGVPGVGVPGVGVPGVGVPGVGVPGVGVPGEGVPGFGVPGVGVP)m (SEQ ID NO: 14); o. (GVGVPGVGVPGVGVPGVGVPGVGVPGVGVPGKGVPGFGVPGVGVP)m (SEQ ID NO: 15); and p. (GAGVPGVGVPGAGVPGVGVPGAGVP)m (SEQ ID NO: 16); or a randomized, scrambled analog thereof; wherein: n is an integer in the range of 20-360, inclusive of endpoints; and m is an integer in the range of 4-25, inclusive of endpoints. In embodiments, the polypeptide with phase behavior comprises an amino acid sequence selected from: a. (GVGVP)m (SEQ ID NO: 22); b. (ZZPXXXXGZ)m (SEQ ID NO: 23); c. (ZZPXGZ)m (SEQ ID NO: 24); d. (ZZPXXGZ)m (SEQ ID NO: 25); or e. (ZZPXXXGZ)m (SEQ ID NO: 26), wherein m is an integer between 10 and 160, inclusive of endpoints, wherein X if present is any amino acid except proline or glycine, and wherein Z if present is any amino acid.

In embodiments, the polypeptide with phase behavior comprises an amino acid sequence selected from: a. (GVGVPGVGVPGAGVPGVGVPGVGVP)m (SEQ ID NO: 17); or b. (GVGVPGVGVPGLGVPGVGVPGVGVP)m (SEQ ID NO: 18); wherein m is an integer between 2 and 32, inclusive of endpoints. In embodiments, the polypeptide with phase behavior comprises an amino acid sequence selected from: a. (GVGVPGVGVPGAGVPGVGVPGVGVP)m (SEQ ID NO: 19), wherein m is 8 or 16; b. (GVGVPGAGVP)m (SEQ ID NO: 20), wherein m is an integer between 5 and 80, inclusive of endpoints; or c. (GXGVP)m (SEQ ID NO: 21), wherein m is an integer between 10 and 160, inclusive of endpoints, and wherein X for each repeat is independently selected from the group consisting of glycine, alanine, valine, isoleucine, leucine, phenylalanine, tyrosine, tryptophan, lysine, arginine, aspartic acid, glutamic acid, and serine. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-60, 217, or 262-264. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 56, or 262-264. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 56. In embodiments, the fusion protein has at least 90% identity to a polypeptide of SEQ ID NO: 90 and at least 90% identity to a polypeptide of any one of SEQ ID NOs: 56, and 262-264. In embodiments, the fusion protein has at least 90% identity to a polypeptide of SEQ ID NO: 90 and at least 90% identity to a polypeptide of SEQ ID NO: 56.

In embodiments, the fusion protein comprising a linker. In embodiments, the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 143-216, or 261. In embodiments, the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 261. In embodiments, the fusion protein has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 225 or 226.

In embodiments, the fusion protein has at least 90% identity to a polypeptide of SEQ ID NO: 225 or 226. In embodiments, the Bs-CspB binds to RNA, DNA, or both. In embodiments, the Bs-CspB binds to RNA, DNA, or both. In embodiments, the Bs-CspB binds to ssRNA. In embodiments, the Bs-CspB binds to a 3′-terminus of the mRNA, a 5′-terminus of the mRNA, a coding region of the mRNA, a non-coding region of the mRNA, or a combination thereof. In embodiments, the Bs-CspB binds to a pre-mRNA. In embodiments, the Bs-CspB binds to an intron, an exon, a 5′ UTR, a 3′ UTR, or a combination thereof, of the pre-mRNA. In embodiments, the Bs-CspB binds to DNA. In embodiments, the Bs-CspB binds to single stranded DNA, double stranded DNA, polyadenosine (polyA) DNA, Z-conformation DNA (Z-DNA), or a combination thereof.

Provided herein is a nucleic acid encoding the fusion protein. In embodiments, the nucleic acid has a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 119-133.

Provided herein is a vector encoding a fusion protein. Provided herein is a vector comprising the nucleic acid. Provided herein is a vector having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid any one of SEQ ID NOS: 104-118, 134, and 135.

Provided herein is a method of purifying a single stranded nucleic acid comprising: (i) contacting a composition comprising the single stranded nucleic acid and at least one contaminant with a fusion protein of any one of claims 1-28, wherein the fusion protein binds to the single stranded nucleic acid to form a complex; (ii) adding to the composition comprising the complex a first environmental factor, thereby increasing the size of the complex; (iii) separating the complex from at least one contaminant; and (iv) separating the single stranded nucleic acid from the fusion protein by contacting the complex with a second environmental factor, thereby forming a product comprising the single stranded nucleic acid. In embodiments, the single stranded nucleic acid comprises ssRNA. In embodiments, the complex is separated from the at least one contaminant on the basis of size. In embodiments, the separation on the basis of size is performed using a method selected from any one of tangential flow filtration, membrane chromatography, analytical ultracentrifugation, high performance liquid chromatography, normal flow filtration, acoustic wave separation, centrifugation, counterflow centrifugation, and fast protein liquid chromatography. In embodiments, the separation on the basis of size is performed using a method comprising centrifugation. In embodiments, the first environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves. In embodiments, the second environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves. In embodiments, the at least one contaminant is selected from a solvent, a protein, a peptide, a carbohydrate, double stranded nucleic acid, a virus, a cell (e.g., a bacterial, yeast, or mammalian cell), a carbohydrate, a lipid, or a lipopolysaccharide. In embodiments, the at least one contaminant is a double stranded nucleic acid. In embodiments, the double stranded nucleic acid is dsRNA. In embodiments, the salt is added at a concentration comprising about 0.5 M to about 3 M. In embodiments, the salt is added at a concentration comprising about 1.2 M to about 1.7 M. In embodiments, the product comprises about 70% to about 100% of the single stranded nucleic acid based on the total nucleic acid content in the product of step (iv). In embodiments, the single stranded nucleic acid comprises ssRNA. In embodiments, the product comprises about 10% or less of the at least one contaminant based on the total nucleic acid content in the product. In embodiments, the at least one contaminant comprises dsRNA. In embodiments, the purification method comprises at least about a 1-log removal of a contaminant compared to the amount of the contaminant in the composition of step (i). In embodiments, the purification method comprises a 1 to 10-log removal of a contaminant compared to the amount of the contaminant in the composition of step (i). In embodiments, in step (i), the fusion protein is present at a concentration of about 1 μM to about 200 μM. In embodiments, in step (i), the fusion protein is present at a concentration of about 30 μM to about 60 μM.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 shows a comparison of non-specific binding among various polypeptides with phase behavior to RNA templates of varying length and strandedness and linearized plasmid DNA using agarose gel electrophoresis. Successful binding of the nucleic acid species by the biopolymer is indicated as retention within the well. The polypeptide with phase behavior 40L80 (SEQ ID NO: 264) demonstrated non-specific binding to a broad range of nucleic acid species.

FIG. 2 shows the percent elution of total RNA captured directly using an ssRNA purification reagent comprising a fusion protein (SEQ ID NO: 225) containing Bs-CspB (SEQ ID NO: 90) and a polypeptide with phase behavior, referred to as 100V80 (SEQ ID NO: 56), from in vitro transcription (IVT) reactions separately transcribing a range of mRNA template sizes. The percent elution reflects the final yield post-elution compared to total RNA present in the initial IVT reaction.

FIGS. 3A and 3B shows that the ssRNA purification reagent comprising Bs-CspB and 100V80 is highly selective for ssRNA. FIG. 3A shows the Log₁₀ removal value (LRV) of dsRNA during RNA captures of purified RNA with spiked in dsRNA across a range of ssRNA purification reagent:RNA molar ratios. The reagent retained selectivity for ssRNA and was capable of greater than 4-log removal of dsRNA. FIG. 3B shows the percent elution of total dsRNA across samples with a range of percent spiked dsRNA. Despite spiking in dsRNA at values as high as 10% of the total RNA, selectivity was retained in captures performed on purified RNA.

FIG. 4 shows a screen of ssRNA binding candidates for their ability to capture a broad range of ssRNA templates and dsRNA targets in independent capture reactions using agarose gel electrophoresis. The fusion protein comprising Bs-CspB and 100V80 selectively captured a board range of ssRNA templates. The presence of a band indicates uncaptured nucleic acids.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Moreover, the present disclosure also contemplates that in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

As used herein, the term “environmental factor” is any factor that, when applied to a composition comprising a fusion protein, alters one or more properties of the composition. Non-limiting examples of environmental factors include a change in one or more of temperature, pH, salt concentration, concentration of the fusion protein, concentration of the nucleic acid, or pressure; the addition of one or more surfactants, molecular crowding agents, denaturing agents, reducing agents, or oxidizing agents; or the application of electromagnetic or acoustic waves.

As used herein, the term “contaminant” may refer to any substance that is not desired in a purified composition. In embodiments, the contaminant is any substance other than the nucleic acid desired to be purified. Non-limiting examples of contaminants include, but are not limited to, a solvent, a protein, a peptide, a carbohydrate, a nucleic acid, a virus, a cell (e.g., a bacterial, yeast, or mammalian cell), a carbohydrate, a lipid, or a lipopolysaccharide. In embodiments, the contaminant is an endotoxin or a mycotoxin. In embodiments, a the cell is an immune cell. In embodiments, the immune cell is a T cell, a B cell, a NK cell, a peripheral blood mononuclear cell, monocyte, macrophage, or a neutrophil. In embodiments, the cell is a T cell expressing a chimeric antigen receptor (CAR). In embodiment, the contaminant is double stranded nucleic acid.

As used herein, the terms “peptide,” “polypeptide,” and “protein” are used interchangeably, and refer to a compound comprised of amino acid residues covalently linked by peptide bonds. A protein must contain at least two amino acids, and no limitation is placed on the maximum number of amino acids that can comprise a protein's sequence. The term “peptide” may refer to a short chain of amino acids including, for example, natural peptides, recombinant peptides, synthetic peptides, or a combination thereof. Proteins and peptides may include, for example, biologically active fragments, substantially homologous polypeptides, oligopeptides, homodimers, heterodimers, variants of polypeptides, modified polypeptides, derivatives, analogs, and fusion proteins, among others.

A “polynucleotide” is a sequence of nucleotide bases, and may be RNA, DNA or DNA-RNA hybrid sequences (including both naturally occurring and/or non-naturally occurring nucleotides). In embodiments, a polynucleotide is either a single or double stranded DNA sequence.

As used herein, by “isolate” or “purify” (or grammatical equivalents) a nucleic acid, it is meant that the nucleic acid is at least partially separated from at least some of the other components in a starting material comprising the nucleic acid (e.g., a cell lysate). In embodiments, an “isolated” or “purified” nucleic acid may be enriched by at least about 10-fold, about 100-fold, about 1000-fold, about 10,000-fold or more as compared with the starting material.

As used herein, the term “polypeptide with phase behavior” refers to any polypeptide that is capable of undergoing a phase transition. In embodiments, the polypeptide undergoes a phase transition due to the application of an environmental factor. Exemplary polypeptides with phase behavior include elastin-like polypeptides (ELPs) and resilin-like polypeptides (RLPs).

As used herein, the term “fusion protein” refers to a polypeptide produced when two heterologous nucleotide sequences or fragments thereof coding for two (or more) different polypeptides not found fused together in nature are fused together in the correct translational reading frame.

As used herein, the term “nucleic acid binding protein”, also known as an NBP, may refer to any amino acid sequence (protein, peptide, etc.) which binds to a target nucleic acid. In embodiments, the NBP may comprise a full-length, truncated, or modified version of a receptor for the target nucleic acid. In embodiments, the NBP may be an antigen-binding portion of a monoclonal antibody (e.g., a Fab), a single-chain variable fragment (scFv) derived from a monoclonal antibody; a natural ligand of the target nucleic acid; a peptide with sufficient affinity for the target nucleic acid; a single domain binder such as a camelid; an artificial binder such as a Darpin; or a single-chain derived from a T-cell receptor.

As used herein, the term “fragment” as it refers to a protein or polypeptide includes a truncated form of the protein or polypeptide. For example, a fragment of an NBP may include from about 50% to about 99.9% of the full length NBP. In embodiments, the fragment of the NBP comprises about 50%, about 55%, about 60%, about 65%, about 70%, about 75%, about 80%, about 85%, about 90%, about 95%, about 97%, or about 99% of the amino acids of the full length NBP.

As used herein, the term “capture efficiency” as it relates to a fusion protein described herein refers to the amount of nucleic acid captured by a fusion protein relative to the amount of nucleic acid present in the starting composition. The following equation is used to determine capture efficiency: 100×(amount of nucleic acid captured by the fusion protein/amount of nucleic acid in the composition before purification).

The term “percent identity” in the context of two or more nucleic acid or polypeptide sequences, refers to two or more sequences or subsequences that have a specified percentage of nucleotides or amino acid residues that are the same, when compared. Unless otherwise indicated, sequence identity is determined using the National Center for Biotechnology Information (NCBI)'s Basic Local Alignment Search Tool (BLAST®), available at blast.ncbi.nlm.nih.gov/Blast.cgi. In embodiments, the sequence identity is calculated over the entire length of the compared sequences. In embodiments, the sequence identity is calculated over a fragment of each compared sequence of about a 20-amino acid, 50-amino acid, 75-amino acid, 100-amino acid, 250-amino acid, 500-amino acid, 750-amino acid, or 1000-amino acids.

### Fusion Proteins

The disclosure provides fusion proteins and methods of using the fusion proteins to purify nucleic acids. In embodiments, the fusion protein comprises a NBP which binds to the target nucleic acid (i.e., a nucleic acid binding protein) and a polypeptide with phase behavior, wherein the NBP is coupled to the polypeptide with phase behavior.

### Nucleic Acid Binding Proteins

In embodiments, the fusion protein comprises a nucleic acid binding protein (NBP). In embodiments, the NBP binds to one or more nucleic acids. In embodiments, the NBP binds to a single stranded nucleic acid (e.g., single stranded DNA or RNA). In embodiments, the NBP binds to a double stranded nucleic acid (e.g., double stranded DNA or RNA). In embodiments, the NBP binds to DNA. In embodiments, the DNA is single stranded or double stranded. In embodiments, the NBP binds to RNA. In embodiments, the RNA is single stranded or double stranded. In embodiments, the NBP binds to both DNA and RNA, wherein the DNA and RNA are single stranded, double stranded, or a combination of both. In embodiments, the NBP binds to polyadenosine (polyA) DNA. In embodiments, the NBP binds to polyadenosine (polyA) RNA. In embodiments, the NBP binds to polythymidine (polyT) DNA. In embodiments, the NBP binds to polyuridine (PolyU) RNA. In embodiments, the NBP binds to polycytidine (polyC) RNA. In embodiments, the NBP binds to polyguanidine (polyG) RNA. In embodiments, the NBP binds to polycytidine (polyC) DNA. In embodiments, the NBP binds to polyguanidine (polyG) DNA.

In embodiments, the NBP binds to DNA, a microRNA, capped RNA, DNA, double stranded RNA, transfer RNA, ribosomal RNA, a small nuclear RNA, a regulatory RNA, a ribozyme, a transfer RNA, or a messenger RNA.

In embodiments, the NBP binds to an AU-rich RNA element (ARE). An ARE refers to an adenylate-uridylate-rich element in the 5′ or 3′ untranslated region of a mRNA. AREs contain the core sequence AUUUA. AREs are a determinant of RNA stability, and often occur in mRNAs of proto-oncogenes, nuclear transcription factors, and cytokines. Proteins that bind to ARE are referred to as ARE-binding proteins (ARE-BP). In embodiments, ARE-BP stabilize mRNA. Non-limiting examples of ARE-BP include human antigen R (huR, also called “ELAV”), tristetrapolin (TTP), AU-rich element RNA-binding protein (AUF), and fragile X mental retardation syndrome-related protein 1 (FXR1). The following articles describe ARE-BP and are incorporated by reference herein in their entirety: Otsuka et al. Front. Genet., 2 May 2019; Brennan and Steitz. Cell Mol Life Sci. 2001 February; 58(2):266-77; Carballo et al. 1998. Science, 281, 1001-1005; Mazan-Mamczarz et al. Oncogene volume 27, pages 6151-6163 (2008); Vasudevan and Steitz. Cell. 2007 Mar. 23; 128(6):1105-18; Curr Cancer Drug Targets. 2019; 19(5):382-399; J Biol Chem. 2017 Apr. 28; 292(17):6869-6881. doi: 10.1074/jbc.M116.772947. Epub 2017 Mar. 16; Wiley Interdiscip Rev RNA. July-August 2014; 5(4):549-64. doi: 10.1002/wrna.1230; and Elife. 2017 Aug. 2; 6:e26129. doi: 10.7554/eLife.26129; Mazan-Mamczarz et al. 2008. Nucleic Acids Research, 37, 204-214. In embodiments, an NBP binds to an ARE. In embodiments, the NBP binding to an ARE incorporates a binding element of huR, TTP, AUF, or FXR1. AREs are disclosed in the reference incorporated in its entirety herewith: Barreau C, et al. AU-rich elements and associated factors: are there unifying principles? Nucleic Acids Res. 2006 Jan. 3; 33(22):7138-50. In embodiments, the NBP binds to Z-conformation DNA (Z-DNA).

In embodiments, the NBP binds to Z-conformation RNA (Z-RNA). In general, Z-DNA and R-DNA are left-handed structures of DNA and RNA, respectively as described in the reference incorporated in its entirety herein: Barreau C, et al. AU-rich elements and associated factors: are there unifying principles? Nucleic Acids Res. 2006 Jan. 3; 33(22):7138-50.

In embodiments, the NBP binds to microRNA.

In embodiments, the NBP binds to small nuclear RNA (snRNA). In embodiments, the NBP binds to small nucleolar RNA (snoRNA).

In embodiments, the NBP binds to regulatory RNA.

In embodiments, the NBP binds to a ribozyme.

In embodiments, the NBP binds to transfer RNA (tRNA). In embodiments, the NBP binds to long non-coding RNA (lncRNA).

In embodiments, the NBP binds to mRNA. In embodiments, the NBP binds to pre-mRNA. In embodiments, the NBP binds to a pre-mRNA intron. In embodiments, the NBP binds to a pre-mRNA exon. In embodiments, the NBP binds to the 3′ termini of mRNA. In embodiments, the NBP binds to 5′ termini of mRNA. In embodiments, the NBP binds to 5′ cap of mRNA. In embodiments, the NBP binds to a positively charged, intrinsically disorder region (IDR) or a nucleic acid. In embodiments, the NBP binds to a nucleic acid sequence. In embodiments, the NBP binds to a nucleic acid structure. In embodiments, the NBP binds to a nucleic acid secondary structure. In embodiments, the NBP binds to a nucleic acid tertiary structure. In embodiments, the NBP binds to a naturally occurring nucleic acid. In embodiments, the NBP binds to a synthetically made nucleic acid. In embodiments, the nucleic acid is naturally occurring but has been modified by synthetic means.

In embodiments, the NBP binds to a nucleic acid sequence, wherein the nucleic acid sequence is about 30 nucleotides to about 10,000 nucleotides in length. In embodiments, the nucleic acid is at least about 30 nucleotides in length. In embodiments, the nucleic acid is at least about 35 nucleotides in length, such as at least about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 4,000, 5,000, 6,000, 7,000, 8,000, 9,000, or at least about 10,000 nucleotides in length. In embodiments, the nucleic acid is at least about 100 nucleotides in length. In embodiments, the nucleic acid is at least about 250 nucleotides in length. In embodiments, the nucleic acid is at least about 500 nucleotides in length. In embodiments, the nucleic acid is at least about 1,000 nucleotides in length. In embodiments, the nucleic acid is at least about 1,500 nucleotides in length. In embodiments, the nucleic acid is at least about 2,000 nucleotides in length. In embodiments, the nucleic acid is at least about 2,500 nucleotides in length. In embodiments, the nucleic acid is at least about 3,000 nucleotides in length. In embodiments, the nucleic acid is at least about 4,000 nucleotides in length. In embodiments, the nucleic acid is at least about 5,000 nucleotides in length. In embodiments, the nucleic acid is at least about 6,000 nucleotides in length. In embodiments, the nucleic acid is at least about 7,000 nucleotides in length. In embodiments, the nucleic acid is at least about 8,000 nucleotides in length. In embodiments, the nucleic acid is at least about 9,000 nucleotides in length. In embodiments, the nucleic acid is at least about 10,000 nucleotides in length.

In embodiments, the nucleic acid has a size from about 0.001 μm to about 500 μm in diameter or length. In embodiments, the nucleic acid has a diameter between 1 nm and 100 μm, inclusive of the endpoints. In embodiments, the nucleic acid has a diameter between 1 nm and 100 nm, inclusive of the endpoints. In embodiments, the nucleic acid has a diameter between 100 nm and 1 μm, inclusive of the endpoints. In embodiments, the nucleic acid has a diameter between 1 μm and 50 μm, inclusive of the endpoints. In embodiments, the nucleic acid has a diameter between 50 μm and 100 μm, inclusive of the endpoints.

In embodiments, the size (i.e., diameter or length) of the nucleic acid is about 0.001 μm, about 0.002 μm, about 0.003 μm, about 0.004 μm, about 0.005 μm, about 0.006 μm, about 0.007 μm, about 0.008 μm, about 0.009 μm, about 0.010 μm, about 0.020 μm, about 0.030 μm, about 0.040 μm, about 0.050 μm, about 0.060 μm, about 0.070 μm, about 0.080 μm, about 0.090 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 lam, about 6 lam, about 7 lam, about 8 lam, about 9 lam, about 10 lam, about 11 μm, about 12 lam, about 13 μm, about 14 μm, about 15 μm, about 16 μm, about 17 μm, about 18 μm, about 19 μm, about 20 lam, about 21 μm, about 22 lam, about 22 lam, about 23 lam, about 24 lam, about 25 lam, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, or about 500 μm, or greater, including all values and ranges in between. In embodiments, the nucleic acid has a size of greater than or equal to 10 μm. In embodiments, the nucleic acid has a size that is greater than or equal to 25 μm. In embodiments, the nucleic acid has a size that is greater than or equal to 50 μm. In embodiments, the nucleic acid has a size that is greater than or equal to 100 μm.

In some embodiments, the nucleic acid has a size (i.e., molar mass) from about 2 kDa to about 1000 MDa. In embodiments, the nucleic acid has a molar mass of about 2 kDa, about 5 kDa, about 15 kDa, about 20 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 150 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1 MDa, about 5 MDa, about 10 MDa, about 15 MDa, about 20 MDa, about 25 MDa, about 50 MDa, about 75 MDa, about 100 MDa, about 125 MDa, about 150 MDa, about 175 MDa, about 200 MDa, about 225 MDa, about 250 MDa, about 275 MDa, about 300 MDa, about 325 MDa, about 350 MDa, about 400 MDa, about 425 MDa, about 450 MDa, about 500 MDa, about 550 MDa, about 600 MDa, about 650 MDa, about 700 MDa, about 750 MDa, about 800 MDa, about 850 MDa, about 900 MDa, about 950 MDa, or about 1000 MDa, including all values and ranges therebetween.

In some embodiments, the fusion protein comprises from 1 to about 100 NBPs, from 1 to about 75 NBPs, from 1 to about 50 NBPs, from 1 to about 40 NBPs, from 1 to about 30 NBPs, from 1 to about 20 NBPs, from 1 to about 15 NBPs, from 1 to about 10 NBPs, or from 1 to about 5 NBPs. In embodiments, the fusion protein comprises about 1, about 5, about 10, about 20, about 30, about 40, about 50, about 55, about 60, about 65, about 70, about 75, about 80, about 85, about 90, about 95, or about 100 NBPs. In embodiments, a single polypeptide with phase behavior may be coupled to multiple NBPs, such as about 1 to 100 NBP.

In embodiments, a fusion protein may have two or more NBPs that each bind to different nucleic acids or that bind to the same nucleic acid.

In embodiments, the affinity of a NBP for a nucleic acid is modulated to facilitate separation of the nucleic acid from the fusion protein.

In embodiments, the NBP is selected from any one of RNA-specific adenosine deaminase 1 (ADAR1), ADAR1 double stranded RNA-binding domain 3 (dsRBD3), Bacillus subtilis cold shock protein B (Bs-CspB), cold shock domain Y-box protein (CSD-Ybox), eukaryotic translation initiation factor 4E (eIF4e), Fox-1 protein (FOX1), heterogenous nuclear ribonucleoprotein Q1 (hnRNPQ1), Homo sapiens zinc finger CCCH-Type containing 14 (HsZC3H14), polyA-binding protein (PABP), polyA-binding protein nuclear 1 (PABPN1), pentatricopeptide repeat protein A (PPRpA), Pumilio-Homology Domain (PUM-HD), Pumilio-like repeat protein A (PUFpA), Staufen, 12-O-tetradecanoylphorbol-13-acetate inducible sequence 11 D (TIS11D), Z-DNA/RNA binding protein 1 (ZBP1), and Zinc Finger Nuclease (ZNF). In embodiments, the NBP is ADAR1. In embodiments, the NBP is ADAR1 dsRBD3. In embodiments, the NBP is Bs-CspB. In embodiments, the NBP is CSD-Ybox. In embodiments, the NBP is eIF4e. In embodiments, the NBP is FOX1. In embodiments, the NBP is hnRNPQ1. In embodiments, the NBP is HsZC3H14. In embodiments, the NBP is PABP. In embodiments, the NBP is PABPN1. In embodiments, the NBP is PPRpA. In embodiments, the NBP is PUFpA. In embodiments, the NBP is PUM-HD. In embodiments, the NBP is Staufen. In embodiments, the NBP is TIS11D. In embodiments, the NBP is ZBP. In embodiments, the NBP is ZNF.

In embodiments, the NBP is RNA-specific adenosine deaminase 1 (ADAR1) or a fragment, subunit, or domain thereof. ADAR1 is a polypeptide that catalyzes the post-transcriptional deamination of adenosines, thereby converting them to inosines. ADAR1 binds to and catalyzes double stranded RNA. ADAR1 is described in the following reference, which is incorporated by reference herein in its entirety: Song B, et al. The role of RNA editing enzyme ADAR1 in human disease. Wiley Interdiscip Rev RNA. 2022 January; 13(1):e1665. ADAR1 comprises one or more Z-DNA binding domains, one or more dsRNA binding domains, and a deaminase domain. In embodiments, the NBP is ADAR1 double stranded RNA-binding domain 3 (dsRBD3).

In embodiments, the ADAR1 dsRBD3 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 89.

In embodiments, an NBP comprises a cold shock domain (CSD). CSD contain five antiparallel β-strands that form a β-barrel structure known as an oligosaccharide-/oligonucleotide binding fold. CSD bind to single stranded RNA and single stranded DNA. In embodiments, CSD are comprised of from 60 to 80 amino acids, for example, about 60, about 61, about 62, about 63, about 64, about 65, about 66, about 67, about 68, about 69, about 70, about 71, about 72, about 73, about 74, about 75, about 76, about 77, about 78, about 79, or about 80 amino acids. In embodiments, CSD contain about 70 amino acids. In embodiments, the CSD is a bacterial CSD. In embodiments, bacterial CSD prefer ssDNA to ssRNA by up to ten-fold.

In embodiments, the NBP is Bacillus subtilis cold shock protein B (Bs-CspB) or a fragment, subunit, or domain thereof. Bs-CspB is also known as CspB from Bacillus subtilis (Bscscp). Bs-CspB binds to single stranded DNA (ssDNA) and single stranded RNA (ssRNA). Bs-CspB is a polypeptide that functions as an RNA chaperone and transcriptional anti-terminator. Bs-CspB is described in the following reference, which is incorporated by reference in its entirety herein: Sachs R, et al. RNA single strands bind to a conserved surface of the major cold shock protein in crystals and solution. RNA. 2012 January; 18(1):65-76. Bs-CspB comprises one or more nucleic acid binding motifs.

In embodiments the fusion protein comprises Bs-CspB. In embodiments, Bs-CspB binds to RNA, DNA, or both. In embodiments, Bs-CspB binds to RNA selected from any one of double stranded RNA (dsRNA), single stranded RNA (ssRNA), mRNA, pre-mRNA, polyadenosine (polyA) RNA, Z-confirmation RNA (Z-RNA), or a combination thereof. In embodiments Bs-CspB binds to ssRNA. In embodiments, Bs-CspB binds to the 3′-terminus of mRNA, the 3′-terminus of mRNA, a 3′ untranslated region (UTR) of mRNA, a polyA tail of mRNA, or an AU-rich element, or combination thereof, of mRNA. In embodiments, Bs-CspB binds to binds to an intron, exon, polyA tail, or combination thereof, of the pre-mRNA. In embodiments, Bs-CspB binds to DNA. In embodiments, Bs-CspB binds to single stranded DNA, double stranded DNA, polyadenosine (polyA) DNA, Z-conformation DNA (Z-DNA), or a combination thereof.

In embodiments, the Bs-CspB has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 90.

In embodiments, the NBP is cold shock domain Y-box protein (CSD-Ybox) or a fragment, subunit, or domain thereof. CSD-Ybox is also known as Y-box protein 1 (YB1) cold shock domain (CSD) (YB1-CSD). CSD-Ybox binds to single stranded DNA (ssDNA) and single stranded RNA (ssRNA). CSD-Ybox is a polypeptide that regulates nucleic acid metabolism, e.g., through DNA reparation, pre-mRNA transcription and splicing, mRNA packaging, and regulation of mRNA stability and translation. CSD-Ybox is described in the following reference, which is incorporated by reference in its entirety herein: Heinemann U. and Roske Y. Cold-Shock Domains-Abundance, Structure, Properties, and Nucleic-Acid Binding. Cancers (Basel) 2021 Jan. 7; 13(2):190. CSD-Ybox comprises one or more nucleic acid binding motifs.

In embodiments, the CSD-Ybox has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 91.

In embodiments, the NBP is eukaryotic translation initiation factor 4E (eIF4e) or a fragment, subunit, or domain thereof. eIF4e binds to the 5′-cap of mRNA. eIF4e is a polypeptide that, in synergy with other proteins, binds to mRNA and allows the recruitment of ribosomes for translation initiation. eIF4e is described in the following reference, which is incorporated by reference in its entirety herein: Davis, M. R., et al. Nuclear eIF4E Stimulates 3′-end Cleavage of Target RNAs. Cell Reports, 27, 1397-1408. eIF4e comprises a cap binding pocket.

In embodiments, the eIF4e has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 86, 87, 88, and 96.

In embodiments, the eIF4e has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 86 and 87.

In embodiments, the NBP is Fox-1 protein (FOX1) or a fragment, subunit, or domain thereof. FOX1 binds to pre-mRNA introns and exons. FOX1 is a polypeptide that promotes or represses exon expression by regulating alternative splicing. FOX1 is described in the following reference, which is incorporated by reference in its entirety herein: Kuroyanagi H. Fox-1 family of RNA-binding proteins. Cell Mol Life Sci. 2009 December; 66(24):3895-907. FOX1 comprises an RNA recognition motif (RRM).

In embodiments, the FOX1 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 92.

In embodiments, the NBP is heterogenous nuclear ribonucleoprotein Q1 (hnRNPQ1) or a fragment, subunit, or domain thereof. hnRNPQ1 binds to mRNA. hnRNPQ1 is a polypeptide that regulates mRNA processing events, e.g., pre-mRNA splicing, mRNA transport, and translational regulation. hnRNPQ1 is described in the following reference, which is incorporated by reference in its entirety herein: Xing, L. et al. Negative regulation of RhoA translation and signaling by hnRNP-Q1 affects cellular morphogenesis. Molecular Biology of the Cell 2012 23:8, 1500-1509. hnRNPQ1 comprises one or more RNA recognition motifs (RRM), an acidic domain, and an Arg-Gly-Gly (RGG) box domain.

In embodiments, the hnRNPQ1 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 93.

In embodiments, the NBP is Homo sapiens zinc finger CCCH-Type containing 14 (HsZC3H14) or a fragment, subunit, or domain thereof. HsZC3H14 binds to polyA RNA. HsZC3H14 is a polypeptide that regulates the length of 3′-polyadenosine (polyA) tails. HsZC3H14 is described in the following reference, which is incorporated by reference in its entirety herein: Rha J, et al. The RNA-binding protein, ZC3H14, is required for proper poly(A) tail length control, expression of synaptic proteins, and brain function in mice. Hum Mol Genet. 2017 Oct. 1; 26(19):3663-3681. HsZC3H14 comprises an N-terminal Proline-Tryptophan-Isoleucine (PWI)-like domain and a C-terminal tandem CysCysCysHis (CCCH) Zinc Finger (ZF) domain.

In embodiments, the HsZC3H14 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 94 and 95.

In embodiments, the NBP is polyA-binding protein (PABP) or a fragment, subunit, or domain thereof. PABP binds to polyA RNA. PABP is a polypeptide that mediates mRNA circularization. PABP is described in the following reference, which is incorporated by reference in its entirety herein: Mangus D. A., et al. Poly(A)-binding proteins: multifunctional scaffolds for the post-transcriptional control of gene expression. Genome Biol 4, 223 (2003). PABP comprises one or more RNA recognition motifs (RRM).

In embodiments, the PABP has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 74-85.

In embodiments, the PABP has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 77.

In embodiments, the NBP is polyA-binding protein nuclear 1 (PABPN1) or a fragment, subunit, or domain thereof. PABPN1 binds to polyA RNA tails. PABPN1 is a polypeptide that regulates RNA processing, e.g., prevents the nuclear export of unspliced RNA and regulating the length of polyA tail length. PABPN1 is described in the following reference, which is incorporated by reference in its entirety herein: Mangus D. A., et al. Poly(A)-binding proteins: multifunctional scaffolds for the post-transcriptional control of gene expression. Genome Biol 4, 223 (2003). PABPN1 an RNA recognition motifs (RRM).

In embodiments, the PABPN1 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 97.

In embodiments, the NBP is pentatricopeptide repeat (PPR) or a fragment, subunit, or domain thereof. PPR binds to the 3′ or 5′ termini of RNA transcripts. PPR is a polypeptide that regulates RNA stabilization and translation activation. In embodiments, the NBP is PPR protein a (PPRpA). PPR contain from 20-50 amino acids, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 amino acids. In embodiments, a PPR comprises about 35 amino acids. In embodiments, an NBP comprises a PPR that repeats from about 2-30 times within the NBP sequence. In embodiments, an NBP comprises a PPR that repeats from about 10-30 times within the NBP sequence. In embodiments, the PPR may repeat about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 times within the NBP. In embodiments, the PPR repeats at least 10 times. PPR repeats may be consecutive or separated by one or more amino acids. PPR repeats form two antiparallel α-helices. In embodiments, NBPs comprising a PPR form a solenoid structure. In embodiments, NBPs comprising a PPR bind to single stranded RNA, single stranded DNA., or mRNA. In embodiments, NBPs comprising a PPR bind to the 5′ cap of mRNA. In embodiments, NBPs comprising a PPR bind to the 3′ poly A tail of mRNA. PPR is described in the following reference, which is incorporated by reference in its entirety herein: Manna, S. An overview of pentatricopeptide repeat proteins and their applications, Biochimie, 113, 2015, 93-99. PPR comprises one or more PPR motifs, one or more helix-turn-helix motifs,

In embodiments, the PPRpA has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 98.

In embodiments, the NBP is Pumilio-like repeat (PUF) or a fragment, subunit, or domain thereof. PUF binds to the 3′-UTRs of mRNA in the cytosol and precursors of rRNA in the nucleolus. PUF is a polypeptide that functions as a post-transcriptional and translational regulator. In embodiments, the NBP is a PUF protein A (PUFpA). PUF domains contain eight α-helical repeats of a conserved 36 amino acid sequence that forms a concave RNA binding surface. In embodiments, an NBP comprises from 1-8 of the α-helical repeats of a PUF domain, for example, 1, 2, 3, 4, 5, 6, 7, or 8 α-helical repeats. In embodiments, an NBP comprising a PUF binds to a poly A tail. In embodiments, an NBP comprising a PUF binds to mRNA. PUF is described in the following reference, which is incorporated by reference in its entirety herein: Wang M, et al. The PUF Protein Family: Overview on PUF RNA Targets, Biological Functions, and Post Transcriptional Regulation. Int J Mol Sci. 2018 Jan. 30; 19(2):410. PUF comprises one or more Pumilio-Homology Domain (PUM-HD).

In embodiments, the PUFpA has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 99.

In embodiments, the NBP is Pumilio-Homology Domain (PUM-HD) or a fragment, subunit, or domain thereof. PUM-HD binds to the 3′-UTRs of mRNA and represses their translation. PUM-HD is a polypeptide that functions as a post-transcriptional and translational regulator. PUF is described in the following reference, which is incorporated by reference in its entirety herein: Wang M, et al. The PUF Protein Family: Overview on PUF RNA Targets, Biological Functions, and Post Transcriptional Regulation. Int J Mol Sci. 2018 Jan. 30; 19(2):410. PUM-HD comprises a RNA recognition motif.

In embodiments, the PUM-HD has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 102 and 103.

In embodiments, the NBP is Staufen protein or a fragment, subunit, or domain thereof. Staufen binds to double stranded RNA (dsRNA). Staufen is a polypeptide that functions in RNA trafficking, decay, and translation repression. Staufen is described in the following reference, which is incorporated by reference in its entirety herein: Visentin S, et al. A multipronged approach to understanding the form and function of hStaufen protein. RNA. 2020 March; 26(3):265-277. Staufen comprises one or more dsRNA binding domains (RBDs) and one or more tubulin-binding domains (TBDs).

In embodiments, the Staufen has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 100.

In embodiments, the NBP is 12-O-tetradecanoylphorbol-13-acetate (TPA) inducible sequence 11 D (TIS11D) or a fragment, subunit, or domain thereof. TIS11D binds to AU-rich elements in mRNA. TIS11D is a polypeptide that functions in RNA metabolism and definitive hematopoiesis. TIS11D is described in the following reference, which is incorporated by reference in its entirety herein: Morgan, B. R. et al. Probing the Structural and Dynamical Effects of the Charged Residues of the TZF Domain of TIS11d, 2015 Biophysical Journal, 108:6, 1503-1515. TIS11D comprises one or more CCCH-type tandem zinc finger domains and one or more (R/K)YKTEL motifs.

In embodiments, the TIS11D has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 61-66.

In embodiments, the TIS11D has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 65.

In embodiments, the NBP is Z-DNA/RNA binding protein 1 (ZBP1) or a fragment, subunit, or domain thereof. ZBP1 binds to Z-conformation DNA and RNA. ZBP1 is a polypeptide that functions in the innate immune response by binding to foreign nucleic acids and inducing type-I interferon production. ZBP1 is described in the following reference, which is incorporated by reference in its entirety herein: Maelfait J, et al. Sensing of viral and endogenous RNA by ZBP1/DAI induces necroptosis. EMBO J. 2017 Sep. 1; 36(17):2529-2543. ZBP1 comprises one or more Z-binding domains (ZBDs).

In embodiments, the ZBP1 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NOS: 67-73.

In embodiments, the ZBP1 has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NO: 65.

In embodiments, the NBP is Zinc Finger Nuclease (ZNF) or a fragment, subunit, or domain thereof. ZNF binds to double-stranded DNA and RNA. ZNF is a polypeptide that regulates gene expression at the transcriptional and translational level. ZNF is described in the following reference, which is incorporated by reference in its entirety herein: Chaves-Arquero B, et al. The distinct RNA-interaction modes of a small ZnF domain underlay TUT4(7) diverse action in miRNA regulation. RNA Biol. 2021 Nov. 12; 18(sup2):770-781. ZNF comprises one or more C2H2 domains, one or more CCHC domains, and a catalytic domain.

In embodiments, the ZNF has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of any one of SEQ ID NO: 101.

Described herein is a NBP comprising Bs-CspB. In embodiments, an NBP binds to double stranded RNA. In embodiments, the NBP comprises a dsRNA binding protein (dsRBD) or a fragment thereof. dsRBDs are described in the following reference which is incorporated by reference herein in its entirety: Banerjee et al. RNA Biol. 2014 October; 11(10): 1226-1232.

In embodiments, an NBP binds to capped mRNA. In embodiments, the NBP binding to capped mRNA comprises eukaryotic translation initiation factor 4E (eIF4E), eukaryotic translation initiation factor3 Subunit D (eIF3D), or a combination thereof.

In embodiments, the NBP binds to a groove of DNA or RNA. Non-limiting examples of nucleic acid binding proteins that bind to the groove of DNA or RNA include the trans-activator of transcription (Tat) protein of human immunodeficiency virus-1 (HIV-1), the REV protein of HIV-1, and the RSG-1.2 peptide. RSG-1.2 peptide is a synthetic peptide which binds to the Rev responsive element present within the env gene of the HIV-1 genome. The RSG-1.2 peptide is described in the following article, which is incorporated by reference herein in its entirety: Kumar et al. PLoS One. 2011; 6(8):e23300.

In embodiments, the NBP binds to mRNA. In embodiments, the NBP that binds to mRNA is a ribosomal protein. In embodiments, the ribosomal protein is a 70S ribosome or a 80S ribosome. In embodiments, the ribosomal protein is from the 40S or 60S subunit of the 80S ribosome. In embodiments, the ribosomal protein is from the 30S or 50S subunit of the 70S ribosome. In embodiments, the ribosomal protein is selected from the group consisting of the L3 ribosomal protein, the L4 ribosomal protein, the L13 ribosomal protein, the L20 ribosomal protein, the L22 ribosomal protein, the L24 ribosomal protein, the L24e ribosomal protein, the S12 ribosomal protein, the S14 ribosomal protein, and the eukaryotic initiation factor 4E-binding protein 1 (4EBP1).

In embodiments, the NBP that binds to mRNA is part of the spliceosome. In embodiments, the NBP that is part of the spliceosome is a splicing factor. In embodiments, the splicing factor is selected from the ASF/SF2 splicing factor, serine/arginine rich splicing factor 4 (SRp75), and the serine and arginine rich splicing factor 1 (SRSF1).

In embodiments, the NBP that binds to mRNA is a protein that localizes to p-granules. In embodiments, the protein that localizes to a p-granule is selected from the group consisting of LAF-1, MEG-1, and MEG-3. LAF-1, MEG-1, and MEG-3 are described in the following references, which are incorporated by reference herein in their entirety: Leacock et al. Genetics, Volume 178, Issue 1, 1 Jan. 2008, Pages 295-306; Wu et al. Mol Biol Cell. 2019 Feb. 1; 30(3): 333-345; Elbaum-Garfinkle et al. Proc Natl Acad Sci USA. 2015 Jun. 9; 112(23):7189-94.

In embodiments, the NBP that binds to mRNA is a protein that removes or facilitates removal of the 5′ cap of mRNA, referred to herein as a “decapping protein.” In embodiments, the protein that removes or facilitates removal of the 5′ cap of mRNA is Dcp1, Dcp2, or a combination thereof. Dcp1 and Dcp2 are described in the following reference which is incorporated by reference herein in its entirety: Valkov et al. Nature Structural & Molecular Biology volume 23, pages 574-579 (2016).

In embodiments, the NBP that binds to mRNA is a component of a processing body (p-body). In embodiments, the component of a p-body is Edc3, DHX9, or Xrn1. Components of p-bodies are described in the following reference which is incorporated by reference herein in its entirety: Luo et al. Biochemistry 2018, 57, 17, 2424-2431.

In embodiments, the NBP that binds to mRNA is stem-loop binding protein (SLBP). SLBP binds to the histone 3′ untranslated region (UTR) stem loop structure in replication-dependent histone mRNAs. In embodiments, the NBP that binds to mRNA is a heterogenous nuclear ribonucleoprotein (hnRNP). hnRNPs are described in the following reference which is incorporated by reference herein in its entirety: Geuens et al. Hum Genet. 2016; 135: 851-867.

In embodiments, the NBP that binds to mRNA is GroEL.

In embodiments, the NBP is a protein involved in in vitro transcription. Non-limiting examples of NBPs involved in in vitro transcription include T7 RNA polymerase, Rnase inhibitor, 2′-O-Methyltransferase, Inorganic Pyrophosphatase, Poly(A) Polymerase, DNase I, Calf intestinal phosphatase, Antarctic phosphatase, D1 subunit of the Vaccinia virus mRNA capping enzyme, Guanine-7-methyltransferase (found in D1 subunit of the Vaccinia virus mRNA capping enzyme), Guanylyltransferase (found in D1 subunit of the Vaccinia virus mRNA capping enzyme), RNA triphosphatase (found in D1 subunit of the Vaccinia virus mRNA capping enzyme), and D12 subunit of vaccinia virus mRNA capping enzyme. The following references describe the aforementioned proteins and are incorporated by reference herein in their entirety: Dickson et al. Prog Nucleic Acid Res Mol Biol. 2005; 80: 349-374; Shuman et al. J Biol Chem. 1980 Dec. 10; 255 (23):11588-11598; Luo et al. J Virol. 1995 June; 69(6): 3852-3856; Kobori et al. PNAS Nov. 1, 1984 81 (21) 6691-6695.

In embodiments, the NBP is selected from the group consisting of poly(A)-binding protein (PABP), eukaryotic translation initiation factor 4E (eIF4E), eukaryotic translation initiation factor3 Subunit D (eIF3D), heterogenous nuclear ribonucleoproteins (hnRNPs), RNA-specific adenosine deaminase 1 (ADAR1), RNA-specific adenosine deaminase 2 (ADAR2), CspB from Bacillus subtilis (Bscscp), Y-box protein 1 cold shock domain (YB1-CSD), a Fox-1 protein (FOX1), poly(A)-binding protein (PABP), Staufen protein, TIS11d, zinc finger protein (ZNF), Z-DNA binding protein 1 (ZBP1), retinoic acid-inducible gene-I (RIG-I) like protein, toll like receptor 7 (TLR7), toll like receptor 8 (TLR3), toll like receptor 8 (TLR8), retinoic acid-inducible gene I (RIG-I), melanoma differentiation-associated protein 5 (MDA5), interferon induced protein with tetratricopeptide repeats 1 (IFITI), protein kinase R (PKR), 2′-5′-oligoadenylate synthetase, an oligoadenylate synthase-like (OASL) protein (e.g., OAS1, OAS2, OAS3, or OASL), ribonuclease E (RNASE E), gamma-interferon-inducible protein Ifi-16 (IF116), and cyclic GMP-AMP synthase (cGAS). The following references describe select aforementioned proteins and are incorporated by reference herein in their entirety: Kuroyanagi. Cell Mol Life Sci. 2009; 66(24): 3895-3907; Baou et al. J Biomed Biotechnol. 2009; 2009: 634520; and Brisse and Ly. Front. Immunol., 17 Jul. 2019; 10: 1586; Rehwinkel et al. Nature Reviews Immunology volume 20, pages537-551 (2020); and Brisse et al. Front Immunol. 2019; 10: 1586; Luo et al. Cell. 2011 Oct. 14; 147(2): 409-422.

In embodiments, the NBP comprises one or more RNA binding domains (RBDs) and one or more intrinsically disordered regions (IDRs). In embodiments, the IDR comprises an RG[G] repeat, an RS/RG rich domain, a K/R patch, molecular recognition features, a low complexity sequence, a pentatricopeptide domain, or a combination thereof.

In embodiments, the NBP comprises one or more of the following domains: a short linear motif (SLiM), an RG repeat, an RGG repeat, a RS/RG rich domain, a K/R basic patch, a molecular recognition feature, a low complexity sequence, an RNA recognition motif, a double-stranded RNA binding domain, a K homology domain, a zinc finger domain (e.g., CCHH ZF domain, a CCCC (Ran-BP2) domain, a CCCH ZF domain), an RGG domain, a Pumillo family domain, a pentatricopeptide domain, a cold shock domain, a helicase domain, a La motif, a Piwi-Argonaute-Zwille (PAZ) domain, a P-element induced wimpy testis, a pseudouridine synthase and archaeosine transglycosylate (PUA), a Pumillo-like repeat (PUM), a ribosomal S1-like (S1), Sm and Like-Sm (Sm/Lsm) repeat, thiouridine synthases and RNA methylases and pseudouridine synthases (THUMP), and a domain with YT521-B homology. The following references describe many of these domains and are incorporated by reference herein in their entireties: Balcerak et al. Open Biol. 2019 June; 9(6) 190096; Jarvelin et al. Cell Commun Signal, 2016: 14, 9; Corley et al. Mol. Cell. 2020 Apr. 2; 78(1): 9-29; De Franco et al. Sci Rep: 2019: 9, 2484; Shotwell et al. 2020. Wiley Interdiscip Rev RNA, 11, e1573; Simon et al. 2019. Molecular Cell, 75, 66-75.e5; Varadi et al, 2015, PLoS One, 10, e0139731; Zeke et al, 2020, WIREs RNA, n/a, e1714.

In embodiments, the NBP comprises a short linear motif (SLiM). A SLiM is composed of up to ten amino acid residue motifs located predominantly outside protein domains. SLiMs bind to RNA with low affinity in a non-specific manner. SLiMs are often repeated multiple times throughout a protein.

In embodiments, a NBP comprises a pseudouridine synthase and archaeosine transglycosylase (PUA) domain. PUA domains range from 67-94 amino acids in length, with a β₁α₁β₂β₃β₄β₃α₂β₆ architecture that forms a pseudobarrel encased by two α-helices. In embodiments, an NBP comprising a PUA binds to double stranded RNA.

In embodiments, a NBP comprises a S1 RNA binding domain. In embodiments, an NBP comprising a S1 RNA binding domain interacts with single stranded RNA, double stranded RNA, or mRNA. In embodiments, the S1 RNA binding domain comprises from about 60 to about 80 amino acids, for example, about 60, about 61, about 62, about 63, about 64, about 65, about 66, about 67, about 68, about 69, about 70, about 71, about 72, about 73, about 74, about 75, about 76, about 77, about 78, about 79, or about 80 amino acids. In embodiments, the S1 RNA binding domain comprises about 70 amino acids.

In embodiments, a NBP comprises an Sm RNA binding motif. Sm RNA binding motifs are found in Sm and Like-sm (Lsm) proteins in eukaryotes and archaea and in Hfq proteins in prokaryotes. The Sm motif consists of ˜70 residues with an α₁β₁β₂β₃β₄β₅ topology that forms a curved antiparallel β-sheet. Sm-containing proteins readily multimerize through interactions between strands 04 and 05 in two Sm motifs. In embodiments, a NBP comprises 1, 2, 3, 4, 5, 6, 7, 8, 9, or 10 Sm motifs. In embodiments, an NBP comprises two Sm motifs. In embodiments, an Sm binding motif binds to RNA through hydrogen bonding and base stacking interactions.

In embodiments, a NBP comprises a thiouridine synthase and RNA methylase and pseudouridine synthase (THUMP) domain. The THUMP domain is found in many tRNA-modifying enzymes. THUMP domains are found in proximity to RNA-modifying domains and sometimes in proximity to an N-terminal ferredoxin-like domain. THUMP domains display a α₁α₂β₁α₃β₂β₂ topology that forms parallel α-helices flanking a β-sheet. In embodiments, an NBP comprising a THUMP domain binds to tRNA.

In embodiments, a NBP comprises YT521-B homology domain. In embodiments, a YT521-B homology domains comprises from 100-150 amino acids, for example, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, or about 150 amino acids. In embodiments, a NBP comprising a YT521-B homology domain binds to a methylated adenosine.

In embodiments, a NBP comprises a Piwi-Argonautre-Zwille (PAZ) domain. In embodiments, a PAZ domain facilitates binding of small interfering mRNA and/or microRNA guides to mRNA targets. In embodiments, the PAZ domain is from a Dicer protein or an Argonaute protein. PAZ domains display a six-stranded β-barrel topped with two α-helices and flanked on the opposite side by a special appendage containing a β-hairpin and short α-helix.

In embodiments, a NBP comprises a P-element induced Wimpy Testis (PIWI) domain. In embodiments, a PIWI domain facilitates binding of small interfering mRNA and/or microRNA guides to mRNA targets. In embodiments, a PIWI domain is found on an Argonaute protein. The PIWI domain tertiary structure forms an RNase H-like fold consisting of a five-stranded β-sheet flanked by α-helices on both faces.

In embodiments, a NBP comprises a PAZ domain and a PIWI domain.

In embodiments, the NBP comprises an RS/RG rich domain. RS/RG rich domains contain repeats of arginine-serine (RS), arginine-glycine (RG), or a combination thereof. RS/RG rich domains mediate specific or non-specific interactions with RNA. Examples of proteins containing RS/RG rich domains include the SR proteins and SR-like proteins like serine/arginine-rich splicing factor 1 (SRSF1) and RNA-helicase DDX23.

In embodiments, an NBP comprises a helicase domain. Helicases comprise six superfamilies (SFs), including SF1, SF2, SF3, SF4, SF5, and SF6. In embodiments, the helicase domain is a eukaryotic RNA and DNA helicase from the SF1 or SF2 superfamilies. Non-limiting examples of families within the SF1 and SF2 superfamilies include the Upf1-like family, the DEAD-box, DEAH, RIG-I-like, Ski2-like, and NS3 families. In embodiments, the helicase domain is a bacterial or viral helicase from the SF3, SF4, SF5, or SF6 superfamily. ATP binding to a helicase promotes higher affinity of a helicase domain to RNA. ATP hydrolysis promotes conformational changes that cause the helicase to unwind its substrate and/or translocate one nucleotide.

In embodiments, a NBP comprises a La motif. The La motif consists of five α-helices and three β-strands that form a small antiparallel β-sheet against a modified “winged-helix” fold. In embodiments, the La motif binds to 3′-terminal UUU-OH elements on polymerase III transcribed small RNAs. La motifs comprise between 80 and 100 amino acids, for example, about 80, about 81, about 82, about 83, about 84, about 85, about 86, about 87, about 88, about 89, about 90, about 91, about 92, about 93, about 94, about 95, about 96, about 97, about 98, about 99, or about 100 amino acids. In embodiments, a La motif comprises about 90 amino acids.

In embodiments, the NBP comprises a RG[G] repeat. RG[G] repeats are known to have broad, degenerate binding. RG[G] repeats are motifs rich in arginine and glycine consisting of at least three RG/RGG repeats (e.g., from 3-500), separated by 10 amino acid residues. RG/RGG motifs include RGG and/or RG repeats of varied lengths interspersed with spacers of different amino acids. In embodiments, an NBP comprises a di-RGG motif. Di-RGG motifs contain two repeated RGG sequences separated by 0-4 amino acids. In embodiments, an NBP comprises a di-RG motif. Di-RG motifs contain two repeated RG sequences separated by 0-4 amino acids. In embodiments, an NBP comprises a tri-RGG motif. Tri-RGG motifs contain three repeated RGG sequences separated by 0-4 amino acids. In embodiments, an NBP comprises a tri-RG motif. Tri-RG motifs contain three repeated RG sequences separated by 0-4 amino acids. These motifs are described in the following article which is incorporated by reference herein in its entirety: Thandapani et al. (2013). Molecular Cell, 50, 613-623.

In embodiments, the amino acid sequence of the NBP comprises one or more RG, RGG, RGGR, RGGGR, or a combination thereof. In embodiments, NBPs comprising RG, RGG, RGGR, or RGGGR or a combination thereof mediate hydrogen bonding and base stacking with DNA and RNA via the arginine moieties. In embodiments, NBPs comprising RG, RGG, RGGR, RGGGR or a combination thereof bind to DNA G-quadruplexes. An exemplary protein containing a repeat of RGG, RGGR, or RGGGR is the RNA binding protein FUS. In embodiments, the NBP comprises FUS. In embodiments, the NBP sequence contains consecutive repeats of RGG, RGGR, RGGGR, or combinations thereof. An exemplary NBP containing a combination of RGG, RGGR, or RGGGR repeats may comprise the sequence RGGRGGRGGRRGGRRGGRRGGGRRGG. In embodiments, an NBP may comprise one or more RGG, RGGR, or RGGGR interspersed throughout its sequence. In embodiments, an NBP contains from 1 to 100 RG, RGG, RGGR, or RGGGR sequences. The RGG, RGGR, and RGGGR may be interspersed throughout the sequence (separated by one or more amino acids) or consecutive. In embodiments, the NBP comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 RG, RGG, RGGR, or RGGGR repeats. The RG, RGG, RGGR, and RGGGR repeats may be consecutive or interspersed throughout the sequence. The following article describes exemplary RGG sequences and is incorporated by reference herein in its entirety: Simon et al. Molecular Cell (2019), 75, 66-75.e5.

In embodiments, an NBP comprises an RG domain. An RG domain comprises from about 2 to about 500 repeats of RG (arginine-glycine). In embodiments, an NBP comprises an RGG domain. An RGG domain comprises from about 2 to about 500 repeats of RGG (arginine-glycine-glycine). In embodiments, an NBP comprises an RGGR domain. An RGGR domain comprises from about 2 to about 500 repeats of RGGR (arginine-glycine-glycine-arginine). In embodiments, an NBP comprises an RGGGR domain. An RGGGR domain comprises from about 2 to about 500 repeats of RGG (arginine-glycine-glycine-glycine-arginine). In embodiments, an NBP comprise an RG mix domain. An RG mix domain comprises 2-500 simultaneous repeats of RG, RGG, RGGR, and/or RGGGR. For example, the RG mix domain may comprise RGG, followed by RG, followed by RGGR, followed by RG, followed by RGGGR.

In embodiments, the NBP comprises a K/R basic patch. A K/R basic patch contains from 4-8 consecutive lysines, arginines, or a combination thereof. K/R basic patches form a highly positive and exposed interface which binds to RNA. K/R basic patches are frequently contained in multiple clusters on the same protein.

In embodiments, the NBP comprises a molecular recognition feature (MoRF). In embodiments, the MoRF is up to 25 amino acids long, 50 or more amino acids long, or from 25 to 50 amino acids in length. MoRFs undergo a dynamic disorder-to-order transition upon ligand binding.

In embodiments, the NBP comprises a low complexity (LC) sequence. In embodiments, LC sequences contain up to 100 amino acids and are composed of many repeats of the same amino acid or several amino acid. LC sequences can polymerize into amyloid-like fibers and undergo reversible phase transition to a hydrogel-like state. Examples of proteins containing LC sequences are FUS and hnRNPA2.

In embodiments, the NBP comprises a RNA recognition motif (RRM). RRMs bind to RNA. Typically, binding is sequence-specific. In embodiments, RRMs comprise from about 75 to about 125 amino acids, for example, about 75, about 80, about 85, about 90, about 95, about 100, about 105, about 110, about 115, about 120, or about 125 amino acids in length. In embodiments, an RRM comprises about 85 amino acids. RRMs typically adopt a β₁α₁β₂β₃α₂β₄ topology forming two alpha-helices against an antiparallel beta sheet, which houses the conserved RNA-binding RNP1 and RNP2 motifs in central β₁ and β₃ strands.

In embodiments, the NBP comprises a double stranded RNA-binding domain (dsRBD). In embodiments, a dsRBD comprises from about 55 to about 80 amino acids, or from about 65 to about 70 amino acids. In embodiments, a dsRBD comprises 68 amino acids. dsRBD typically adopt an αβββα conformation. In embodiments, a dsRBD occurs as a tandem repeats or in combination with other RNA binding domains. There are two subclasses of dsRBDs, type B and type A. Type A has better binding to dsRNA than type B. dsRBDs typically bind in a shape dependent fashion and not sequence specific. However, ADAR2 is a rare example of a dsRBD that exhibits sequence specific binding.

In embodiments, the NBP comprises a K homology domain. In embodiments, the K homology domain comprises from 60 to 80 amino acids. In embodiments, the K homology domain comprises 70 amino acids. There are two types of K homology domains: type I or reverse type II. The type I K homology domain adopts the β₁α₁α₂β₂β′α′ topology. The reverse type II K homology domain adopts the α′β′β₁α₁α₂β₂ topology. K homology domains do not use aromatic amino acids for binding and instead use hydrogen bonding. NBP containing K homology domain are difficult to design due to their stringent sequence specificity.

In embodiments, the NBP comprises one or more zinc finger (ZF) domains. In embodiments, the NBP comprises from 1-100 ZF domains, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 ZF domains. In embodiments, the zinc finger domain is selected from one of the following subtypes: CCHC (zinc knuckle), CCCH, CCCC (RanBP2), and CCHH. C and H refer to the interspersed cysteine and histidine residues that coordinate the zinc atom. In embodiments, a zinc finger domain comprises from about 20 to about 40 amino acids, for example, about 20, about 22, about 24, about 26, about 28, about 30, about 32, about 34, about 36, about 38, or about 40 amino acids. CCHH ZF domains contain two conserved cysteine and two conserved histidine residues. CCHH ZF domains recognize both structural and sequence specific elements. To this date, there are no engineered versions of CCHH ZF domains. CCHH ZF domains bind both single stranded and double stranded DNA and RNA. CCCC ZFs might not require a specific RNA conformation for binding. Typically, CCCC ZFs recognize short three nucleotide repeats. An engineered version of the CCCC ZF is described in the following reference which is incorporated by reference herein in its entirety: De Franco et al. Sci Rep: 2019: 9, 2484.

In embodiments, the nucleic acid binding protein (NBP) is a polypeptide from Table 1 or a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of the polypeptides of Table 1. In embodiments, the NBP has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 90.

TABLE 1
Exemplary amino acid sequences of NBP.

| SEQ ID NO | SEQUENCE | IDENTITY |
|---|---|---|
| 61 | MSTTLLSAFYDVDFLCKTEKSLANLNLNNMLDKKAVGTPVAAAPSSGFAPG | TIS11D |
| | FLRRHSASNLHALAHPAPSPGSCSPKFPGAANGSSCGSAAAGGPTSYGTLKE | |
| | PSGGGGTALLNKENKFRDRSFSENGDRSQHLLHLQQQQKGGGGSQINSTRY | |
| | KTELCRPFEESGTCKYGEKCQFAHGFHELRSLTRHPKYKTELCRTFHTIGFCP | |
| | YGPRCHFIHNADERRPAPSGGASGDLRAFGTRDALHLGFPREPRPKLHHSLS | |
| | FSGFPSGHHQPPGGLESPLLLDSPTSRTPPPPSCSSASSCSSSASSCSSASAAST | |
| | PSGAPTCCASAAAAAAAALLYGTGGAEDLLAPGAPCAACSSASCANNAFAF | |
| | GPELSSLITPLAIQTHNFAAVAAAAYYRSQQQQQQQGLAPPAQPPAPPSATL | |
| | PAGAAAPPSPPFSFQLPRRLSDSPVFDAPPSPPDSLSDRDSYLSGSLSSGSLSG | |
| | SESPSLDPGRRLPIFSRLSISDD | |
| 62 | STTLLSAFYDVDFLCKTEKSLANLNLNNMLDKKAVGTPVAAAPSSGFAPGF | TIS11D |
| | LRRHSASNLHALAHPAPSPGSCSPKFPGAANGSSCGSAAAGGPTSYGTLKEP | |
| | SGGGGTALLNKENKFRDRSFSENGDRSQHLLHLQQQQKGGGGSQINSTRYK | |
| | TELCRPFEESGTCKYGEKCQFAHGFHELRSLTRHPKYKTELCRTFHTIGFCPY | |
| | GPRCHFIHNADERRPAPSGGASGDLRAFGTRDALHLGFPREPRPKLHHSLSF | |
| | SGFPSGHHQPPGGLESPLLLDSPTSRTPPPPSCSSASSCSSSASSCSSASAASTP | |
| | SGAPTCCASAAAAAAAALLYGTGGAEDLLAPGAPCAACSSASCANNAFAF | |
| | GPELSSLITPLAIQTHNFAAVAAAAYYRSQQQQQQQGLAPPAQPPAPPSATL | |
| | PAGAAAPPSPPFSFQLPRRLSDSPVFDAPPSPPDSLSDRDSYLSGSLSSGSLSG | |
| | SESPSLDPGRRLPIFSRLSISDD | |
| 63 | MSTRYKTELCRPFEESGTCKYGEKCQFAHGFHELRSLTRHPKYKTELCRTFH | TIS11D |
| | TIGFCPYGPRCHFIHNADE | |
| 64 | MYGEKCQFAHGFHELRSLTRHPKYKTELCRTFHTIGFCPYGPR | TIS11D |
| 65 | STRYKTELCRPFEESGTCKYGEKCQFAHGFHELRSLTRHPKYKTELCRTFHTI | TIS11D |
| | GFCPYGPRCHFIHNADE | |
| 66 | YGEKCQFAHGFHELRSLTRHPKYKTELCRTFHTIGFCPYGPR | TIS11D |
| 67 | MAQAPADPGREGHLEQRILQVLTEAGSPVKLAQLVKECQAPKRELNQVLY | ZBP |
| | RMKKELKVSLTSPATWCLGGTDPEGEGPAELALSSPAERPQQHAATIPETPG | |
| | PQFSQQREEDIYRFLKDNGPQRALVIAQALGMRTAKDVNRDLYRMKSRHLL | |
| | DMDEQSKAWTIYRPEDSGRRAKSASIIYQHNPINMICQNGPNSWISIANSEAI | |
| | QIGHGNIITRQTVSREDGSAGPRHLPSMAPGDSSTWGTLVDPWGPQDIHME | |
| | QSILRRVQLGHSNEMRLHGVPSEGPAHIPPGSPPVSATAAGPEASFEARIPSP | |
| | GTHPEGEAAQRIHMKSCFLEDATIGNSNKMSISPGVAGPGGVAGSGEGEPGE | |
| | DAGRRPADTQSRSHFPRDIGQPITPSHSKLTPKLETMTLGNRSHKAAEGSHY | |
| | VDEASHEGSWWGGGI | |
| 68 | AQAPADPGREGHLEQRILQVLTEAGSPVKLAQLVKECQAPKRELNQVLYR | ZBP1 |
| | MKKELKVSLTSPATWCLGGTDPEGEGPAELALSSPAERPQQHAATIPETPGP | |
| | QFSQQREEDIYRFLKDNGPQRALVIAQALGMRTAKDVNRDLYRMKSRHLL | |
| | DMDEQSKAWTIYRPEDSGRRAKSASIIYQHNPINMICQNGPNSWISIANSEAI | |
| | QIGHGNIITRQTVSREDGSAGPRHLPSMAPGDSSTWGTLVDPWGPQDIHME | |
| | QSILRRVQLGHSNEMRLHGVPSEGPAHIPPGSPPVSATAAGPEASFEARIPSP | |
| | GTHPEGEAAQRIHMKSCFLEDATIGNSNKMSISPGVAGPGGVAGSGEGEPGE | |
| | DAGRRPADTQSRSHFPRDIGQPITPSHSKLTPKLETMTLGNRSHKAAEGSHY | |
| | VDEASHEGSWWGGGI | |
| 69 | MPGREGHLEQRILQVLTEAGSPVKLAQLVKECQAPKRELNQVLYRMKKEL | ZBP |
| | KVSLTSPATWCLGG | |
| 70 | PGREGHLEQRILQVLTEAGSPVKLAQLVKECQAPKRELNQVLYRMKKELKV | ZBP1 |
| | SLTSPATWCLGG | |
| 71 | MPQFSQQREEDIYRFLKDNGPQRALVIAQALGMRTAKDVNRDLYRMKSRH | ZBP1 |
| | LLDMDEQSKAWTIYR | |
| 72 | PQFSQQREEDIYRFLKDNGPQRALVIAQALGMRTAKDVNRDLYRMKSRHLL | ZBP1 |
| | DMDEQSKAWTIYR | |
| 73 | SPQFSQQREEDIYRFLKDNGPQRALVIAQALGMRTAKDVNRDLYRMKSRHL | ZBP1 |
| | LDMDEQSKAWTIYRWTIY | |
| 74 | MNPSAPSYPMASLYVGDLHPDVTEAMLYEKFSPAGPILSIRVCRDMITRRSL | PABP |
| | GYAYVNFQQPADAERALDTMNFDVIKGKPVRIMWSQRDPSLRKSGVGNIFI | |
| | KNLDKSIDNKALYDTFSAFGNILSCKVVCDENGSKGYGFVHFETQEAAERAI | |
| | EKMNGMLLNDRKVFVGRFKSRKEREAELGARAKEFTNVYIKNFGEDMDDE | |
| | RLKDLFGKFGPALSVKVMTDESGKSKGFGFVSFERHEDAQKAVDEMNGKE | |
| | LNGKQIYVGRAQKKVERQTELKRKFEQMKQDRITRYQGVNLYVKNLDDGI | |
| | DDERLRKEFSPFGTITSAKVMMEGGRSKGFGFVCFSSPEEATKAVTEMNGRI | |
| | VATKPLYVALAQRKEERQAHLTNQYMQRMASVRAVPNPVINPYQPAPPSG | |
| | YFMAAIPQTQNRAAYYPPSQIAQLRPSPRWTAQGARPHPFQNMPGAIRPAAP | |
| | RPPFSTMRPASSQVPRVMSTQRVANTSTQTMGPRPAAAAAAATPAVRTVPQ | |
| | YKYAAGVRNPQQHLNAQPQVTMQQPAVHVQGQEPLTASMLASAPPQEQK | |
| | QMLGERLFPLIQAMHPTLAGKITGMLLEIDNSELLHMLESPESLRSKVDEAV | |
| | AVLQAHQAKEAAQKAVNSATGVPTV | |
| 75 | NPSAPSYPMASLYVGDLHPDVTEAMLYEKFSPAGPILSIRVCRDMITRRSLG | PABP |
| | YAYVNFQQPADAERALDTMNFDVIKGKPVRIMWSQRDPSLRKSGVGNIFIK | |
| | NLDKSIDNKALYDTFSAFGNILSCKVVCDENGSKGYGFVHFETQEAAERAIE | |
| | KMNGMLLNDRKVFVGRFKSRKEREAELGARAKEFTNVYIKNFGEDMDDER | |
| | LKDLFGKFGPALSVKVMTDESGKSKGFGFVSFERHEDAQKAVDEMNGKEL | |
| | NGKQIYVGRAQKKVERQTELKRKFEQMKQDRITRYQGVNLYVKNLDDGID | |
| | DERLRKEFSPFGTITSAKVMMEGGRSKGFGFVCFSSPEEATKAVTEMNGRIV | |
| | ATKPLYVALAQRKEERQAHLTNQYMQRMASVRAVPNPVINPYQPAPPSGY | |
| | FMAAIPQTONRAAYYPPSQIAQLRPSPRWTAQGARPHPFQNMPGAIRPAAPR | |
| | PPFSTMRPASSQVPRVMSTQRVANTSTQTMGPRPAAAAAAATPAVRTVPQY | |
| | KYAAGVRNPQQHLNAQPQVTMQQPAVHVQGQEPLTASMLASAPPQEQKQ | |
| | MLGERLFPLIQAMHPTLAGKITGMLLEIDNSELLHMLESPESLRSKVDEAVA | |
| | VLQAHQAKEAAQKAVNSATGVPTV | |
| 76 | MSYPMASLYVGDLHPDVTEAMLYEKFSPAGPILSIRVCRDMITRRSLGYAY | PABP |
| | VNFQQPADAERALDTMNFDVIKGKPVRIMWSQRDPSLRKSGVGNIFIKNLD | |
| | KSIDNKALYDTFSAFGNILSCKVVCDENGSKGYGFVHFETQEAAERAIEKM | |
| | NGMLLNDRKVFVGRFKSRKER | |
| 77 | SYPMASLYVGDLHPDVTEAMLYEKFSPAGPILSIRVCRDMITRRSLGYAYVN | PABP |
| | FQQPADAERALDTMNFDVIKGKPVRIMWSQRDPSLRKSGVGNIFIKNLDKSI | |
| | DNKALYDTFSAFGNILSCKVVCDENGSKGYGFVHFETQEAAERAIEKMNG | |
| | MLLNDRKVFVGRFKSRKER | |
| 78 | MASLYVGDLHPDVTEAMLYEKFSPAGPILSIRVCRDMITRRSLGYAYVNFQ | PABP |
| | QPADAERALDTMNFDVIKGKPVRIMWSQR | |
| 79 | ASLYVGDLHPDVTEAMLYEKFSPAGPILSIRVCRDMITRRSLGYAYVNFQQP | PABP |
| | ADAERALDTMNFDVIKGKPVRIMWSQR | |
| 80 | MGNIFIKNLDKSIDNKALYDTFSAFGNILSCKVVCDENGSKGYGFVHFETQE | PABP |
| | AAERAIEKMNGMLLNDRKVFVGRFKS | |
| 81 | GNIFIKNLDKSIDNKALYDTFSAFGNILSCKVVCDENGSKGYGFVHFETQEA | PABP |
| | AERAIEKMNGMLLNDRKVFVGRFKS | |
| 82 | MTNVYIKNFGEDMDDERLKDLFGKFGPALSVKVMTDESGKSKGFGFVSFER | PABP |
| | HEDAQKAVDEMNGKELNGKQIYVGRAQK | |
| 83 | TNVYIKNFGEDMDDERLKDLFGKFGPALSVKVMTDESGKSKGFGFVSFERH | PABP |
| | EDAQKAVDEMNGKELNGKQIYVGRAQK | |
| 84 | MVNLYVKNLDDGIDDERLRKEFSPFGTITSAKVMMEGGRSKGFGFVCFSSP | PABP |
| | EEATKAVTEMNGRIVATKPLYVALAQR | |
| 85 | VNLYVKNLDDGIDDERLRKEFSPFGTITSAKVMMEGGRSKGFGFVCFSSPEE | PABP |
| | ATKAVTEMNGRIVATKPLYVALAQR | |
| 86 | KHPLQNRWALWFFKNDKSKTWQANLRLISKFDTVEDFWALYNHIQLSSNL | eIF4e |
| | MPGCDYSLFKDGIEPMWEDEKNKRGGRWLITLNKQQRRSDLDRFWLETLL | |
| | CLIGESFDDYSDDVCGAVVNVRAKGDKIAIWTTECENREAVTHIGRVYKER | |
| | LGLPPKIVIGYQSHADTATKSGSTTKNRFVV | |
| 87 | MKHPLQNRWALWFFKNDKSKTWQANLRLISKFDTVEDFWALYNHIQLSSN | eIF4e |
| | LMPGCDYSLFKDGIEPMWEDEKNKRGGRWLITLNKQQRRSDLDRFWLETL | |
| | LCLIGESFDDYSDDVCGAVVNVRAKGDKIAIWTTECENREAVTHIGRVYKE | |
| | RLGLPPKIVIGYQSHADTATKSGSTTKNRFVV | |
| 88 | MATVEPETTPTPNPPTTEEEKTESNQEVANPEHYIKHPLQNRWALWFFKND | eIF4e |
| | KSKTWQANLRLISKFDTVEDFWALYNHIQLSSNLMPGCDYSLFKDGIEPMW | |
| | EDEKNKRGGRWLITLNKQQRRSDLDRFWLETLLCLIGESFDDYSDDVCGAV | |
| | VNVRAKGDKIAIWTTECENREAVTHIGRVYKERLGLPPKIVIGYQSHADTAT | |
| | KSGSTTKNRFVV | |
| 89 | VRKIGELVRYLNTNPVGGLLEYARSHGFAAEFKLVDQSGPPHEPKFVYQAK | ADAR1 |
| | VGGRWFPAVCAHSKKQGKQEAADAALRVLIGENEKAER | |
| 90 | LEGKVKWFNSEKGFGFIEVEGQDDVFVHFSAIQGEGFKTLEEGQAVSFEIVE | Bs-CspB |
| | GNRGPQAANVTKEA | |
| 91 | GKKVIATKVLGTVKWFNVRNGYGFINRNDTKEDVFVHQTAIKKNNPRKYL | CSD-Ybox |
| | RSVGDGETVEFDVVEGEKGAEAANVTGP | |
| 92 | NTENKSQPKRLHVSNIPFRFRDPDLRQMFGQFGKILDVEIIFNERGSKGFGFV | FOX1 |
| | TFENSADADRAREKLHGTVVEGRKIEVNNATARVMTNKKTVNPYTN | |
| 93 | DSVYSGQQPSVGTEIFVGKIPRDLFEDELVPLFEKAGPIWDLRLMMDPLTGL | hnRNPQ1 |
| | NRGYAFVTFCTKEAAQEAVKLYNNHEIRSGKHIGVCISVANNRLFVGSIPKS | |
| | KTKEQILEEFSKVTEGLTDVILYHQPDDKKKNRGFCFLEYEDHKTAAQARR | |
| | RLMSGKVKVWGNVGTVEWADPIEDPDPEVMAKVKVLFVRNLANTVTEEIL | |
| | EKAFSQFGKLERVKKLKDYAFIHFDERDGAVKAMEEMNGKDLEGENIEIVF | |
| | AKPPD | |
| 94 | MSELSVAQKPEKLLERCKYWPACKNGDECAYHHPISPCKAFPNCKFAEKCL | HsZC3H14 |
| | FVHPNCKYDAKCTKPDCPFTHVSRRIPVLSPKPAVAPPAPPSSSQLCRYFPAC | |
| | KKMECPFYHPKHCRFNTQCTRPDCTFYHPTINVPPRHALKWIRPQTSE | |
| 95 | SELSVAQKPEKLLERCKYWPACKNGDECAYHHPISPCKAFPNCKFAEKCLF | HsZC3H14 |
| | VHPNCKYDAKCTKPDCPFTHVSRRIPVLSPKPAVAPPAPPSSSQLCRYFPACK | |
| | KMECPFYHPKHCRFNTQCTRPDCTFYHPTINVPPRHALKWIRPQTSE | |
| 96 | ATVEPETTPTPNPPTTEEEKTESNQEVANPEHYIKHPLQNRWALWFFKNDKS | eIF4e |
| | KTWQANLRLISKFDTVEDFWALYNHIQLSSNLMPGCDYSLFKDGIEPMWED | |
| | EKNKRGGRWLITLNKQQRRSDLDRFWLETLLCLIGESFDDYSDDVCGAVVN | |
| | VRAKGDKIAIWTTECENREAVTHIGRVYKERLGLPPKIVIGYQSHADTATKS | |
| | GSTTKNRFVV | |
| 97 | GAIEDPELEAIKARVREMEEEAEKLKELQNEVEKQMNMSPPPGNAGPVIMSI | PABPN1 |
| | EEKMEADARSIYVGNVDYGATAEELEAHFHGCGSVNRVTILCDKFSGHPKG | |
| | FAYIEFSDKESVRTSLALDESLFRGRQIKVIPKRTNRPGISTTDRGFPRARYRA | |
| | RTTNYNSSRSRFYSGFNSRPRGRVYRG | |
| 98 | GNSVTYSTLISGLGKAGRLEEALELFEEMKEKGIVPTVVTYTTLINGLGKAG | PPRpA |
| | KLDEALELFEEMKEKGLVPNIVTYTSLINGLGKAGRVEEALELFDEMKERG | |
| | VVPNVVTFSTLISGFARAGRLEEALELFEEMKEKGIVPSVVTYTTLIDGLCKA | |
| | GKLDEALKLFEEMVEKGIKPNVVTYTTLIDGLGRAGKLDEALKLFEEMVEK | |
| | GIKPNVVTYSTLIDGLCKAGKLDEALKLFEEMVEKGIKPTVELTYRRVVESY | |
| | CRAKRFE | |
| 99 | GRSRLLEDFRNNRYPNLQLREIAGHIMEFSQDQHGSRFIQLKLERATPAERQ | PUFpA |
| | LVFNEILQAAYQLMVDVFGVFVIQKFFEFGSLEQKLALAERIRGHVLSLALQ | |
| | MYGTRVIQKALEFIPSDQQNEMVRELDGHVLKCVKDQNGRGVVYKCIECV | |
| | QPQSLQFIIDAFKGQVFALSTHPYGPHVIQRILEHCLPDQTLPILEELHQHTEQ | |
| | LVQDQYGVWVIHHVLEHGRPEDKSKIVAEIRGNVLVLSQHKFAMHVVQKC | |
| | VTHASRTERAVLIDEVCTMNDGPHSALYTMMKDQYAQLVVRKMIDVAEPG | |
| | QRKIVMHKIRPHIATLRKYTYGKHILAKLEKYYMKNGVDLG | |
| 100 | DKKSPISQVHEIGIKRNMTVHFKVLREEGPAHMKNFITACIVGSIVTEGEGNG | Staufen |
| | KKVSKKRAAEKMLVELQKL | |
| 101 | YVCHFENCGKAFKKHNQLKVHQFSHTQQLPYECPHEGCDKRFSLPSRLKRH | ZNF |
| | EKVHAGYPCKKDDSCSFVGKTWTLYLKHVAECHQD | |
| 102 | MHRGNEDLSFGDDYEKEIGLLLGEQQRRQEEADEIEKELNLYRSGSAPPTVD | PUM-HD |
| | GSVNAAGGLFNGGGRGPFMEFGGGNKGNGFGGDDDELRKDPAYLSYYYA | |
| | NMKLNPRLPPPLMSREDLRVAQRLKGSSNVLGGVGDRRNVNESRSLFSMPP | |
| | GFDQMNEFEAEKTNASSSEWDANGLIGLPGLGLGGKQKSFADIFQPDMGHP | |
| | VSQQPSRPASRNAFDENVDSTNNQSPSASQGIGAPPPYSYAAVLGSSLSRNG | |
| | TPDPQAVARVPSPCLTPIGSGRVSSNDKRNTSNQSPFNGVTSGLNESSDLVN | |
| | ALSGMNLSGSGGLDDRGQAEQDVEKVRNYMFGFQGGHNEVSQHVFPNKS | |
| | DQAQKATGSLRNLHMRGSQGSAYNGGGLANPYQHLDSPNYCLNNYALNP | |
| | AVASVMANQLGNSNFSPMYDNYSAASALGFSGMDSRLHGGGFESRNLGRS | |
| | NRMMGGGGLQSHMADPMYHQYGRYSENVDALDLLNDPAMDRSFMGNSY | |
| | MNMLELQRAYLGAQKSQYGVPYKSGSPNSHSYYGSPTFGSNMSYPGSPLA | |
| | HHAMQNSLMSPCSPMRRGEVNMRYPSATRNYSGGVMGSWHMDASLDEGF | |
| | GSSLLEEFKSNKTRGFELSEIAGHVVEFSADQYGSRFIQQKLETATTDEKNM | |
| | VYEEIMPHALALMTDVFGNYVIQKFFEHGLPPQRRELGDKLFENVLPLSLQ | |
| | MYGCRVIQKAIEVVDLDQKIKMVKELDGHVMRCVRDQNGNHVVQKCIEC | |
| | VPEENIEFIISTFFGHVVSLSTHPYGCRVIQRVLEHCHDPDTQSKVMEEILSTV | |
| | SMLAQDQYGNYVVQHVLEHGKPDERTVIIKELAGKIVQMSQQKFASNVVE | |
| | KCLTFGGPEERELLVNEMLGTTDENEPLQAMMKDQFANYVVQKVLETCDD | |
| | QQRELILTRIKVHLNALKKYTYGKHVVARIEKLVAAGGMHMFLLFPLGLKE | |
| | ENGFAVPNPASDVVRPQVLYSLTRVDGSAIAF | |
| 103 | HRGNEDLSFGDDYEKEIGLLLGEQQRRQEEADEIEKELNLYRSGSAPPTVDG | PUM-HD |
| | SVNAAGGLFNGGGRGPFMEFGGGNKGNGFGGDDDELRKDPAYLSYYYAN | |
| | MKLNPRLPPPLMSREDLRVAQRLKGSSNVLGGVGDRRNVNESRSLFSMPPG | |
| | FDQMNEFEAEKTNASSSEWDANGLIGLPGLGLGGKQKSFADIFQPDMGHPV | |
| | SQQPSRPASRNAFDENVDSTNNQSPSASQGIGAPPPYSYAAVLGSSLSRNGTP | |
| | DPQAVARVPSPCLTPIGSGRVSSNDKRNTSNQSPFNGVTSGLNESSDLVNAL | |
| | SGMNLSGSGGLDDRGQAEQDVEKVRNYMFGFQGGHNEVSQHVFPNKSDQ | |
| | AQKATGSLRNLHMRGSQGSAYNGGGLANPYQHLDSPNYCLNNYALNPAV | |
| | ASVMANQLGNSNFSPMYDNYSAASALGFSGMDSRLHGGGFESRNLGRSNR | |
| | MMGGGGLQSHMADPMYHQYGRYSENVDALDLLNDPAMDRSFMGNSYMN | |
| | MLELQRAYLGAQKSQYGVPYKSGSPNSHSYYGSPTFGSNMSYPGSPLAHHA | |
| | MQNSLMSPCSPMRRGEVNMRYPSATRNYSGGVMGSWHMDASLDEGFGSS | |
| | LLEEFKSNKTRGFELSEIAGHVVEFSADQYGSRFIQQKLETATTDEKNMVYE | |
| | EIMPHALALMTDVFGNYVIQKFFEHGLPPQRRELGDKLFENVLPLSLQMYG | |
| | CRVIQKAIEVVDLDQKIKMVKELDGHVMRCVRDQNGNHVVQKCIECVPEE | |
| | NIEFIISTFFGHVVSLSTHPYGCRVIQRVLEHCHDPDTQSKVMEEILSTVSMLA | |
| | QDQYGNYVVQHVLEHGKPDERTVIIKELAGKIVQMSQQKFASNVVEKCLTF | |
| | GGPEERELLVNEMLGTTDENEPLQAMMKDQFANYVVQKVLETCDDQQRE | |
| | LILTRIKVHLNALKKYTYGKHVVARIEKLVAAGGMHMFLLFPLGLKEENGF | |
| | AVPNPASDVVRPQVLYSLTRVDGSAIAF | |

Polypeptides with Phase Behavior

In embodiments, the fusion proteins described herein comprise one or more polypeptides with phase behavior. In embodiments, the fusion proteins described herein comprise from 1 to 2, from 1 to 3, from 1 to 4, from 1 to 5, from 1 to 6, from 1 to 7, from 1 to 8, from 1 to 9, from 1 to 10, from 1 to 15, from 1 to 20, from 1 to 25, from 1 to 30, from 1 to 35, from 1 to 40, from 1 to 45, or from 1 to 50 polypeptides with phase behavior. In embodiments, the fusion proteins comprise 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 polypeptides with phase behavior.

In embodiments, the polypeptide with phase behavior is a resilin-like polypeptide (RLP). Resilin-like polypeptides are elastomeric polypeptides with mechanical properties including desirable resilience, compressive elastic modulus, tensile elastic modulus, shear modulus, extension to break, maximum tensile strength, hardness, rebound, and compression set. In embodiments, the resilin-like polypeptides described herein are polymers which comprise one or more repeats. In embodiments, the polymeric repeats may have an amino acid sequence selected from any one of SEQ ID NOS: 1-9.

In embodiments, a resilin-like polypeptide comprises more than one type of repeat, e.g. a repeat of SEQ ID NO: 1 and a repeat of SEQ ID NO: 3.

In embodiments, the resilin-like polypeptides described herein comprise repeats that occur up to 500 times within a given RLP. In embodiments, the repeats occur about 1, about 2, about 3, about 4, about 5, about 6, about 7, about 8, about 9, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 450, or about 500 times.

In embodiments, the RLP comprises one or more partial repeats. In embodiments, the length of a partial repeat is 1, 2, 3, 4, 5, 6, 7, 8, 9, 10, or more amino acids. In embodiments, the RLP comprises one or more additional amino acids at the N-terminus or C-terminus of the RLP that are not part of a repeat.

In embodiments, one or more RLP repeats are scrambled, i.e., they contain a different amino acid sequence but retain the same amino acid composition. For example, a repeat may have a different amino acid sequence than SEQ ID NO: 8, but retain the same amino acid composition. In embodiments, the polypeptide with phase behavior is an elastin-like polypeptide. Elastin-like polypeptides (ELPs) are biopolymers derived from tropoelastin. In embodiments, the polypeptide with phase behavior comprises a P and G motif, comprising a plurality of P residues and a plurality of G residues. In embodiments, the P and G motif comprises at least about 10% proline and at least about 20% glycine. In embodiments, the elastin-like polypeptides described herein are polymers comprising a pentapeptide repeat having the sequence (Val-Pro-Gly-Xaa-Gly)n (SEQ ID NO: 217). In embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500, including all values and ranges in between. In embodiments, n is an integer from 1 to 360, inclusive of endpoints. In embodiments, the polypeptide with phase behavior comprises an amino acid sequence selected from:

a.
(SEQ ID NO: 1)
(GRGDSPY)ₙ
b.
(SEQ ID NO: 2)
(GRGDSPH)ₙ
c.
(SEQ ID NO: 3)
(GRGDSPV)ₙ
d.
(SEQ ID NO: 4)
(GRGDSPYG)ₙ
e.
(SEQ ID NO: 5)
(RPLGYDS)ₙ
f.
(SEQ ID NO: 6)
(RPAGYDS)ₙ
g.
(SEQ ID NO: 7)
(GRGDSYP)ₙ
h.
(SEQ ID NO: 8)
(GRGDSPYQ)ₙ
i.
(SEQ ID NO: 9)
(GRGNSPYG)ₙ
j.
(SEQ ID NO: 10)
(GVGVP)ₙ;
k.
(SEQ ID NO: 11)
(GVGVPGLGVPGVGVPGLGVPGVGVP)ₘ;
l.
(SEQ ID NO: 12)
(GVGVPGVGVPGAGVPGVGVPGVGVP)ₘ;
m.
(SEQ ID NO: 13)
(GVGVPGWGVPGVGVPGWGVPGVGVP)ₘ;
n.
(SEQ ID NO: 14)
(GVGVPGVGVPGVGVPGVGVPGVGVPGVGVPGEGVPGFGVPGVGVP)ₘ;
o.
(SEQ ID NO: 15)
(GVGVPGVGVPGVGVPGVGVPGVGVPGVGVPGKGVPGFGVPGVGVP)ₘ;
and
p.
(SEQ ID NO: 16)
(GAGVPGVGVPGAGVPGVGVPGAGVP)ₘ;

or a randomized, scrambled analog thereof. In embodiments, n is an integer in the range of 1-500 (1, 20, 40, 60, 80, 100, 120, 140, 160, 180, 200, 220, 240, 260, 280, 300, 320, 340, 360, 380, 400, 420, 440, 460, 480, or 500, including any values or ranges therein). In embodiments, the n is an integer in the range of 20-360, inclusive of endpoints. In embodiments, m is an integer in the range of 1-100 (1, 10, 20, 30, 40, 50, 60, 70, 80, 90, or 100, including any values of ranges therein.) In embodiments, m is an integer in the range of 4-25, inclusive of endpoints.

In embodiments, the polypeptide with phase behavior comprises an amino acid sequence selected from: (GVGVP)ₘ (SEQ ID NO: 22); (ZZPXXXXGZ)ₘ (SEQ ID NO: 23); (ZZPXGZ)ₘ (SEQ ID NO: 24); (ZZPXXGZ)ₘ (SEQ ID NO: 25); or (ZZPXXXGZ)ₘ (SEQ ID NO: 26), wherein m is an integer between 10 and 160, inclusive of endpoints, wherein X if present is any amino acid except proline or glycine, and wherein Z if present is any amino acid. In embodiments, the polypeptide with phase behavior comprises an amino acid sequence selected from: (GVGVPGVGVPGAGVPGVGVPGVGVP)m (SEQ ID NO: 17); or (GVGVPGVGVPGLGVPGVGVPGVGVP)ₘ (SEQ ID NO: 18); wherein m is an integer between 2 and 32, inclusive of endpoints. the polypeptide with phase behavior comprises an amino acid sequence selected from: (GVGVPGVGVPGAGVPGVGVPGVGVP)ₘ (SEQ ID NO: 19), wherein m is 8 or 16; (GVGVPGAGVP)ₘ (SEQ ID NO: 20), wherein m is an integer between 5 and 80, inclusive of endpoints; or (GXGVP)ₘ (SEQ ID NO: 21), wherein m is an integer between 10 and 160, inclusive of endpoints, and wherein X for each repeat is independently selected from the group consisting of glycine, alanine, valine, isoleucine, leucine, phenylalanine, tyrosine, tryptophan, lysine, arginine, aspartic acid, glutamic acid, and serine. In embodiments, m is an integer in the range of 1-100 (1, 10, 20, 30, 40, 50, 60, 70, 80, 90, or 100, including any values of ranges therein.)

In embodiments, the pentapeptide repeat is scrambled, for example it comprises a different amino acid sequence but maintains the same amino acid composition. For example, an ELP may comprise a different amino acid sequence than SEQ ID NO: 217, but maintains the same amino acid composition, e.g. 40% of the sequence is glycine, 20% of the sequence is Xaa, 20% of the sequence is proline, and 20% of the sequence is valine.

In embodiments, the ELP comprises one or more partial repeats. In embodiments, the length of a partial repeat is 1, 2, 3, or 4 amino acids. In embodiments, the ELP comprises one or more additional amino acids at the N-terminus or C-terminus of the ELP that are not part of a repeat.

ELPs and RLPs undergo a phase transition in response to an environmental factor. ELPs and RLPs retain their ability to undergo a phase transition when coupled to one or more polypeptides (such as one or more NBP), or expressed as a fusion protein with one or more other polypeptides (such as one or more NBP). Polymers like ELPs and RLPs exhibit a transition temperature (Tt), also referred to as a cloud point temperature (Tc). In some embodiments ELPs and RLPs undergo a reversible phase transition from a soluble to an insoluble phase at the Tt. ELPs that transition from a soluble to an insoluble phase with heating or an increase in salt concentration have a Tt referred to as a lower critical solution temperature (LCST). RLPs that transition from a soluble to an insoluble phase with cooling or a decrease in salt concentration have a Tt referred to as a lower critical solution temperature (UCST). In embodiments, the phase transition results from a change in secondary structure of the ELP and/or RLP. For example, the phase transition of an ELP results from a change in secondary structure from a random coil (below the Tt) to a type II β-turn. In embodiments, the change in secondary structure is characterized by a method selected from circular dichroism spectropolarimetry, small angle x-ray scattering, and cryo-electron microscopy, ultraviolet-visible spectrophotometry, static light scattering, dynamic light scattering, nuclear magnetic resonance spectroscopy, solid-state nuclear magnetic resonance spectroscopy, infrared spectroscopy, Fourier transform infrared spectroscopy (FTIR), microscopy, and small angle neutron scattering. In embodiments, the phase transition of an ELP does not result from a chance in secondary structure.

In embodiments, the RLPs and ELPs described herein have a transition temperature between about 0° C. and about 100° C. In embodiments, the RLPs and ELPs described herein have a transition temperature between about 10° C. and about 50° C. In some embodiments the transition temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C. In embodiments, the RLPs described herein have a transition temperature from about 10° C. to about 100° C.

In embodiments, the Tt of the RLPs and ELPs described herein is modulated by manipulating the primary structure e.g. amino acid sequence of the RLP and ELP. In embodiments, the hydrophobicity of the ELP or RLP is modulated. In embodiments, the hydrophobicity of the ELP is modified by altering the identity of the guest residue Xaa. In embodiments, the hydrophobicity of the ELP or RLP is increased resulting in a decreased Tt. In embodiments, the hydrophobicity of the ELP or RLP is decreased resulting in an increased Tt. In embodiments, the polarity of the ELP or RLP is modulated. In embodiments, the polarity of the ELP is modulated by altering the identity of the guest residue Xaa. In embodiments, the polarity of the ELP or RLP is increased resulting in an increased Tt. In embodiments, the polarity of the ELP or RLP is decreased resulting in a decreased Tt.

In embodiments, the number of ELP pentapeptide repeats (n) is modulated to alter the Tt. In embodiments, n of the pentapeptide repeat (Val-Pro-Gly-Xaa-Gly)n (SEQ ID NO: 217) is an integer from 1 to 500, inclusive of endpoints. In embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500, including all values and ranges in between.

In embodiments, Xaa also referred to herein as “the guest residue” is any amino acid that does not eliminate the phase behavior of the ELP. In embodiments, Xaa is any amino acid except proline. In embodiments, Xaa is independently selected for each repeat. For example, a given ELP may contain the guest residues alanine, glycine, and valine at a ratio of 8:7:1. In some embodiments Xaa is selected from the group consisting of alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine and valine. In embodiments, Xaa is a non-classical amino acid selected from the group consisting of 2,4-diaminobutyric acid, α-amino-isobutyric acid, alloisoleucine, 4-aminobutyric acid, 2-amino butyric acid (Abu), ε-Ahx, 6-amino hexanoic acid, 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Na-methyl amino acids, and amino acid analogs in general. In embodiments, Xaa is the D-isomer of a natural or non-classical amino acid.

In embodiments, the Tt of the RLPs and ELPs described herein is modulated by introducing one or more environmental factors to the composition containing the RLP and/or ELP. In embodiments, the Tt of the ELPs and/or RLPs is modulated by adjusting the ionic strength of solvents. In embodiments, the ionic strength of the solvent is adjusted by adding salt. In embodiments, ELPs and/or RLPs contain lower Tt in solvents containing anions categorized as kosmotropes. Anions that are kosmotropes are highly hydrated and influence the water shield on ELPs and/or RLPs. In embodiments, the Tt of ELPs and/or RLPs can be adjusted through the addition of anions that are chaotropes. At low concentrations, the addition of chaotropes increase the Tt of the ELP and/or RLP. At high concentrations, the addition of a chaotrope decreases the Tt of the ELP and/or RLP. In embodiments, the Tt of the ELP and/or RLP can be tuned by introducing one or more reagents that disrupts hydrogen bonds. Non-limiting examples of reagents that disrupt hydrogen bonds include sodium dodecyl sulfate (SDS) and urea. In embodiments, reagents that enhance hydrogen bond formation are utilized to modulate the Tt. In embodiments, reagents that enhance hydrophobic interactions are utilized to modulate the Tt. Trifluoroethanol is a reagent which enhances both hydrophobic interactions and hydrogen bond formation, causing a decrease in Tt.

In embodiments, the ELP and/or RLP concentration can be adjusted to modulate Tt. In embodiments, a higher ELP and/or RLP concentration results in a reduced Tt. In embodiments, a lower ELP and/or RLP concentration results in an increased Tt.

In addition, modulation of pH, light, and ion concentrations also can be utilized to modulate Tt.

In embodiments, modulation of the number of (e.g. addition or removal) charged amino acids (e.g. histidine, lysine, arginine, glutamic acid, aspartic acid, ornithine, or other non-natural charged amino acids) and identity (e.g. positively or negatively charged) enables tuning of the Tt through pH modulation.

In embodiments, the ELPs and/or RLPs described herein are block copolymers. A block copolymer comprises two or more sequence domains or blocks, in which two or more blocks contain different properties. Non-limiting examples of properties that can be tuned include hydrophilicity, hydrophobicity, polarity, and secondary structure. In embodiments, the block copolymer is an amphiphile, e.g. it comprises at least one hydrophobic and at least one hydrophilic block.

In embodiments, the ELPs and/or RLPs described herein assemble into various morphologies. Non-limiting examples of morphologies include a spherical aggregate, a micelle, a vesicle, a fibril, a nanofibril, a nanotube, and a hydrogel. In embodiments, the RLPs and/or ELPs described herein assemble into various morphologies after the addition of an environmental factor. In embodiments, the RLPs and/or ELPs described herein change from one morphology to another morphology after the addition of an environmental factor. In embodiments, the RLPs and/or ELPs described herein change from one morphology to another morphology after the addition of a nucleic acid.

In embodiments, addition of an environmental factor causes an RLP and/or ELP to undergo a phase transition. In embodiments, at the RLP and/or ELP phase transition, the RLP and/or ELP converts from one morphology to another morphology.

In embodiments, a phase transition of an RLP and/or ELP causes the formation of dense, liquid, droplets.

In embodiments, the polypeptide with phase behavior comprises an amino acid sequence selected from Table 2. In embodiments, the polypeptide with phase behavior has at least 90% identity with a polypeptide of any one of SEQ ID NOs: 56, and 262-264. In embodiments, the polypeptide with phase behavior has at least 90% identity with a polypeptide of SEQ ID NO: 56. In embodiments, the polypeptide with phase behavior comprises any one of SEQ ID NOs: 56, and 262-264. In embodiments, the polypeptide with phase behavior comprises SEQ ID NO: 56.

TABLE 2
Exemplary amino acid sequences of polypeptides with phase behavior.

| SEQ ID | |
|---|---|
| NO | Sequence |
| 1 | (GRGDSPY)n, wherein n is an integer in the range of 1-500, inclusive of endpoints |
| 2 | (GRGDSPH)n, wherein n is an integer in the range of 1-500, inclusive of endpoints |
| 3 | (GRGDSPV)n, wherein n is an integer in the range of 1-500, inclusive of endpoints |
| 4 | (GRGDSPYG)n, wherein n is an integer in the range of 1-500, inclusive of endpoints |
| 5 | (RPLGYDS)n, wherein n is an integer in the range of 1-500, inclusive of endpoints |
| 6 | (RPAGYDS)n, wherein n is an integer in the range of 1-500, inclusive of endpoints |
| 7 | (GRGDSYP)n, wherein n is an integer in the range of 1-500, inclusive of endpoints |
| 8 | (GRGDSPYQ)n, wherein n is an integer in the range of 1-500, inclusive of endpoints |
| 9 | (GRGNSPYG)n, wherein n is an integer in the range of 1-500, inclusive of endpoints |
| 10 | (GVGVP)n, wherein n is an integer in the range of 1-500, inclusive of endpoints |
| 11 | (GVGVPGLGVPGVGVPGLGVPGVGVP)m, wherein m is an integer in the range of 4-25, |
| | inclusive of endpoints |
| 12 | (GVGVPGVGVPGAGVPGVGVPGVGVP)m, wherein m is an integer in the range of 4-25, |
| | inclusive of endpoints |
| 13 | (GVGVPGWGVPGVGVPGWGVPGVGVP)m, wherein m is an integer in the range of 4-25, |
| | inclusive of endpoints |
| 14 | (GVGVPGVGVPGVGVPGVGVPGVGVPGVGVPGEGVPGFGVPGVGVP)m, wherein m is |
| | an integer in the range of 4-25, inclusive of endpoints |
| 15 | (GVGVPGVGVPGVGVPGVGVPGVGVPGVGVPGKGVPGFGVPGVGVP)m, wherein m is |
| | an integer in the range of 4-25, inclusive of endpoints |
| 16 | (GAGVPGVGVPGAGVPGVGVPGAGVP)m, wherein m is an integer in the range of 4-25, |
| | inclusive of endpoints |
| 17 | (GVGVPGVGVPGAGVPGVGVPGVGVP)m, wherein m is an integer between 2 and 32, |
| | inclusive of endpoints |
| 18 | (GVGVPGVGVPGLGVPGVGVPGVGVP)m, wherein m is an integer between 2 and 32, |
| | inclusive of endpoints |
| 19 | (GVGVPGVGVPGAGVPGVGVPGVGVP)m, wherein m is 8 or 16 |
| 20 | (GVGVPGAGVP)m, wherein m is an integer between 5 and 80, inclusive of endpoints |
| 21 | (GXGVP)m, wherein m is an integer between 10 and 160, inclusive of endpoints, and |
| | wherein X for each repeat is independently selected from the group consisting of |
| | glycine, alanine, valine, isoleucine, leucine, phenylalanine, tyrosine, tryptophan, |
| | lysine, arginine, aspartic acid, glutamic acid, and serine. |
| 22 | (GVGVP)m, wherein m is an integer between 10 and 160, inclusive of endpoints |
| 23 | (ZZPXXXXGZ)m, wherein m is an integer between 10 and 160, inclusive of endpoints, |
| | wherein X if present is any amino acid except proline or glycine, and wherein Z if |
| | present is any amino acid |
| 24 | (ZZPXGZ)m, wherein m is an integer between 10 and 160, inclusive of endpoints, wherein |
| | X if present is any amino acid except proline or glycine, and wherein Z if present is |
| | any amino acid |
| 25 | (ZZPXXGZ)m, wherein m is an integer between 10 and 160, inclusive of endpoints, wherein |
| | X if present is any amino acid except proline or glycine, and wherein Z if present is |
| | any amino acid |
| 26 | (ZZPXXXGZ)m, wherein m is an integer between 10 and 160, inclusive of endpoints, |
| | wherein X if present is any amino acid except proline or glycine, and wherein Z if |
| | present is any amino acid |
| 27 | (GVGVP)m, wherein m is 20, 40, or 80 |
| 28 | (GRGDXPZX)m, wherein X is glutamine or serine, Z is tyrosine or valine, and m is an |
| | integer between 10 and 160, inclusive of endpoints |
| 29 | (XZPXDGRG)m, wherein X is glutamine or serine, Z is tyrosine or valine, and m is an |
| | integer between 10 and 160, inclusive of endpoints |
| 30 | (GRGDXPZX)40 |
| 31 | (GVGVP)80 |
| 32 | GRGDQPYQGRGDQPYQGRGDQPYQGRGDQPYQGRGDQPYQGRGDQPYQGRGDQPYQ |
| | GRGDQPYQGRGDQPYQGRGDQPYQGRGDQPYQGRGDQPYQGRGDQPYQGRGDQPYQ |
| | GRGDQPYQGRGDQPYQGRGDQPYQGRGDQPYQGRGDQPYQGRGDQPYQGRGDQPYQ |
| | GRGDQPYQGRGDQPYQGRGDQPYQGRGDQPYQGRGDQPYQGRGDQPYQGRGDQPYQ |
| | GRGDQPYQGRGDQPYQGRGDQPYQGRGDQPYQGRGDQPYQGRGDQPYQGRGDQPYQ |
| | GRGDQPYQGRGDQPYQGRGDQPYQGRGDQPYQGRGDQPYQGVGVPGVGVPGVGVPG |
| | VGVPGVGVPGVGVPGVGVPGVGVPGVGVPGVGVPGVGVPGVGVPGVGVPGVGVPGV |
| | GVPGVGVPGVGVPGVGVPGVGVPGVGVPGVGVPGVGVPGVGVPGVGVPGVGVPGVG |
| | VPGVGVPGVGVPGVGVPGVGVPGVGVPGVGVPGVGVPGVGVPGVGVPGVGVPGVGV |
| | PGVGVPGVGVPGVGVPGVGVPGVGVPGVGVPGVGVPGVGVPGVGVPGVGVPGVGVP |
| | GVGVPGVGVPGVGVPGVGVPGVGVPGVGVPGVGVPGVGVPGVGVPGVGVPGVGVPG |
| | VGVPGVGVPGVGVPGVGVPGVGVPGVGVPGVGVPGVGVPGVGVPGVGVPGVGVPGV |
| | GVPGVGVPGVGVPGVGVPGVGVPGVGVPGVGVPGVGVPGVGVPGVGVP |
| 33 | (GVGVPGLGVPGVGVPGLGVPGVGVP)m, wherein m is 16 |
| 34 | GVGVPGLGVPGVGVPGLGVPGVGVPGVGVPGLGVPGVGVPGLGVPGVGVPGVGVPGL |
| | GVPGVGVPGLGVPGVGVPGVGVPGLGVPGVGVPGLGVPGVGVPGVGVPGLGVPGVGV |
| | PGLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | P |
| 35 | GRGDSPYSGRGDSPYSGRGDSPYSGRGDSPYSGRGDSPYSGRGDSPYSGRGDSPYSGRG |
| | DSPYSGRGDSPYSGRGDSPYSGRGDSPYSGRGDSPYSGRGDSPYSGRGDSPYSGRGDSP |
| | YSGRGDSPYSGRGDSPYSGRGDSPYSGRGDSPYSGRGDSPYSGRGDSPYSGRGDSPYSG |
| | RGDSPYSGRGDSPYSGRGDSPYSGRGDSPYSGRGDSPYSGRGDSPYSGRGDSPYSGRGD |
| | SPYSGRGDSPYSGRGDSPYSGRGDSPYSGRGDSPYSGRGDSPYSGRGDSPYSGRGDSPY |
| | SGRGDSPYSGRGDSPYSGRGDSPYS |
| 36 | GVGVPGLGVPGVGVPGLGVPGVGVPGVGVPGLGVPGVGVPGLGVPGVGVPGVGVPGL |
| | GVPGVGVPGLGVPGVGVPGVGVPGLGVPGVGVPGLGVPGVGVPGVGVPGLGVPGVGV |
| | PGLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | PY |
| 37 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVPGVGVPGVGVPGAGVPGVGVPGVGV |
| | PGVGVPGVGVPGAGVPGVGVPGVGVPGVGVPGVGVPGAGVPGVGVPGVGVPGVGVP |
| | GVGVPGAGVPGVGVPGVGVPGVGVPGVGVPGAGVPGVGVPGVGVPGVGVPGVGVPG |
| | AGVPGVGVPGVGVPGVGVPGVGVPGAGVPGVGVPGVGVPGVGVPGVGVPGAGVPGV |
| | GVPGVGVPY |
| 38 | GVGVPOLOVPGVGVPGLGVPGVGVPGVGVPGLGVPGVGVPGLGVPGVGVPGVGVPGL |
| | GVPGVGVPGLGVPGVGVPGVGVPGLGVPGVGVPOLGVPGVGVPGVGVPGLGVPGVGV |
| | POLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLOVPGVGVPGVG |
| | VPGLGVPGVGVPGLGVPGVGVPGVGVPGLGVPGVGVPGLGVPGVGVPGVGVPGLGVP |
| | GVGVPOLOVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | P |
| 39 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVPGVGVPGVGVPGAGVPGVGVPGVGV |
| | PGVGVPGVGVPGAGVPGVGVPGVGVPGVGVPGVGVPGAGVPGVGVPGVGVPGVGVP |
| | GVGVPGAGVPGVGVPGVGVPGVGVPGVGVPGAGVPGVGVPGVGVPGVGVPGVGVPG |
| | AGVPGVGVPGVGVPGVGVPGVGVPGAGVPGVGVPGVGVPGVGVPGVGVPGAGVPGV |
| | GVPGVGVP |
| 40 | GVGPVGAGPVGVGPVGAGPVGVGPVGVGPVGAGPVGVGPVGAGPVGVGPVGVGPVG |
| | AGPVGVGPVGAGPVGVGPVGVGPVGAGPVGVGPVGAGPVGVGPVGVGPVGAGPVGV |
| | GPVGAGPVGVGPVGVGPVGAGPVGVGPVGAGPVGVGPVGVGPVGAGPVGVGPVGAG |
| | PVGVGPVGVGPVGAGPVGVGPVGAGPVGVGPVGVGPVGAGPVGVGPVGAGPVGVGP |
| | VGVGPVGAGPVGVGPVGAGPVGVGPVGVGPVGAGPVGVGPVGAGPVGVGPVGVGPV |
| | GAGPVGVGPVGAGPVGVGPVGVGPVGAGPVGVGPVGAGPVGVGPVGVGPVGAGPVG |
| | VGPVGAGPVGVGPV |
| 41 | GVGPVGAGPVGVGPVGAGPVGVGPVGVGPVGAGPVGVGPVGAGPVGVGPVGVGPVG |
| | AGPVGVGPVGAGPVGVGPVGVGPVGAGPVGVGPVGAGPVGVGPVGVGPVGAGPVGV |
| | GPVGAGPVGVGPVGVGPVGAGPVGVGPVGAGPVGVGPVGVGPVGAGPVGVGPVGAG |
| | PVGVGPVGVGPVGAGPVGVGPVGAGPVGVGPVGVGPVGAGPVGVGPVGAGPVGVGP |
| | VGVGPVGAGPVGVGPVGAGPVGVGPVGVGPVGAGPVGVGPVGAGPVGVGPVGVGPV |
| | GAGPVGVGPVGAGPVGVGPVGVGPVGAGPVGVGPVGAGPVGVGPVGVGPVGAGPVG |
| | VGPVGAGPVGVGPVGVGPVGAGPVGVGPVGAGPVGVGPVGVGPVGAGPVGVGPVGA |
| | GPVGVGPV |
| 42 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVPGVGVPGVGVPGAGVPGVGVPGVGV |
| | PGVGVPGVGVPGAGVPGVGVPGVGVPGVGVPGVGVPGAGVPGVGVPGVGVPGVGVP |
| | GVGVPGAGVPGVGVPGVGVPGVGVPGVGVPGAGVPGVGVPGVGVPGVGVPGVGVPG |
| | AGVPGVGVPGVGVPGVGVPGVGVPGAGVPGVGVPGVGVPGVGVPGVGVPGAGVPGV |
| | GVPGVGVP |
| 43 | GVGVPGLGVPGVGVPGLGVPGVGVPGVGVPGLGVPGVGVPGLGVPGVGVPGVGVPGL |
| | GVPGVGVPGLGVPGVGVPGVGVPGLGVPGVGVPGLGVPGVGVPGVGVPGLGVPGVGV |
| | PGLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | P |
| 44 | SKGPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | PGVGVPGLGVPGVGVPGLGVPGVGVPGVGVPGLGVPGVGVPGLGVPGVGVPGVGVPG |
| | LGVPGVGVPGLGVPGVGVPGVGVPGLGVPGVGVPGLGVPGVGVPGVGVPGLGVPGVG |
| | VPGLGVPGVGVPGVGVPGLGVPGVGVPGLGVPGVGVPGVGVPGLGVPGVGVPGLGVP |
| | GVGVP |
| 45 | SKGPGVGVPGVGVPGAGVPGVGVPGVGVPGVGVPGVGVPGAGVPGVGVPGVGVPGV |
| | GVPGVGVPGAGVPGVGVPGVGVPGVGVPGVGVPGAGVPGVGVPGVGVPGVGVPGVG |
| | VPGAGVPGVGVPGVGVPGVGVPGVGVPGAGVPGVGVPGVGVPGVGVPGVGVPGAGV |
| | PGVGVPGVGVPGVGVPGVGVPGAGVPGVGVPGVGVPGVGVPGVGVPGAGVPGVGVP |
| | GVGVPGVGVPGVGVPGAGVPGVGVPGVGVPGVGVPGVGVPGAGVPGVGVPGVGVPG |
| | VGVPGVGVPGAGVPGVGVPGVGVPGVGVPGVGVPGAGVPGVGVPGVGVPGVGVPGV |
| | GVPGAGVPGVGVPGVGVPGVGVPGVGVPGAGVPGVGVPGVGVPGVGVPGVGVPGAG |
| | VPGVGVPGVGVP |
| 46 | SKGPGVGPVGAGPVGVGPVGAGPVGVGPVGVGPVGAGPVGVGPVGAGPVGVGPVGV |
| | GPVGAGPVGVGPVGAGPVGVGPVGVGPVGAGPVGVGPVGAGPVGVGPVGVGPVGAG |
| | PVGVGPVGAGPVGVGPVGVGPVGAGPVGVGPVGAGPVGVGPVGVGPVGAGPVGVGP |
| | VGAGPVGVGPVGVGPVGAGPVGVGPVGAGPVGVGPVGVGPVGAGPVGVGPVGAGPV |
| | GVGPVGVGPVGAGPVGVGPVGAGPVGVGPVGVGPVGAGPVGVGPVGAGPVGVGPVG |
| | VGPVGAGPVGVGPVGAGPVGVGPVGVGPVGAGPVGVGPVGAGPVGVGPVGVGPVGA |
| | GPVGVGPVGAGPVGVGPV |
| 47 | GVGVPGLGVPGVGVPGLGVPGVGVPGVGVPGLGVPGVGVPGLGVPGVGVPGVGVPGL |
| | GVPGVGVPGLGVPGVGVPGVGVPGLGVPGVGVPGLGVPGVGVPGVGVPGLGVPGVGV |
| | PGLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | P |
| 48 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVPGVGVPGVGVPGAGVPGVGVPGVGV |
| | PGVGVPGVGVPGAGVPGVGVPGVGVPGVGVPGVGVPGAGVPGVGVPGVGVPGVGVP |
| | GVGVPGAGVPGVGVPGVGVPGVGVPGVGVPGAGVPGVGVPGVGVPGVGVPGVGVPG |
| | AGVPGVGVPGVGVPGVGVPGVGVPGAGVPGVGVPGVGVPGVGVPGVGVPGAGVPGV |
| | GVPGVGVP |
| 49 | GVGPVGAGPVGVGPVGAGPVGVGPVGVGPVGAGPVGVGPVGAGPVGVGPVGVGPVG |
| | AGPVGVGPVGAGPVGVGPVGVGPVGAGPVGVGPVGAGPVGVGPVGVGPVGAGPVGV |
| | GPVGAGPVGVGPVGVGPVGAGPVGVGPVGAGPVGVGPVGVGPVGAGPVGVGPVGAG |
| | PVGVGPVGVGPVGAGPVGVGPVGAGPVGVGPVGVGPVGAGPVGVGPVGAGPVGVGP |
| | VGVGPVGAGPVGVGPVGAGPVGVGPVGVGPVGAGPVGVGPVGAGPVGVGPVGVGPV |
| | GAGPVGVGPVGAGPVGVGPVGVGPVGAGPVGVGPVGAGPVGVGPVGVGPVGAGPVG |
| | VGPVGAGPVGVGPV |
| 50 | MSKGPGVGVPGLGVPGVGVPGLGVPGVGVPGVGVPGLGVPGVGVPGLGVPGVGVPGV |
| | GVPGLGVPGVGVPGLGVPGVGVPGVGVPGLGVPGVGVPGLGVPGVGVPGVGVPGLGV |
| | PGVGVPGLGVPGVGVPGVGVPGLGVPGVGVPGLGVPGVGVPGVGVPGLGVPGVGVPG |
| | LGVPGVGVPGVGVPGLGVPGVGVPGLGVPGVGVPGVGVPGLGVPGVGVPGLGVPGVG |
| | VPGVGVPGLGVPGVGVPGLGVPGVGVPGVGVPGLGVPGVGVPGLGVPGVGVPGVGVP |
| | GLGVPGVGVPGLGVPGVGVPGVGVPGLGVPGVGVPGLGVPGVGVPGVGVPGLGVPGV |
| | GVPGLGVPGVGVPGVGVPGLGVPGVGVPGLGVPGVGVPGVGVPGLGVPGVGVPGLGV |
| | PGVGVP |
| 51 | MSKGPGVGVPGVGVPGAGVPGVGVPGVGVPGVGVPGVGVPGAGVPGVGVPGVGVPG |
| | VGVPGVGVPGAGVPGVGVPGVGVPGVGVPGVGVPGAGVPGVGVPGVGVPGVGVPGV |
| | GVPGAGVPGVGVPGVGVPGVGVPGVGVPGAGVPGVGVPGVGVPGVGVPGVGVPGAG |
| | VPGVGVPGVGVPGVGVPGVGVPGAGVPGVGVPGVGVPGVGVPGVGVPGAGVPGVGV |
| | PGVGVPGVGVPGVGVPGAGVPGVGVPGVGVPGVGVPGVGVPGAGVPGVGVPGVGVP |
| | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVP |
| 52 | MSKGPGVGPVGAGPVGVGPVGAGPVGVGPVGVGPVGAGPVGVGPVGAGPVGVGPVG |
| | VGPVGAGPVGVGPVGAGPVGVGPVGVGPVGAGPVGVGPVGAGPVGVGPVGVGPVGA |
| | GPVGVGPVGAGPVGVGPVGVGPVGAGPVGVGPVGAGPVGVGPVGVGPVGAGPVGVG |
| | PVGAGPVGVGPVGVGPVGAGPVGVGPVGAGPVGVGPVGVGPVGAGPVGVGPVGAGP |
| | VGVGPVGVGPVGAGPVGVGPVGAGPVGVGPVGVGPVGAGPVGVGPVGAGPVGVGPV |
| | GVGPVGAGPVGVGPVGAGPVGVGPVGVGPVGAGPVGVGPVGAGPVGVGPVGVGPVG |
| | AGPVGVGPVGAGPVGVGPV |
| 53 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVPY |
| 54 | GVGVPGVGVPGAGVPGVGVPGVGVPGVGVPGVGVPGAGVPGVGVPGVGVPGVGVPG |
| | VGVPGAGVPGVGVPGVGVPGVGVPGVGVPGAGVPGVGVPGVGVPGVGVPGVGVPGA |
| | GVPGVGVPGVGVPGVGVPGVGVPGAGVPGVGVPGVGVPGVGVPGVGVPGAGVPGVG |
| | VPGVGVPGVGVPGVGVPGAGVPGVGVPGVGVP |
| 55 | GVGPVGAGPVGVGPVGAGPVGVGPVGVGPVGAGPVGVGPVGAGPVGVGPVGVGPVG |
| | AGPVGVGPVGAGPVGVGPVGVGPVGAGPVGVGPVGAGPVGVGPVGVGPVGAGPVGV |
| | GPVGAGPVGVGPVGVGPVGAGPVGVGPVGAGPVGVGPVGVGPVGAGPVGVGPVGAG |
| | PVGVGPVGVGPVGAGPVGVGPVGAGPVGVGPVGVGPVGAGPVGVGPVGAGPVGVGP |
| | VGVGPVGAGPVGVGPVGAGPVGVGPVGVGPVGAGPVGVGPVGAGPVGVGPVGVGPV |
| | GAGPVGVGPVGAGPVGVGPVGVGPVGAGPVGVGPVGAGPVGVGPVGVGPVGAGPVG |
| | VGPVGAGPVGVGPV |
| 56 | MSKGPGVGVPGVGVPGVGVPGVGVPGVGVPGVGVPGVGVPGVGVPGVGVPGVGVPG |
| | VGVPGVGVPGVGVPGVGVPGVGVPGVGVPGVGVPGVGVPGVGVPGVGVPGVGVPGV |
| | GVPGVGVPGVGVPGVGVPGVGVPGVGVPGVGVPGVGVPGVGVPGVGVPGVGVPGVG |
| | VPGVGVPGVGVPGVGVPGVGVPGVGVPGVGVPGVGVPGVGVPGVGVPGVGVPGVGV |
| | PGVGVPGVGVPGVGVPGVGVPGVGVPGVGVPGVGVPGVGVPGVGVPGVGVPGVGVP |
| | GVGVPGVGVPGVGVPGVGVPGVGVPGVGVPGVGVPGVGVPGVGVPGVGVPGVGVPG |
| | VGVPGVGVPGVGVPGVGVPGVGVPGVGVPGVGVPGVGVPGVGVPGVGVPGVGVPGV |
| | GVPGVGVPGVGVP |
| 57 | GVGVPGVGVPGVGVPGVGVPGVGVPGVGVPGVGVPGVGVPGVGVPGVGVPGVGVPG |
| | VGVPGVGVPGVGVPGVGVPGVGVPGVGVPGVGVPGVGVPGVGVPGVGVPGVGVPGV |
| | GVPGVGVPGVGVPGVGVPGVGVPGVGVPGVGVPGVGVPGVGVPGVGVPGVGVPGVG |
| | VPGVGVPGVGVPGVGVPGVGVPGVGVPGVGVPGVGVPGVGVPGVGVPGVGVPGVGV |
| | PGVGVPGVGVPGVGVPGVGVPGVGVPGVGVPGVGVPGVGVPGVGVPGVGVPGVGVP |
| | GVGVPGVGVPGVGVPGVGVPGVGVPGVGVPGVGVPGVGVPGVGVPGVGVPGVGVPG |
| | VGVPGVGVPGVGVPGVGVPGVGVPGVGVPGVGVPGVGVPGVGVPGVGVPGVGVPGV |
| | GVPGVGVP |
| 58 | MSKGPGVGVPGLGVPGVGVPGLGVPGVGVPGVGVPGLGVPGVGVPGLGVPGVGVPGV |
| | GVPGLGVPGVGVPGLGVPGVGVPGVGVPGLGVPGVGVPGLGVPGVGVPGVGVPGLGV |
| | PGVGVPGLGVPGVGVPGVGVPGLGVPGVGVPGLGVPGVGVPGVGVPGLGVPGVGVPG |
| | LGVPGVGVPGVGVPGLGVPGVGVPGLGVPGVGVPGVGVPGLGVPGVGVPGLGVPGVG |
| | VPGVGVPGLGVPGVGVPGLGVPGVGVPGVGVPGLGVPGVGVPGLGVPGVGVPGVGVP |
| | GLGVPGVGVPGLGVPGVGVPGVGVPGLGVPGVGVPGLGVPGVGVPGVGVPGLGVPGV |
| | GVPGLGVPGVGVPGVGVPGLGVPGVGVPGLGVPGVGVPGVGVPGLGVPGVGVPGLGV |
| | PGVGVPG |
| 59 | PGVGVPGLGVPGVGVPGLGVPGVGVPGVGVPGLGVPGVGVPGLGVPGVGVPGVGVPG |
| | LGVPGVGVPGLGVPGVGVPGVGVPGLGVPGVGVPGLGVPGVGVPGVGVPGLGVPGVG |
| | VPGLGVPGVGVPGVGVPGLGVPGVGVPGLGVPGVGVPGVGVPGLGVPGVGVPGLGVP |
| | GVGVPGVGVPGLGVPGVGVPGLGVPGVGVPGVGVPGLGVPGVGVPGLGVPGVGVPGV |
| | GVPGLGVPGVGVPGLGVPGVGVPGVGVPGLGVPGVGVPGLGVPGVGVPGVGVPGLGV |
| | PGVGVPGLGVPGVGVPGVGVPGLGVPGVGVPGLGVPGVGVPGVGVPGLGVPGVGVPG |
| | LGVPGVGVPGVGVPGLGVPGVGVPGLGVPGVGVPGVGVPGLGVPGVGVPGLGVPGVG |
| | VPG |
| 60 | GVGVPGLGVPGVGVPGLGVPGVGVPGVGVPGLGVPGVGVPGLGVPGVGVPGVGVPGL |
| | GVPGVGVPGLGVPGVGVPGVGVPGLGVPGVGVPGLGVPGVGVPGVGVPGLGVPGVGV |
| | PGLGVPGVGVPGVGVPGLGVPGVGVPGLGVPGVGVPGVGVPGLGVPGVGVPGLGVPG |
| | VGVPGVGVPGLGVPGVGVPGLGVPGVGVPGVGVPGLGVPGVGVPGLGVPGVGVPGVG |
| | VPGLGVPGVGVPGLGVPGVGVPGVGVPGLGVPGVGVPGLGVPGVGVPGVGVPGLGVP |
| | GVGVPGLGVPGVGVPGVGVPGLGVPGVGVPGLGVPGVGVPGVGVPGLGVPGVGVPGL |
| | GVPGVGVPGVGVPGLGVPGVGVPGLGVPGVGVPGVGVPGLGVPGVGVPGLGVPGVGV |
| | P |
| 217 | (Val-Pro-Gly-Xaa-Gly)n, wherein n is an integer in the range of 1-500, inclusive of |
| | endpoints |
| 262 | MGVGVPGVGVPGAGVPGVGVPGVGVPGVGVPGVGVPGAGVPGVGVPGVGVPGVGVP |
| | GVGVPGAGVPGVGVPGVGVPGVGVPGVGVPGAGVPGVGVPGVGVPGVGVPGVGVPG |
| | AGVPGVGVPGVGVPGVGVPGVGVPGAGVPGVGVPGVGVPGVGVPGVGVPGAGVPGV |
| | GVPGVGVPGVGVPGVGVPGAGVPGVGVPGVGVPGVGVPGVGVPGAGVPGVGVPGVG |
| | VPGVGVPGVGVPGAGVPGVGVPGVGVPGVGVPGVGVPGAGVPGVGVPGVGVPGVGV |
| | PGVGVPGAGVPGVGVPGVGVPGVGVPGVGVPGAGVPGVGVPGVGVPGVGVPGVGVP |
| | GAGVPGVGVPGVGVPGVGVPGVGVPGAGVPGVGVPGVGVPGVGVPGVGVPGAGVPG |
| | VGVPGVGVPGY |
| 263 | MGAGVPGVGVPGAGVPGVGVPGAGVPGVGVPGAGVPGVGVPGAGVPGVGVPGAGVP |
| | GVGVPGAGVPGVGVPGAGVPGVGVPGAGVPGVGVPGAGVPGVGVPGAGVPGVGVPG |
| | AGVPGVGVPGAGVPGVGVPGAGVPGVGVPGAGVPGVGVPGAGVPGVGVPGAGVPGV |
| | GVPGAGVPGVGVPGAGVPGVGVPGAGVPGVGVPGAGVPGVGVPGGAGVPGVGVPGA |
| | GVPGVGVPGAGVPGVGVPGAGVPGVGVPGAGVPGVGVPGAGVPGVGVPGAGVPGVG |
| | VPGAGVPGVGVPGAGVPGVGVPGAGVPGVGVPGAGVPGVGVPGAGVPGVGVPGAGV |
| | PGVGVPGAGVPGVGVPGAGVPGVGVPGAGVPGVGVPGAGVPGVGVPGAGVPGVGVP |
| | GAGVPGVGVPGWP |
| 264 | MSKGPGGVGVPGLGVPGVGVPGLGVPGVGVPGVGVPGLGVPGVGVPGLGVPGVGVPG |
| | VGVPGLGVPGVGVPGLGVPGVGVPGVGVPGLGVPGVGVPGLGVPGVGVPGVGVPGLG |
| | VPGVGVPGLGVPGVGVPGVGVPGLGVPGVGVPGLGVPGVGVPGVGVPGLGVPGVGVP |
| | GLGVPGVGVPGVGVPGLGVPGVGVPGLGVPGVGVPGVGVPGLGVPGVGVPGLGVPGV |
| | GVPGVGVPGLGVPGVGVPGLGVPGVGVPGVGVPGLGVPGVGVPGLGVPGVGVPGVGV |
| | PGLGVPGVGVPGLGVPGVGVPGVGVPGLGVPGVGVPGLGVPGVGVPGVGVPGLGVPG |
| | VGVPGLGVPGVGVPGVGVPGLGVPGVGVPGLGVPGVGVPGVGVPGLGVPGVGVPGLG |
| | VPGVGVPG |

In embodiments, the fusion protein comprises from 1 to 500, from 1 to 450, from 1 to 400, from 1 to 350, from 1 to 300, from 1 to 250, from 1 to 200 from 1 to 150, from 1 to 100, from 1 to 95, from 1 to 90, from 1 to 85, from 1 to 80, from 1 to 75, from 1 to 70, from 1 to 65, from 1 to 60, from 1 to 55, from 1 to 50, from 1 to 45, from 1 to 40, from 1 to 35, from 1 to 30, from 1 to 25, from 1 to 20, from 1 to 15, from 1 to 10, from 1 to 9, from 1 to 8, from 1 to 7, from 1 to 6, from 1 to 5, from 1 to 4, from 1 to 3, from 1 to 2, from 2 to 10, from 2 to 9, from 2 to 8, from 2 to 7, from 2 to 6, from 2 to 5, from 2 to 4, from 2 to 3, from 3 to 10, from 4 to 10, from 5 to 10, from 6 to 10, from 7 to 10, or from 8 to 10 different polypeptides with phase behavior. In embodiments, the fusion protein comprises at least one, at least two, at least three, at least four, at least five, at least six, at least seven, at least eight, at least nine, or at least ten different polypeptides with phase behavior. For example, the fusion protein may comprise a first polypeptide with phase behavior and a second polypeptide with phase behavior. In embodiments, the fusion protein comprises a third, fourth, fifth, sixth, seventh, eighth, ninth, or tenth polypeptide with phase behavior.

In embodiments, the fusion protein comprising an amino acid sequence selected from any one of SEQ ID NOs: 1-60 and 217 also comprises up to 10, up to 15, up to 20, or up to 25 additional N-terminal and/or C-terminal amino acids. In embodiments, the fusion protein comprising an amino acid sequence of any one of SEQ ID NOs: 1-60 and 217 also comprises an additional N-terminal methionine. In embodiments, the fusion protein comprising an amino acid sequence of any one of SEQ ID NOs: 1-60 and 217 also comprises an additional C-terminal glycine. In embodiments, the fusion protein comprising an amino acid sequence selected from any one of SEQ ID NOs: 56, and 262-264 also comprises up to 10, up to 15, up to 20, or up to 25 additional N-terminal and/or C-terminal amino acids. In embodiments, the fusion protein comprising an amino acid sequence of any one of SEQ ID NOs: 56, and 262-264 also comprises an additional N-terminal methionine. In embodiments, the fusion protein comprising an amino acid sequence of any one of SEQ ID NOs: 56, and 262-264 also comprises an additional C-terminal glycine. In embodiments, the fusion protein comprising an amino acid sequence selected of SEQ ID NOs: 56 also comprises up to 10, up to 15, up to 20, or up to 25 additional N-terminal and/or C-terminal amino acids. In embodiments, the fusion protein comprising an amino acid sequence of SEQ ID NOs: 56 also comprises an additional N-terminal methionine. In embodiments, the fusion protein comprising an amino acid sequence of SEQ ID NOs: 56 also comprises an additional C-terminal glycine.

In embodiments, a fusion protein contains polypeptide repeat units. In embodiments, there are from 5-500 polypeptide repeat units, including all ranges and values therebetween. In embodiments, there are 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 polypeptide repeat units.

In embodiments, the fusion protein has the same amino acid composition of an ELP and/or RLP but does not contain repeats. In embodiments, the fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an ELP and/or RLP. In embodiments, the fusion protein comprises an amino acid composition that is 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an ELP and/or RLP. In embodiments, the fusion protein comprises a composition of hydrophobic amino acids that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an ELP and/or RLP.

In embodiments, polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 1-60, 217, or 262-264. In embodiments, polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 56, and 262-264. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 56.

In embodiments, the polypeptide with phase behavior comprises a non-repetitive unstructured polypeptide. In embodiments, the non-repetitive unstructured polypeptide has an amino acid sequence that contains at least 50 amino acids. In embodiments, the non-repetitive unstructured polypeptide has an amino acid sequence that contains at least 50, at least 60, at least 70, at least 80, at least 90, or at least 100 amino acids. In embodiments, the polypeptide with phase behavior comprises a P and G motif, comprising a plurality of P residues and a plurality of G residues. In embodiments, the P and G motif comprises at least about 10% proline and at least about 20% glycine. In embodiments, the sequence of the non-repetitive unstructured polypeptide is at least about 10% proline (e.g. at least 10%, 20%, 30%, 40%, 50%, 60%, 70%, or 80%) and at least 20% glycine (e.g. at least 20%, at least 30%, at least 40%, at least 50%, at least 60%, at least 70%, at least 80%, or at least 90%). In embodiments, the non-repetitive unstructured polypeptide has a sequence that contains at least about 40% of amino acids selected from the group consisting of valine, alanine, leucine, lysine, threonine, isoleucine, tyrosine, serine, and phenylalanine.

In embodiments, the non-repeated unstructured polypeptide comprises a sequence that does not contain three contiguous identical amino acids, wherein any 5-10 amino acid subsequence does not occur more than once in the non-repeated unstructured polypeptide, and wherein when the non-repeated unstructured polypeptide comprises a subsequence starting and ending with proline, and wherein the subsequence further comprises at least one glycine.

In embodiments, the ELPs and/or RLPs described herein are expressed as a component of a polypeptide with phase behavior. In embodiments, the polypeptide with phase behavior is expressed in bacteria or mammalian cells. In embodiments, the polypeptide with phase behavior is expressed in Escherichia coli. In embodiments, the polypeptide with phase behavior is expressed in insect cells (e.g., Sf9 cells). In embodiments, the sequence of the non-repetitive unstructured polypeptide is at least about 10% proline (e.g. at least 10%, 20%, 30%, 40%) and at least 20% glycine (e.g. at least 20%, 30%, 40%, or 50%), and at least 40% (e.g. at least 40%, 50%, 60%, or 70%) of amino acids selected from the group consisting of valine, alanine, leucine, lysine, threonine, isoleucine, tyrosine, serine, and phenylalanine.

In embodiments, the ELPs and/or RLPs described herein are expressed as a component of a fusion protein. In embodiments, the fusion protein is expressed in bacteria or mammalian cells. In embodiments, the fusion protein is expressed in Escherichia coli. In embodiments, the fusion protein is expressed in insect cells (e.g., Sf9 cells).

In embodiments, the non-repetitive unstructured polypeptide does not contain three contiguous identical amino acids. In embodiments, the non-repetitive unstructured polypeptide comprises a subsequence (e.g. a fragment of the non-repetitive unstructured polypeptide) which only occurs once in the non-repetitive unstructured polypeptide sequence. In embodiments, the non-repetitive unstructured polypeptide comprises a subsequence that starts and ends with proline. In embodiments, the non-repetitive unstructured polypeptide comprises a subsequence that contains at least one glycine.

In embodiments, the polypeptide with phase behavior comprises a signal peptide. In embodiments, a signal peptides comprises an amino acid sequence selected from any one or SEQ ID NOS: 218-220. In embodiments, a signal peptides comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% identical to any one of SEQ ID NOS: 218-220. In embodiments, the signal peptide is a polypeptide from Table 3.

TABLE 3
Exemplary amino acid sequences of signal
peptides

| SEQ ID NO | SEQUENCE |
|---|---|
| 218 | SKGP |
| 219 | MSKP |
| 220 | TCSQDEFRCHDGKCISRQFVCDSDRDCLDGSDEASCPGY |

In embodiments, the polypeptide with phase behavior comprises an amino acid sequence of (GVGVPGLGVPGVGVPGLGVPGVGVP)m (SEQ ID NO: 33), wherein m is 16. In embodiments, the polypeptide with phase behavior comprises an amino acid sequence of SEQ ID NO: 34. In embodiments, the fusion protein comprises an amino acid sequence of SEQ ID NO: 38. In embodiments, the fusion protein comprises an amino acid sequence of SEQ ID NO: 43. In embodiments, the fusion protein comprises an amino acid sequence of SEQ ID NO: 47. In embodiments, the fusion protein comprises an amino acid sequence of SEQ ID NO: 60.

Linkers Between Polypeptides with Phase Behavior and NBPs

In embodiments, the fusion protein comprises a linker. In embodiments, the NBP is coupled to the polypeptide with phase behavior via a linker. In embodiments, any linker that does not interfere with the function of the fusion protein may be utilized. In embodiments, a fusion comprises, from C-terminus to N-terminus one or more NBP, one or more linkers, and one or more polypeptides with phase behavior. In embodiments, a fusion comprises, from N-terminus to C-terminus one or more NBP, one or more linkers, and one or more polypeptides with phase behavior.

In embodiments, the linker connects the NBP to the polypeptide with phase behavior. In embodiments, the linker enables cooperative interactions between the polypeptide with phase behavior and the NBP. In embodiments, the linker is a peptide. In embodiments, the linker preserves the phase behavior of the polypeptide with phase behavior. In embodiments, the linker preserves the Tₜ of the polypeptide with phase behavior. In embodiments, the linker preserves the structure of the NBP. In embodiments, the linker comprises between 1 and 50 amino acids. In embodiments, the linker comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In embodiments, the stiffness of the linker is increased by the inclusion of proline in the linker amino acid sequence.

In embodiments, the flexibility of a linker is increased by the inclusion of small polar amino acids, including threonine, serine, and glycine.

In embodiments, the linker may adopt various secondary structures, including but not limited to α-helices, β-strands, and random coils. In embodiments, the linker adopts an α-helix and comprises an amino acid repeat of (EAAAK)ₙ (SEQ ID NO: 143) where n is a repeat number, i.e., an integer in the range of 1 to 20, inclusive of endpoints.

In embodiments, the linker is comprised of (G₄S)ₙ(SEQ ID NO: 144) where n can be an integer from 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). In embodiments, the polypeptide linker has a repeat of (SGGG)n (SEQ ID NO: 145), wherein n is an integer from 1 to 50 (e.g. 1, 2, 3, 4, 5, 6, 7, 8, 9, 10, 11, 12, 13, 14, 15, 16, 17, 18, 19, 20). In embodiments, the polypeptide linker has a repeat of (GGGS)n (SEQ ID NO: 146), wherein n is an integer from 1 to 20 (e.g. 1, 2, 3, 4, 5, 6, 7, 8, 9, 10, 11, 12, 13, 14, 15, 16, 17, 18, 19, or 20).

In embodiments, the linker has an amino acid sequence of KESGSVSSEQLAQFRSLD (SEQ ID NO: 147). In embodiments, the linker has an amino acid sequence of EGKSSGSGSESKST (SEQ ID NO: 148). In embodiments, the linker only contains glycine.

In embodiments, the peptide linker comprises a protease cleavage site. In embodiments, the protease cleavage site is a furin cleavage site.

In embodiments, the polypeptide linker is a poly-(Gly)ₙ linker, wherein n is an integer from 1 to 30 (e.g., 1, 2, 3, 4, 5, 6, 7, 8, 9, 10, 15, 16, 17, 18, 19, 20, 21, 22, 23, 24, 25, 26, 27, 28, 29, or 30 (SEQ ID NO: 149). In other embodiments, the linker is selected from the group consisting of: dipeptides, tripeptides, and quadripeptides. In embodiments, the linker is a dipeptide selected from the group consisting of alanine-serine (AS), leucine-glutamic acid (LE), and serine-arginine (SR).

In embodiments, the linker is selected from GKSSGSGSESKS (SEQ ID NO: 150), GSTSGSGKSSEGKG (SEQ ID NO: 151), GSTSGSGKSSEGSGSTKG (SEQ ID NO: 152), GSTSGSGKPGSGEGSTKG (SEQ ID NO: 153), EGKSSGSGSESKEF (SEQ ID NO: 154), SRSSG (SEQ ID NO: 155), and SGSSC (SEQ ID NO: 156).

In embodiments, the linker is a self-cleaving peptide. In embodiments, the self-cleaving peptide is a 2A peptide. 2A peptides are a class of 18-22 amino acid long peptides that induce ribosomal skipping during translation of a protein in a cell. In embodiments, the 2A peptide is a T2A peptide having an amino acid sequence of EGRGSLLTCGDVEENPGP (SEQ ID NO: 157), a P2A peptide having an amino acid sequence of ATNFSLLKQAGDVEENPGP (SEQ ID NO: 158), an E2A peptide having an amino acid sequence of QCTNYALLKLAGDVESNPGP (SEQ ID NO: 159), or an F2A peptide having an amino acid sequence of VKQTLNFDLLKLAGDVESNPGP (SEQ ID NO: 160). In embodiments, the 2A peptide has at least 80%, at least 85%, at least 90%, at least 95%, or at least 98% identity to any one of SEQ ID NOs. 157-160. In embodiments, a 2A peptide further comprises GSG on its N-terminus (SEQ ID NOs: 161-164).

In embodiments, the linker comprises an amino acid sequence of any one of SEQ ID NOs: 143-216. In embodiments, the linker has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity to an amino acid sequence selected from any one of SEQ ID NOs: 143-216. In embodiments, the linker has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity to an amino acid sequence selected from any one of SEQ ID NOs: 143-216 and 261. In embodiments, the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 261.

In embodiments, the linker is a polypeptide from Table 4.

TABLE 4
Exemplary amino acid sequences of linker sequences

| SEQ ID NO | SEQUENCE |
|---|---|
| 143 | (EAAAK)ₙ, wherein n is an integer in the range of 1-20, inclusive of endpoints |
| 144 | (G4S)ₙ, wherein n is an integer in the range of 1-30, inclusive of endpoints |
| 145 | (SGGG)ₙ, wherein n is an integer in the range of 1-50, inclusive of endpoints |
| 146 | (GGGS)ₙ, wherein n is an integer in the range of 1-20, inclusive of endpoints |
| 147 | KESGSVSSEQLAQFRSLD |
| 148 | EGKSSGSGSESKST |
| 149 | poly-(Gly)ₙ, wherein n is an integer in the range of 1-30, inclusive of endpoints |
| 150 | GKSSGSGSESKS |
| 151 | GSTSGSGKSSEGKG |
| 152 | GSTSGSGKSSEGSGSTKG |
| 153 | GSTSGSGKPGSGEGSTKG |
| 154 | EGKSSGSGSESKEF |
| 155 | SRSSG |
| 156 | SGSSC |
| 157 | EGRGSLLTCGDVEENPGP |
| 158 | ATNFSLLKQAGDVEENPGP |
| 159 | QCTNYALLKLAGDVESNPGP |
| 160 | VKQTLNFDLLKLAGDVESNPGP |
| 161 | GSGEGRGSLLTCGDVEENPGP |
| 162 | GSGATNFSLLKQAGDVEENPGP |
| 163 | GSGQCTNYALLKLAGDVESNPGP |
| 164 | GSGVKQTLNFDLLKLAGDVESNPGP |
| 165 | GGGGSGGSGGGGSGGRGS |
| 166 | AAAAA |
| 167 | AAAAAAAAAA |
| 168 | AAAAAAGGGGG |
| 169 | AGAGA |
| 170 | EKAREK |
| 171 | GAAGA |
| 172 | GASGAS |
| 173 | GEGEF |
| 174 | GGGGS |
| 175 | GGGGSGGGG |
| 176 | GGGGSGGGGS |
| 177 | GGGGSGGGGSGGGG |
| 178 | GGGGSGGGGSGGGGS |
| 179 | GGGGSGGGGSGGGGSGGGGS |
| 180 | GGGGSGGGGSGGGGSGGGGSGGGGS |
| 181 | GGGSGGGSGGG |
| 182 | GGSGGSGG |
| 183 | GGSGGS |
| 184 | GGSGGSGGS |
| 185 | GGSGGSGGSGGS |
| 186 | GPGGPG |
| 187 | GPGPG |
| 188 | GPGPGP |
| 189 | GPPGPP |
| 190 | GPSGPG |
| 191 | GPSGPS |
| 192 | GPSGSGPG |
| 193 | GSGGGGSGGGGSGGGGSGS |
| 194 | GSGGGGSGGGGSGS |
| 195 | GSGGGGSGS |
| 196 | GSGGGSGGGSGGGSGS |
| 197 | GSGS |
| 198 | GSGSAGSGSA |
| 199 | GSGSGS |
| 200 | GSGSGSGSG |
| 201 | GSSG |
| 202 | GSSGGGGS |
| 203 | KKKKKGGGGG |
| 204 | PAPAP |
| 205 | PGPGPGPGP |
| 206 | QPGSGP |
| 207 | SGAGSAGA |
| 208 | SGGSGG |
| 209 | SGGSGGSGG |
| 210 | SGGSGGSGGSGG |
| 211 | SGSGGSGGSGGSGG |
| 212 | SGSGSGSG |
| 213 | SSGSSGSS |
| 214 | TGTGGSGSGTG |
| 215 | TGTGGTGTGG |
| 216 | YPKYVKQNTLKLAT |
| 261 | GYGGGSASG |

In embodiments, the linker is a chemical linker. In embodiments, the chemical linker is selected from the group consisting of a carbohydrate linker, a lipid linker, a fatty acid linker, and a polyether linker.

In embodiments, the linker is a direct covalent linkage between an amino acid residue of the polypeptide with an amino acid residue of the polypeptide with phase behavior and a NBP. In embodiments, a fusion protein comprises the polypeptide with phase behavior and a NBP. In embodiments, an amino acid residue of the polypeptide with phase behavior is covalently linked to an amino acid in the Bs-CspB. In embodiments, the fusion protein further comprises one or more linkers as described herein. In embodiments, a fusion protein comprises, from N-terminus to C-terminus, a polypeptide with phase behavior, a linker, and a NBP. In embodiments, a fusion protein comprises, from N-terminus to C-terminus, a NBP, a linker, and a polypeptide with phase behavior. In embodiments, a fusion protein comprises, from C-terminus to N-terminus, a polypeptide with phase behavior, a linker, and a NBP. In embodiments, a fusion protein comprises, from C-terminus to N-terminus, a NBP, a linker, and a polypeptide with phase behavior. In embodiments, a fusion protein comprises, from N-terminus to C-terminus, one or more polypeptides with phase behavior, one or more linkers, and one or more NBPs. In embodiments, a fusion protein comprises, from N-terminus to C-terminus, one or more NBPs, one or more linkers, and one or more polypeptides with phase behavior.

Fusion Protein Construct Comprising Bs-CspB and a Polypeptide with Phase Behavior

In some embodiments, the fusion protein described herein comprises a Bs-CspB and a polypeptide with phase behavior. In embodiments, a fusion protein comprises, from N-terminus to C-terminus, a polypeptide with phase behavior, a linker, and a Bs-CspB. In embodiments, a fusion protein comprises, from N-terminus to C-terminus, a Bs-CspB, a linker, and a polypeptide with phase behavior. In embodiments, a fusion protein comprises, from C-terminus to N-terminus, a polypeptide with phase behavior, a linker, and a Bs-CspB. In embodiments, a fusion protein comprises, from C-terminus to N-terminus, a Bs-CspB, a linker, and a polypeptide with phase behavior.

In embodiments, the Bs-CspB has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 90. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 56, and 262-264. In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 56.

In embodiments, the fusion protein comprises at least 90% identity with a polypeptide of SEQ ID NO: 90 and at least 90% identity to a polypeptide of any one of SEQ ID NOs: 56, and 262-264. In embodiments, the fusion protein comprises at least 90% identity with a polypeptide of SEQ ID NOs: 90 and at least 90% identity with a polypeptide of SEQ ID NO: 56. In embodiments, the fusion protein comprises SEQ ID NO: 90 and any one of SEQ ID NOs: 56, 262-264. In embodiments, the fusion protein comprises SEQ ID NOs: 90 and SEQ ID NO: 56.

In embodiments, the fusion protein further comprises a linker. In embodiments, the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 261.

In embodiments, the fusion protein has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 225 or 226. In embodiments, the fusion protein has at least 90% identity with a polypeptide of SEQ ID NO: 225 or 226. In embodiments, the fusion protein has at least 90% identity with a polypeptide of SEQ ID NO: 225. In embodiments, the fusion protein comprises SEQ ID NO: 225 or 226. In embodiments, the fusion protein comprises SEQ ID NO: 225. In embodiments, the fusion protein comprises a start codon. In embodiments, the start codon comprises a N-terminal methionine. In embodiments, the fusion protein comprises a N-terminal methionine. In embodiments, the fusion protein comprising a N-terminal methionine has an amino acid sequence of SEQ ID NO: 225. In embodiments, the fusion protein that does not comprise a N-terminal methionine has an amino acid sequence of SEQ ID NO: 226.

Exemplary sequences for the fusion protein described above are in Table 5.

TABLE 5
Amino acid sequences of a fusion protein comprising Bs-CspB and a polypeptide with
phase behavior.

| SEQ | | |
|---|---|---|
| ID | | |
| NO | ID | Sequence |
| 90 | Bs-CspB | LEGKVKWFNSEKGFGFIEVEGQDDVFVHFSAIQGEGFKTLEEGQAVSFEIVEGNR |
| | | GPQAANVTKEA |
| 56 | Polypeptide | MSKGPGVGVPGVGVPGVGVPGVGVPGVGVPGVGVPGVGVPGVGVPGVGVPGVG |
| | with phase | VPGVGVPGVGVPGVGVPGVGVPGVGVPGVGVPGVGVPGVGVPGVGVPGVGVPG |
| | behavior | VGVPGVGVPGVGVPGVGVPGVGVPGVGVPGVGVPGVGVPGVGVPGVGVPGVGV |
| | (100V80) | PGVGVPGVGVPGVGVPGVGVPGVGVPGVGVPGVGVPGVGVPGVGVPGVGVPGV |
| | | GVPGVGVPGVGVPGVGVPGVGVPGVGVPGVGVPGVGVPGVGVPGVGVPGVGVP |
| | | GVGVPGVGVPGVGVPGVGVPGVGVPGVGVPGVGVPGVGVPGVGVPGVGVPGVG |
| | | VPGVGVPGVGVPGVGVPGVGVPGVGVPGVGVPGVGVPGVGVPGVGVPGVGVPG |
| | | VGVPGVGVPGVGVPGVGVPGVGVPGVGVPGVGVP |
| 262 | Polypeptide | MGVGVPGVGVPGAGVPGVGVPGVGVPGVGVPGVGVPGAGVPGVGVPGVGVPG |
| | with phase | VGVPGVGVPGAGVPGVGVPGVGVPGVGVPGVGVPGAGVPGVGVPGVGVPGVG |
| | behavior | VPGVGVPGAGVPGVGVPGVGVPGVGVPGVGVPGAGVPGVGVPGVGVPGVGVP |
| | (20A80) | GVGVPGAGVPGVGVPGVGVPGVGVPGVGVPGAGVPGVGVPGVGVPGVGVPGV |
| | | GVPGAGVPGVGVPGVGVPGVGVPGVGVPGAGVPGVGVPGVGVPGVGVPGVGV |
| | | PGAGVPGVGVPGVGVPGVGVPGVGVPGAGVPGVGVPGVGVPGVGVPGVGVPG |
| | | AGVPGVGVPGVGVPGVGVPGVGVPGAGVPGVGVPGVGVPGVGVPGVGVPGAG |
| | | VPGVGVPGVGVPGVGVPGVGVPGAGVPGVGVPGVGVPGY |
| 263 | Polypeptide | MGAGVPGVGVPGAGVPGVGVPGAGVPGVGVPGAGVPGVGVPGAGVPGVGVPG |
| | with phase | AGVPGVGVPGAGVPGVGVPGAGVPGVGVPGAGVPGVGVPGAGVPGVGVPGAG |
| | behavior | VPGVGVPGAGVPGVGVPGAGVPGVGVPGAGVPGVGVPGAGVPGVGVPGAGVP |
| | (50A80) | GVGVPGAGVPGVGVPGAGVPGVGVPGAGVPGVGVPGAGVPGVGVPGAGVPGV |
| | | GVPGGAGVPGVGVPGAGVPGVGVPGAGVPGVGVPGAGVPGVGVPGAGVPGVG |
| | | VPGAGVPGVGVPGAGVPGVGVPGAGVPGVGVPGAGVPGVGVPGAGVPGVGVP |
| | | GAGVPGVGVPGAGVPGVGVPGAGVPGVGVPGAGVPGVGVPGAGVPGVGVPGA |
| | | GVPGVGVPGAGVPGVGVPGAGVPGVGVPGAGVPGVGVPGWP |
| 264 | Polypeptide | MSKGPGGVGVPGLGVPGVGVPGLGVPGVGVPGVGVPGLGVPGVGVPGLGVPGV |
| | with phase | GVPGVGVPGLGVPGVGVPGLGVPGVGVPGVGVPGLGVPGVGVPGLGVPGVGVP |
| | behavior | GVGVPGLGVPGVGVPGLGVPGVGVPGVGVPGLGVPGVGVPGLGVPGVGVPGVG |
| | (40L80) | VPGLGVPGVGVPGLGVPGVGVPGVGVPGLGVPGVGVPGLGVPGVGVPGVGVPG |
| | | LGVPGVGVPGLGVPGVGVPGVGVPGLGVPGVGVPGLGVPGVGVPGVGVPGLGV |
| | | PGVGVPGLGVPGVGVPGVGVPGLGVPGVGVPGLGVPGVGVPGVGVPGLGVPGV |
| | | GVPGLGVPGVGVPGVGVPGLGVPGVGVPGLGVPGVGVPGVGVPGLGVPGVGVP |
| | | GLGVPGVGVPGVGVPGLGVPGVGVPGLGVPGVGVPG |
| 261 | Linker | GYGGGSASG |
| 225 | Fusion | MSKGPGGVGVPGVGVPGVGVPGVGVPGVGVPGVGVPGVGVPGVGVPGVGVPGVGVP |
| | protein | GVGVPGVGVPGVGVPGVGVPGVGVPGVGVPGVGVPGVGVPGVGVPGVGVPGVGVPG |
| | with N- | VGVPGVGVPGVGVPGVGVPGVGVPGVGVPGVGVPGVGVPGVGVPGVGVPGVGVPGV |
| | terminal | GVPGVGVPGVGVPGVGVPGVGVPGVGVPGVGVPGVGVPGVGVPGVGVPGVGVPGVG |
| | metionine | VPGVGVPGVGVPGVGVPGVGVPGVGVPGVGVPGVGVPGVGVPGVGVPGVGVPGVGV |
| | | PGVGVPGVGVPGVGVPGVGVPGVGVPGVGVPGVGVPGVGVPGVGVPGVGVPGVGVP |
| | | GVGVPGVGVPGVGVPGVGVPGVGVPGVGVPGVGVPGVGVPGVGVPGVGVPGVGVPG |
| | | VGVPGVGVPGVGVPGYGGGSASGLEGKVKWFNSEKGFGFIEVEGQDDVFVHFSAIQGE |
| | | GFKTLEEGQAVSFEIVEGNRGPQAANVTKEA |
| 226 | Fusion | SKGPGGVGVPGVGVPGVGVPGVGVPGVGVPGVGVPGVGVPGVGVPGVGVPGVGVPG |
| | protein | VGVPGVGVPGVGVPGVGVPGVGVPGVGVPGVGVPGVGVPGVGVPGVGVPGVGVPGV |
| | without N- | GVPGVGVPGVGVPGVGVPGVGVPGVGVPGVGVPGVGVPGVGVPGVGVPGVGVPGVG |
| | terminal | VPGVGVPGVGVPGVGVPGVGVPGVGVPGVGVPGVGVPGVGVPGVGVPGVGVPGVGV |
| | methionine | PGVGVPGVGVPGVGVPGVGVPGVGVPGVGVPGVGVPGVGVPGVGVPGVGVPGVGVP |
| | | GVGVPGVGVPGVGVPGVGVPGVGVPGVGVPGVGVPGVGVPGVGVPGVGVPGVGVPG |
| | | VGVPGVGVPGVGVPGVGVPGVGVPGVGVPGVGVPGVGVPGVGVPGVGVPGVGVPGV |
| | | GVPGVGVPGVGVPGYGGGSASGLEGKVKWFNSEKGFGFIEVEGQDDVFVHFSAIQGEG |
| | | FKTLEEGQAVSFEIVEGNRGPQAANVTKEA |

### Methods of Using Fusion Proteins

The disclosure provides fusion proteins and methods of using the same. In embodiments, the fusion proteins comprise a nucleic acid binding protein (NBP) which binds to a nucleic acid and a polypeptide with phase behavior. In embodiments, the NBP comprises a Bs-CspB. In embodiments, a composition comprising a nucleic acid is free from one or more contaminants.

In embodiments, a method of purifying a nucleic acid comprises contacting the nucleic acid with a fusion protein; wherein the nucleic acid binds to the fusion protein to form a complex; wherein the size of the complex is increased by a first environmental factor; wherein the complex is separated from at least one contaminant on the basis of size; and wherein the nucleic acid is separated from the fusion protein by a second environmental factor.

In embodiments, a method of purifying a single stranded nucleic acid comprises contacting a composition comprising the single stranded nucleic acid and at least one contaminant with a fusion protein, wherein the fusion protein binds to the single stranded nucleic acid to form a complex, adding to the composition comprising the complex a first environmental factor, thereby increasing the size of the complex, separating the complex from at least one contaminant, and separating the single stranded nucleic acid from the fusion protein by contacting the complex with a second environmental factor, thereby forming a product comprising the single stranded nucleic acid. In embodiments, the single stranded nucleic acid comprises ssRNA, ssDNA or both. In embodiments, the single stranded nucleic acid comprises ssRNA. In embodiments, the at least one contaminant comprises double stranded RNA.

In embodiments, the method comprises separating a complex from at least one contaminant on the basis of size.

In embodiments, a method of purifying a nucleic acid comprises contacting the nucleic acid with a fusion protein; wherein the nucleic acid binds to the fusion protein to form a complex; wherein the size of the complex is increased; wherein the complex is separated from at least one contaminant on the basis of size; and wherein the nucleic acid is separated from the fusion protein by an environmental factor thereby forming a product comprising the nucleic acid. In embodiments, the nucleic acid comprises a single stranded nucleic acid. In embodiments, the nucleic acid comprises ssRNA. In embodiments, the contaminant comprises a double stranded nucleic acid. In embodiments, the contaminant comprises dsRNA.

In embodiments, a method of removing a contaminant from a composition comprising a nucleic acid comprises contacting the contaminant with a fusion protein; wherein the contaminant binds to the fusion protein to form a complex; wherein the size of the complex is increased by a first environmental factor; wherein the complex is separated from the nucleic acid on the basis of size; and wherein the contaminant is separated from the fusion protein by a second environmental factor thereby forming a product comprising the nucleic acid. In embodiments, the nucleic acid comprises a single stranded nucleic acid. In embodiments, the nucleic acid comprises ssRNA. In embodiments, the contaminant comprises a double stranded nucleic acid. In embodiments, the contaminant comprises dsRNA.

In some embodiments, a method of separating a first nucleic acid from a second nucleic acid comprises contacting the first nucleic acid with a first fusion protein and contacting the second nucleic acid with a second fusion protein; wherein the first nucleic acid binds to the first fusion protein to form a first complex; wherein the second nucleic acid binds to the second fusion protein to form a second complex; and separating the first nucleic acid from the second nucleic acid by applying an environmental factor. In embodiments, the first nucleic acid comprises a single stranded nucleic acid. In embodiments, the second nucleic acid comprises a double stranded nucleic acid.

Also provided herein is a method of bringing a nucleic acid in proximity to another nucleic acid. In embodiments, a method of bringing a first nucleic acid into proximity with a second nucleic acid comprises contacting the first nucleic acid with a first fusion protein and contacting the second nucleic acid with a second fusion protein; wherein the first nucleic acid binds to the first fusion protein to form a first complex; wherein the second nucleic acid binds to the second fusion protein to form a second complex; and wherein an environmental factor brings the first complex and second complex into proximity with one another. In embodiments, the methods described herein bring a first nucleic acid and a second nucleic acid within about 10 μm, about 5 μm, about 1 μm, about 900 nm, about 800 nm, about 700 nm, about 600 nm, about 500 nm, about 400 nm, about 300 nm, about 200 nm, about 100 nm, about 10 nm, about 1 nm, about 0.5 nm, or about 0.1 nm of one another. In embodiments, the first nucleic acid comprises a single stranded nucleic acid. In embodiments, the second nucleic acid comprises a double stranded nucleic acid.

In embodiments, the methods described herein utilize a fusion protein comprising a NBP and a polypeptide with phase behavior. In embodiments, the methods described herein utilize a fusion protein comprising a Bs-CspB and a polypeptide with phase behavior. In embodiments, the methods described herein utilize two or more distinct fusion proteins.

In embodiments, the methods described herein involve the formation of a complex. In embodiments, the methods described herein involve the formation of one or more complexes. In embodiments, the methods described herein involve the formation of one, two, three, four, five, or more complexes. The complexes may be referred to as “first complex” or “second complex,” and so on.

In embodiments, a complex comprises a fusion protein and a nucleic acid. In embodiments, a complex comprises a fusion protein and a contaminant. In embodiments, a complex comprises a fusion protein and a second protein such as an enzyme substrate, a metabolite, a ligand (e.g., a ligand that binds to a cellular receptor).

In embodiments, the components of the complex (e.g. the fusion protein and the nucleic acid) bind to each other. In embodiments, the binding is reversible. Reversible binding means that the complexes can dissociate, e.g., separate into individual components. For example, if a complex reversibly forms between the fusion protein and a nucleic acid, the fusion protein and the nucleic acid can subsequently disassociate. In embodiments, dissociation is triggered by an environmental factor. In embodiments, reversible binding allows for separation of a nucleic acid from the fusion protein. In embodiments, reversible binding allows for separation of a contaminant from the fusion protein. In embodiments, reversible binding allows for separation of the other molecule from the fusion protein. In embodiments, the nucleic acid comprises a single stranded nucleic acid. In embodiments, the nucleic acid comprises ssRNA. In embodiments, the contaminant comprises a double stranded nucleic acid. In embodiments, the contaminant comprises dsRNA.

In embodiments, reversible binding is non-covalent, i.e. no covalent bonds are formed between the interacting components of the complex (such as between the fusion protein and the nucleic acid). In embodiments, non-covalent interactions cause the fusion protein and the nucleic acid. Non-limiting examples of non-covalent interactions include dipole-dipole forces, van der Waals forces, London Dispersion forces, hydrogen bonding, hydrophobic interactions, and electrostatic interactions. In embodiments, non-covalent binding is disrupted by the addition of an environmental factor. In embodiments, the nucleic acid comprises a single stranded nucleic acid. In embodiments, the nucleic acid comprises ssRNA.

In embodiments, binding between the fusion protein and a target molecule (e.g., a nucleic acid) is covalent. In embodiments, a covalent bond between a fusion protein and a nucleic acid may be cleaved using, for example, a nuclease and/or a protease. In embodiments, the nucleic acid comprises a single stranded nucleic acid. In embodiments, the nucleic acid comprises ssRNA.

In embodiments, the size of the complexes described herein increase after an environmental factor is applied. In embodiments, the size of a complex formed between the fusion protein and nucleic acid increases. In embodiments, the size of the initial complex increases as a result of aggregation of multiple complexes. In embodiments, multiple complexes aggregate due to self-assembly of fusion proteins. In embodiments, multiple complexes aggregate due to the application of an environmental factor. In embodiments, the size increase is stabilized by non-covalent interactions between multiple fusion proteins. In embodiments, the size increase is stabilized by non-covalent interactions between the polypeptides with phase behavior. In embodiments, the non-covalent interactions are dipole-dipole forces, van der Waals forces, London Dispersion forces, hydrogen bonding, hydrophobic interactions, and/or electrostatic interactions. In embodiments, the nucleic acid comprises a single stranded nucleic acid. In embodiments, the nucleic acid comprises ssRNA.

In embodiments, the methods of the disclosure provide for the formation of multiple complexes in a mixture. In embodiments, the size of all of complexes increase. In embodiments, the size of some complexes increases, and the size of the other complexes remains constant. In embodiments, the size of one complex increases, and the size of the other complex remains constant.

In embodiments, the size of the initial complex increases by at least about 2-fold, at least about 5-fold, at least about 10-fold, at least about 15-fold, at least about 20-fold, at least about 25-fold, at least about 30-fold, at least about 35-fold, at least about 40-fold, at least about 45-fold, at least about 50-fold, at least about 55-fold, at least about 60-fold, at least about 65-fold, at least about 70-fold, at least about 75-fold, at least about 80-fold, at least about 85-fold, at least about 90-fold, at least about 95-fold, at least about 100-fold, or more. In embodiments, the size of the initial complex increases by at least about 2-fold. In embodiments, the size of the initial complex increases by at least about 5-fold. In embodiments, the size of the initial complex increases by at least about 10-fold. In embodiments, the size of the initial complex increases by at least about 25-fold.

As used herein, the phrase “increase in size” may refer to an increase in the diameter of the complex or an increase in the mass of the complex. In embodiments, the increase in size is an increase in the molar mass of the complex. In embodiments, the increase in size is an increase in the hydrodynamic radius of the complex.

In embodiments, the increase in size of the complex can be observed visually with an unaided eye. For example, the increase in size of the complex may cause a composition comprising the complex to change color, clarity, viscosity, and/or may cause the complex to change solubility (e.g., to precipitate from solution), wherein such change is observable by a human without the use of any special equipment.

In embodiments, a person of skill in the art may measure the increase in the size of the complex according to known methods in the art. In embodiments, the increase in the size of the complex can be measured utilizing a technique selected from the group consisting of x-ray scattering, small angle x-ray scattering, wide angle x-ray scattering, dynamic light scattering, analytical ultracentrifugation, size exclusion chromatography, and photon correlation spectroscopy.

In embodiments, the complex of increased size is separated from a contaminant. In embodiments, the complex of increased size containing a nucleic acid and a fusion protein is separated from a contaminant. In embodiments, the complex of increased size containing a contaminant and a fusion protein is separated from a composition containing a nucleic acid. In embodiments, the first complex of increased size containing a first nucleic acid and a first fusion protein is separated from a second complex containing a second nucleic acid and a second fusion protein. In embodiments, the complex of increased size containing a contaminant and a fusion protein is separated from a composition containing a nucleic acid. In embodiments, the first complex of increased size containing a first nucleic acid and a first fusion protein is separated from a second complex containing a second nucleic acid and a second fusion protein. In embodiments, the first nucleic acid comprises a single stranded nucleic acid. In embodiments, the second nucleic acid comprises a single stranded nucleic acid. In embodiments, the single stranded nucleic acid comprises ssRNA. In embodiments, the contaminant comprises a double stranded nucleic acid. In embodiments, the contaminant comprises dsRNA.

In embodiments, separation of the complex from a contaminant can be observed visually with an unaided eye.

In embodiments, separation of the complex from the contaminant is on the basis of size. In embodiments, the separation on the basis of size is performed using a technique selected from the group consisting of tangential flow filtration (TFF), analytical ultracentrifugation, membrane chromatography, high performance liquid chromatography, size exclusion chromatography, membrane chromatography, normal flow filtration, acoustic wave separation, centrifugation, counterflow centrifugation, and fast protein liquid chromatography. In embodiments, the complex is separated from at least one impurity on the basis of size using tangential flow filtration. In embodiments, the complex is separated from at least one impurity on the basis of size using centrifugation. In embodiments, between about 100 relative centrifugal force (RCF) and about 16,000 RCF, for example, about 500 to about 16,000 RCF, about 1,000 RCF to 16,000 RCF, are applied to separate the complex from at least one impurity. In embodiments, at least 500 relative centrifugal force (RCF) are applied to separate the complex from at least one impurity, for example, at least about 500 RCF, at least about 600 RCF, at least about 700 RCF, at least about 800 RCF, at least about 900 RCF, at least about 1000 RCF, at least about 2000 RCF, at least about 3000 RCF, at least about 3500 RCF, at least about 4000 RCF, at least about 5000 RCF, at least about 6000 RCF, at least about 7000 RCF, at least about 8000 RCF, at least about 9000 RCF, at least about 10,000 RCF, at least about 11,000 RCF, at least about 12,000 RCF, at least about 13,000 RCF, at least about 14,000 RCF, at least about 15,000 RCF, at least about 16,000 RCF, at least about 17,000 RCF, at least about 18,000 RCF, at least about 19,000 RCF, or at least about 20,000 RCF.

In embodiments, separation of the complex from the contaminant on the basis of size is performed using TFF. In embodiments, TFF may be used to separate the complex from at least one impurity on the basis of size, a process also referred to herein as “diafiltration.” Diafiltration comprises both washing and elution steps. Washing removes impurities contained in the composition comprising the complexes. Elution separates purified nucleic acids from the fusion protein. In embodiments, the complexes are concentrated using TFF. In embodiments, TFF may be used to increase the concentration of a complex within a composition, a process also referred to herein as “concentration.”

Tangential flow filtration employs both microfiltration and ultrafiltration membranes to separate molecules. Microfiltration membranes typically have pore sizes between 0.1 μm and 10 μm. Ultrafiltration membranes typically have smaller pore sizes than microfiltration membranes with pore sizes between 0.001 μm and 0.1 μm. In embodiments, a membrane with a pore size between about 0.001 μm and about 10 μm is utilized in the methods of the disclosure. In embodiments, the membrane has a pore size of about 0.001 μm, about 0.01 μm, about 0.05 μm, about 0.1 μm, about 0.2 μm, about 0.3 μm, about 0.4 μm, about 0.5 μm, about 0.6 μm, about 0.7 μm, about 0.8 μm, about 0.9 μm, about 1.0 μm, about 2 μm, about 3 μm, about 4 μm, about 5 μm, about 6 μm, about 7 μm, about 8 μm, about 9 μm, or about 10 μm, including all values and ranges in between thereof. In embodiments, the membrane has a pore size of about 0.1 μm. In embodiments, the membrane has a pore size of about 0.2 μm.

In embodiments, the membrane is made of hydrophilized poly(vinylildene difluoride) (PVDF), polyetheresulfone (PES), cellulose phosphate, diethylaminoethyl cellulose, polysufone, regenerated cellulose, nylon, cellulose nitrate, cellulose acetate, pegylated PES, modified polyethersulfone, and sulfonated PES, or modified derivatives thereof of the aforementioned materials.

In TFF, a membrane is placed tangentially to the flow of a fluid mixture to cause the fluid mixture to flow tangentially over a first side of the membrane. At the same time, a fluid media is placed in contact with a second surface of the membrane. A transmembrane pressure is the force that drives fluid through the membrane, carrying along permeable molecules.

In embodiments, separation of the complex from the contaminant on the basis of size is performed using TFF with a transmembrane pressure of between about 0.1 bar to about 3 bar. In embodiments, the transmembrane pressure is about 0.1 bar, about 0.2 bar, about 0.3 bar, about 0.4 bar, about 0.5 bar, about 0.6 bar, about 0.7 bar, about 0.8 bar, about 0.9 bar, about 1.0 bar, about 1.1 bar, about 1.2 bar, about 1.3 bar, about 1.4 bar, about 1.5 bar, about 1.6 bar, about 1.7 bar, about 1.8 bar, about 1.9 bar, about 2.0 bar, about 2.1 bar, about 2.2 bar, about 2.3 bar, about 2.4 bar, about 2.5 bar, about 2.6 bar, about 2.7 bar, about 2.8 bar, about 2.9 bar, or about 3.0 bar, including all values and ranges in between. In embodiments, the transmembrane pressure is about 1.5 bar.

In embodiments, the cross flow rate is tuned to improve the separation of the complexes described herein from the contaminant. The cross flow rate is the rate of solution flow through the feed channel and across the membrane. It provides the force that sweeps away molecules that can restrict filtrate flow. In embodiments, the cross flow rate is between about 500 L/m²/h and about 2000 L/m²/h. In embodiments, the cross flow rate is between about 500 L/m²/h, about 600 L/m²/h, about 700 L/m²/h, about 800 L/m²/h, about 900 L/m²/h, about 1000 L/m²/h, about 1100 L/m²/h, about 1200 L/m²/h, about 1300 L/m²/h, about 1400 L/m²/h, about 1500 L/m²/h, about 1600 L/m²/h, about 1700 L/m²/h, about 1800 L/m²/h, about 1900 L/m²/h, or about 2000 L/m²/h, including all values and ranges in between thereof. In embodiments, the cross flow rate is about 960 L/m²/h. In embodiments, TFF separation occurs by using a membrane that retains the complex containing the fusion protein and the nucleic while passing the contaminant. In embodiments, a membrane that retains the complex containing the fusion protein and the contaminant while passing the nucleic acid is used. In embodiments, a membrane that retains the complex containing the fusion protein and the contaminant while passing the nucleic acid is utilized. In embodiments, a membrane that retains the first complex containing the fusion protein and the first nucleic acid while passing the complex containing the second fusion protein and second nucleic acid is utilized. In embodiments, the first and second nucleic acid comprise a single stranded nucleic acid. In embodiments, the first and second nucleic acid comprise ssRNA. In embodiments, the contaminant comprises a double stranded nucleic acid. In embodiments, the contaminant comprises dsRNA.

In embodiments, the methods described herein enable the purification of at least 0.1 kg, at least about 0.2 kg, at least about 0.3 kg, at least about 0.4 kg, at least about 0.5 kg, at least about 0.6 kg, at least about 0.7 kg, at least about 0.8 kg, at least about 0.9 kg, at least about 1 kg, at least about 2 kg, at least about 3 kg, at least about 4 kg, at least about 5 kg, at least about 6 kg, at least about 7 kg, at least about 8 kg, at least about 9 kg, at least about 10 kg, or more of nucleic acid per day, including all values and ranges in between.

In embodiments, the methods described herein are completed in about 0.5 hr to about 24 hours. In embodiments, the methods are completed in about 0.5 hr, about 1 hr, about 2 hr, about 3 hr, about 4 hr, about 5 hr, about 6 hr, about 7 hr, about 8 hr, about 9 hr, about 10 hr, about 11 hr, about 12 hr, about 13 hr, about 14 hr, about 15 hr, about 16 hr, about 17 hr, about 18 hr, about 19 hr, about 20 hr, about 21 hr, about 22 hr, about 23 hr, or about 24 hr. In embodiments, the methods described herein are completed in about 0.5 hr to about 8 hr. In embodiments, the methods of the disclosure are completed in about 2 hr to about 6 hr.

In embodiments, the methods described herein produce a final product comprising a proportion of a target nucleic acid based on a total nucleic acid content. In embodiments, the proportion of the target nucleic acid based on the total nucleic acid content can be calculated as a purification yield. In embodiments, the nucleic acid is a single stranded nucleic acid.

In embodiments, the purification yield of the nucleic acid is at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In embodiments, the nucleic acid is purified to at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In embodiments, the purification yield of the single stranded nucleic acid is at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% purity.

In embodiments, the single stranded nucleic acid is purified to at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In embodiments, the purification yield of the ssRNA is at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In embodiments, the ssRNA is purified to at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In embodiments, the purified nucleic acid retains its biological activity and/or structure. In embodiments, the purified nucleic acid has enhanced biological activity. In embodiments, the purified single stranded nucleic acid retains its biological activity and/or structure. In embodiments, the purified single stranded nucleic acid has enhanced biological activity. In embodiments, the ssRNA retains its biological activity and/or structure. In embodiments, the purified ssRNA has enhanced biological activity.

In embodiments, at least 400 g/m² (grams of nucleic acid per m² of filter membrane) are purified per day. In embodiments, at least 400 g/m², at least 500 g/m², at least 600 g/m², at least 700 g/m², at least 800 g/m², at least 900 g/m², or at least 1000 g/m² of nucleic are purified per day. In embodiments, the nucleic acid is e.g., mRNA, ssRNA or a virus. In embodiments, the nucleic acid is ssRNA.

In embodiments, at least about 150 g/L (grams of nucleic per liters of fusion protein) are purified per day. In embodiments, at least about 150 g/L, at least about 200 g/L, at least about 250 g/L, at least about 300 g/L, at least about 350 g/L, at least about 400 g/L, at least about 450 g/L, at least about 500 g/L, at least about 550 g/L, at least about 600 g/L, at least about 650 g/L, at least about 700 g/L, at least about 750 g/L, at least about 800 g/L, at least about 850 g/L, at least about 900 g/L, at least about 950 g/L, or at least about 1000 g/L are purified per day. In embodiments, the nucleic acid is e.g., mRNA, ssRNA, or a virus. In embodiments, the nucleic acid is ssRNA.

In embodiments, the product comprises about 70% to about 100% of the single stranded nucleic acid. In embodiments, the product comprises at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the single nucleic acid. In embodiments, the single stranded nucleic acid comprises ssRNA.

In embodiments, a final product comprises about 10% or less of the at least one contaminant. In embodiments, the product comprises about 0.1%, about 1%, about 2%, about 3%, about 4%, about 5%, about 6%, about 7%, about 8%, about 9%, or about 10% of the at least one contaminant. In the embodiments, the purification method comprises at least about a 3-log removal of a contaminant in the final product compared to an amount of a contaminant in an original composition. In the embodiments, the purification method comprises least about a 1-log, 2-log, 3-log, 4-log, 5-log, 6-log, 7-log, 8-log, 9-log or 10-log removal of a contaminant compared to an amount of a contaminant in an original composition. In the embodiments, the purification method comprises at least about a 3-log removal to 10-log removal of a contaminant compared to an amount of a contaminant in an original composition. In embodiments, the contaminant comprises dsRNA.

In embodiments, the fusion protein is present at a concentration of about 1 μM to about 200 μM (e.g., 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μM, including all ranges and values therein). In embodiments, the fusion protein is present at a concentration of about 30 μM to about 50 μM. In embodiments, the fusion protein is present at a concentration of about 40 μM.

### Environmental Factors

In embodiments, one or more environmental factors are applied to cause a change of a complex comprising the fusion protein and nucleic acid. In embodiments, the one or more environmental factors cause the size of a complex comprising the fusion protein and nucleic acid to increase. In embodiments, the one or more environmental factors cause the polypeptide with phase behavior to aggregate. In embodiments, the one or more environmental factors causes separation of the fusion protein from the nucleic acid. In embodiments, the one or more environmental factors causes separation of the fusion protein from the contaminant. In embodiments, the one or more environmental factors enables the nucleic acid to retain its native structure, function, and activity.

In embodiments, the environmental factor is a change in temperature. In embodiments, the temperature is increased about 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In embodiments, the temperature is decreased about 0.5° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In embodiments, the environmental factor is a change in pH. In embodiments, the pH is increased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units. In embodiments, the pH is decreased by about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0 units.

In embodiments, the environmental factor is change in ionic strength. In embodiments, the change in ionic strength is brought about by increasing the concentration of salt. In embodiments, the change in ionic strength is brought about by decreasing the concentration of salt. Non-limiting examples of salts include sodium chloride, potassium chloride, ammonium chloride, sodium acetate, sodium citrate, copper sulfate, sodium iodide, ammonium sulfate, and sodium sulfate. In embodiments, dialysis is used to change the concentration of salt in the composition containing the fusion protein and nucleic acid and/or contaminant. In embodiments, the salt is added at a concentration comprising about 0.5 M to about 3 M (e.g., 0.5, 1, 1.5, 2, 2.5, or 3 M, including any ranges or values therein).

In embodiments, the environmental factor is the addition of a cofactor. Non-limiting examples of cofactors include calcium, magnesium, cobalt, copper, zinc, iron, manganese, selenium, molybdenum, potassium, coenzyme A (CoA), a nucleoside triphosphate, and a vitamin. In embodiments, the cofactor is calcium. In embodiments, the nucleoside triphosphate is adenosine triphosphate, uridine triphosphate, guanosine triphosphate, cytidine triphosphate, or thymidine triphosphate. In embodiments, the vitamin is fat-soluble. In embodiments, the vitamin is water-soluble. Non-limiting examples of vitamins include vitamin A, vitamin B1 (thiamine), vitamin B2 (riboflavin), vitamin B3 (niacin or niacinamide), vitamin B5 (pantothenic acid), Vitamin B6 (pyridoxine, pyridoxal, or pyridoxamine, or pyridoxine hydrochloride), vitamin B7 (biotin), vitamin B9 (folic acid), vitamin B12, vitamin C, vitamin D, Vitamin E, vitamin K, K1, and K2, folic acid, and biotin.

In embodiments, the environmental factor is a change in the concentration of the fusion protein. In embodiments, the environmental factor is a change in the concentration of the nucleic acid. In embodiments, the environmental factor is a change in the concentration of the contaminant.

In embodiments, the environmental factor is a change in pressure of the composition containing the fusion protein and nucleic acid. In embodiments, the environmental factor is a change in pressure of the composition containing the fusion protein and contaminant. In embodiments, a change in pressure can be effected by increasing or decreasing the volume of the composition.

In embodiments, the environmental factor is the addition of one or more surfactants. In embodiments, the one or more surfactants are selected from free fatty acid salts, soaps, fatty acid sulfonates, such as sodium lauryl sulfate, ethoxylated compounds, such as ethoxylated propylene glycol, lecithin, polygluconates, quaternary ammonium salts, lignin sulfonates, 3-((3-cholamidopropyl) dimethylammonio)-1-propanesulfonate (CHAPS), sugars, including sucrose and glucose, Triton X-100, and NP-40. In embodiments, the surfactant is anionic, nonionic, or amphoteric.

In embodiments, the environmental factor is the addition of one or more molecular crowding agents. Non-limiting examples of molecular crowding agents include polyethylene glycol, dextran, and ficoll. Non-limiting examples of PEGS include PEG400, PEG1450, PEG3000, PEG8000, and PEG10000.

In embodiments, the environmental factor is the addition of one or more oxidizing agents. Non-limiting examples of oxidizing agents include hydrogen peroxide, hydrophilically or hydrophobically activated hydrogen peroxide, preformed peracids, monopersulfate or hypochlorite.

In embodiments, the environmental factor is the addition of one or more reducing agents. In embodiments, the one or more reducing agents is selected from the group consisting of dithiothreitol (DTT), 2-mercaptoethanol (BME), Tris (2-carboxyethyl) phosphine (TCEP), hydrazine, boron hydrides, amine boranes, lower alkyl substituted amine boranes, triethanolamine, and N,N,N′,N′-tetramethylethylenediamine (TEMED).

In embodiments, the environmental factor is the addition of one or more denaturing agents. Non-limiting examples of denaturing agents include urea, guanidine hydrochloride, guanidine, sodium salicylate, dimethyl sulfoxide, and propylene glycol.

In embodiments, the environmental factor is the addition of one or more enzymes. Non-limiting examples of enzymes include proteases, kinases, phosphatases, synthetases, transferases, nucleases such as restriction endonucleases, lyases, isomerases, dehydrogenases, decarboxylases, and lipases.

In embodiments, the environmental factor is the application of electromagnetic waves. In embodiments, the environmental factor is the application of light. In embodiments, the electromagnetic waves have a wavelength between about 0.0001 nm and about 100 m. In embodiments, the electromagnetic waves are selected from the group consisting of gamma rays, x-rays, ultraviolet, visible, infrared, and radio waves. In embodiments, the electromagnetic waves are gamma rays. In embodiments, the gamma rays have a wavelength between about 0.0001 nm and about 0.01 nm, e.g. 0.0001 nm, 0.0005 nm, 0.001 nm, 0.002 nm, 0.003 nm, 0.004 nm, 0.005 nm, 0.006 nm, 0.007 nm, 0.008 nm, 0.009 nm, and 0.01 nm. In embodiments, the x-rays have a wavelength between about 0.01 nm and 10 nm, e.g. about 0.01 nm, 0.02 nm, 0.03 nm, 0.04 nm, 0.05 nm, 0.06 nm, 0.07 nm, 0.08 nm, 0.09 nm, 0.10 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or about 10 nm. In embodiments, the ultraviolet radiation has a wavelength between about 10 nm about 400 nm, e.g. about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 280 nm, about 300 nm, about 350 nm, or about 400 nm. In embodiments, the visible waves have a wavelength of between about 400 nm and about 800 nm, e.g. about 400 nm, about 450 nm, about 500 nm, about 550 nm, about 600 nm, about 650 nm, about 700 nm, about 750 nm, or about 800 nm. In embodiments, the infrared radiation has a wavelength of between about 800 nm and about 0.1 cm, e.g. about 800 nm, about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 0.1 cm. In embodiments, the radio waves has a wavelength of between about 0.1 cm and 100 m, e.g. about 0.1 cm, about 1 cm, about 10 cm, about 100 cm, about 1000 cm, about 2000 cm, about 3000 cm, about 4000 cm, about 5000 cm, about 6000 cm, about 7000 cm, about 8000 cm, about 9000 cm, or about 100 m.

In embodiments, the environmental factor is the application of acoustic waves. In embodiments, the acoustic waves have a frequency between about 1 Hz and 2000 kHz. In embodiments, the acoustic waves have a frequency of about 1 Hz, about 5 Hz, about 10 Hz, about 20 Hz, about 30 Hz, about 40 Hz, about 50 Hz, about 60 Hz, about 70 Hz, about 80 Hz, about 90 Hz, about 100 Hz, about 200 Hz, about 300 Hz, about 400 Hz, about 500 Hz, about 600 Hz, about 700 Hz, about 800 Hz, about 900 Hz, about 1 kHz, about 100 kHz, about 200 kHz, about 300 kHz, about 400 kHz, about 500 kHz, about 600 kHz, about 700 kHz, about 800 kHz, about 900 kHz, about 1000 kHz, about 1100 kHz, about 1200 kHz, about 1300 kHz, about 1400 kHz, about 1500 kHz, about 1600 kHz, about 1700 kHz, about 1800 kHz, about 1900 kHz, or about 2000 kHz.

### NUMBERED EMBODIMENTS

In addition to the appended claims, the following numbered embodiments also form part of the instant disclosure.

1. A fusion protein comprising a nucleic acid binding protein (NBP) and a polypeptide with phase behavior.

2. The fusion protein of claim 1, wherein the NBP is selected from any one of: RNA-specific adenosine deaminase 1 (ADAR1), ADAR1 double stranded RNA-binding domain 3 (dsRBD3), Bacillus subtilis cold shock protein B (Bs-CspB), cold shock domain Y-box protein (CSD-Ybox), eukaryotic translation initiation factor 4E (eIF4e), Fox-1 protein (FOX1), heterogenous nuclear ribonucleoprotein Q1 (hnRNPQ1), Homo sapiens zinc finger CCCH-Type containing 14 (HsZC3H14), polyA-binding protein (PABP), polyA-binding protein nuclear 1 (PABPN1), pentatricopeptide repeat protein A (PPRpA), Pumilio-like repeat protein A (PUFpA), Staufen, 12-O-tetradecanoylphorbol-13-acetate inducible sequence 11 D (TIS11D), Z-DNA/RNA binding protein 1 (ZBP1), and Zinc Finger Nuclease (ZNF).

3. The fusion protein of claim 1, wherein the NBP has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 61-103.

4. The fusion protein of any one of claims 1-3, wherein the NBP is selected from any one of: ADAR1 double stranded RNA-binding domain 3 (dsRBD3), heterogenous nuclear ribonucleoprotein Q1 (hnRNPQ1), Homo sapiens zinc finger CCCH-Type containing 14 (HsZC3H14), polyA-binding protein nuclear 1 (PABPN1), pentatricopeptide repeat protein A (PPRpA), and Pumilio-like repeat protein A (PUFpA).

5. The fusion protein of any one of claims 1-4, wherein the NBP has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99 or 100% identity to a polypeptide of any one of SEQ ID NOS: 89, 93, 95, or 97-99.

6. The fusion protein of any one of claims 1-5, wherein the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-60 or 217.

7. The fusion protein of any one of claims 1-6, wherein the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 57 or 60.

8. The fusion protein of any one of claims 1-7, comprising a linker.

9. The fusion protein of claim 8, wherein the linker has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 143-216.

10. The fusion protein of any one of claims 1-9, wherein the NBP binds to RNA, DNA, or both.

11. The fusion protein of any one of claims 1-10, wherein the NBP binds to RNA selected from any one of double stranded RNA (dsRNA), single stranded RNA (ssRNA), mRNA, pre-mRNA, polyadenosine (polyA) RNA, Z-confirmation RNA (Z-RNA), or a combination thereof.

12. The fusion protein of any one of claims 1-11, wherein the NBP binds to the 3′-terminus of mRNA, the 3′-terminus of mRNA, a 3′ untranslated region (UTR) of mRNA, a polyA tail of mRNA, or an AU-rich element, or combination thereof, of mRNA.

13. The fusion protein of any one of claims 1-11, wherein the NBP binds to a pre-mRNA.

14. The fusion protein of claim 13, wherein the NBP binds to an intron, exon, polyA tail, or combination thereof, of the pre-mRNA.

15. The fusion protein of any one of claims 1-10, wherein the NBP binds to DNA.

16. The fusion protein of claim 15, wherein the NBP binds to single stranded DNA, double stranded DNA, polyadenosine (polyA) DNA, Z-conformation DNA (Z-DNA), or a combination thereof.

17. A nucleic acid encoding the fusion protein of any one of claims 1-16.

18. The nucleic acid of claim 17 having a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 119-133.

19. A vector encoding a fusion protein of any one of claims 1-16.

20. A vector comprising the nucleic acid of any one of claims 17-18.

21. A vector having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a nucleic acid of any one of SEQ ID NOS: 104-118, 134, and 135.

22. A method of purifying a nucleic acid comprising: (i) contacting a composition comprising the nucleic acid and at least one contaminant with a fusion protein of any one of claims 1-16, wherein the fusion protein binds to the nucleic acid to form a complex; (ii) contacting the complex with a first environmental factor to increase the size of the complex; (iii) separating the complex from at least one contaminant; and (iv) separating the nucleic acid from the fusion protein by contacting the complex with a second environmental factor.

23. The method of claim 22, wherein the complex is separated from the at least one contaminant on the basis of size.

24. The method of claim 23, wherein the separation on the basis of size is performed using a method selected from any one of tangential flow filtration, membrane chromatography, analytical ultracentrifugation, high performance liquid chromatography, membrane chromatography, normal flow filtration, acoustic wave separation, centrifugation, counterflow centrifugation, and fast protein liquid chromatography.

25. The method of any one of claims 22-24, wherein the first environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves.

26. The method of any one of claims 22-25, wherein the second environmental factor comprises one or more of: (a) a change in one or more of temperature, pH, salt concentration, concentration of the purification matrix, concentration of the viral particle, or pressure; (b) the addition of one or more surfactants, cofactor, vitamin, molecular crowding agents, reducing agents, oxidizing agents, enzymes, or denaturing agents; or (c) the application of electromagnetic or acoustic waves.

27. The method of any one of claims 22-26, wherein the at least one contaminant is selected from a solvent, a protein, a peptide, a carbohydrate, a nucleic acid, a virus, a cell (e.g., a bacterial, yeast, or mammalian cell), a carbohydrate, a lipid, or a lipopolysaccharide.

### EXAMPLES

### Example 1. Design of Fusion Proteins Containing Nucleic Acid Binding Proteins and Polypeptides with Phase Behavior

A fusion protein comprising an NBP and a polypeptide with phase behavior is generated and characterized. The NBP comprises a sequence with at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 61-103. The polypeptide with phase behavior comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1-60 or 217. The fusion protein comprising the NBP and polypeptide with phase behavior is expressed according to standard protocols. Affinity of the fusion protein for a nucleic acid is assessed through capture experiments. The transition temperature of the fusion protein is determined using UV-Vis spectrophotometry.

### Example 2. Purification of Nucleic Acid Using Fusion Protein of Example 1

The fusion proteins of Example 1 are used to purify a target nucleic acid. The fusion protein is mixed with a sample containing the target nucleic acid and a contaminant. The fusion protein binds to the target nucleic acid. A first environmental factor is added to the composition to increase the size of the complex. TFF is used to separate the complex from the contaminants. A second environmental factor is added to a solution containing the purified complex to separate the fusion protein from the nucleic acid.

### Example 3. Comparison of ITC Generated Reagents Comprising Different Polypeptides with Phase Behavior for Non-Specific Binding

Purpose: This objective of this study was to identify the most effective polypeptide with phase behavior, for use in purifying single stranded nucleic acids.

Methods: This study evaluated the non-specific binding capabilities of different polypeptides with phase behavior alone to a variety of nucleic acids. Affinity of the polypeptide with phase behavior for a nucleic acid was assessed as described in Example 2. Nucleic acids were synthesized, mixed with different polypeptides with phase behavior, and then purified as described in Example 2 (e.g., using inverse transition cycling). The final concentrations of polypeptide with phase behavior, nucleic acid and salt within the capture reaction were 40 μM, 0.1 mg/ml, and 1.5M, respectively. Subsequently, 10 μl of capture reaction were loaded into the wells for the study. RNA templates of varying length and strandedness, in addition to linearized plasmid DNA, were tested for binding against four different polypeptides with phase behavior (20A80 (SEQ ID NO: 262), 50A80 (SEQ ID NO: 263), 100V80 (SEQ ID NO: 56), and 40L80 (SEQ ID NO: 264)). Capture reactions were loaded directly onto 1% native agarose gels such that successful binding of the nucleic acid species by the polypeptides with phase behavior would be indicated as retention within the well.

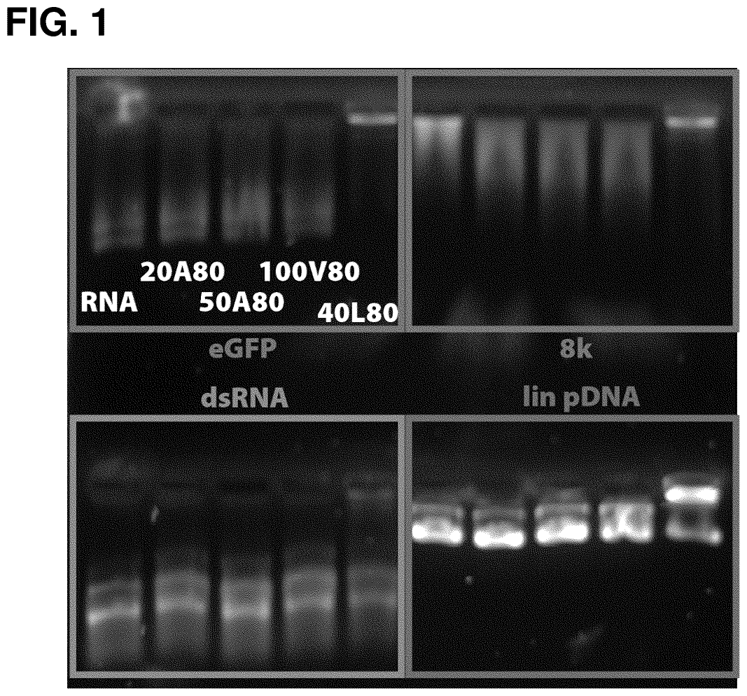

Results: Results are in FIG. 1. As shown, 20A80, 50A80 and 100V80 had little-to-no non-specific binding, while 40L80 exhibited non-specific binding to a broad range of nucleic acid species and was thus less effective for nucleic acid capture. Among the polypeptides with phase behavior, 100V80 also demonstrated the most optimal working temperatures for inverse transition cycling during production. Therefore, 100V80 was determined to be the most successful candidate polypeptide with phase behavior for the development of a fusion protein.

### Example 4. Effective Elution of Single Stranded RNA During an In Vitro Transcription Reaction Using a Reagent Comprising Bs-CspB and a Polypeptide with Phase Behavior

Purpose: This study sought to evaluate the purification of single stranded RNA (ssRNA) during in vitro transcription using a reagent comprising a fusion protein (SEQ ID NO: 225) containing the NBP Bs-CspB (SEQ ID NO: 90) and a polypeptide with phase behavior referred to as 100V80 (SEQ ID NO: 56).

Method: This experiment was performed using a shake-flask generated and inverse transition cycling (ITC) purified ssRNA purification reagent (referred to as “Isotag”). RNA capture reactions were then performed using the ssRNA purification reagent from in vitro transcription (IVT) reactions separately transcribing a range of mRNA template sizes including 1 kb, 4 kb, and 8 kb.

Direct IVT captures were performed using the ITC generated ssRNA purification reagent. To each IVT reaction, 50 μM of reagent was added to capture the desired ssRNA present in the reaction. The addition of NaCl to achieve a 1.5M final concentration permitted the phase separation necessary for size-based separation of the captured RNA within droplets from other IVT impurities using centrifugation at room temperature (RT). Following aspiration of the capture supernatant, water was used to disassociate the RNA from the droplets which were kept intact in the presence of heat. Another round of centrifugation separated the droplets from the final purified RNA. To determine nucleic acid concentration within the IVT reaction, lithium acetate precipitation and subsequent ethanol washes were used to precipitate and purify the RNA present in IVT reaction prior to resuspension in water. RNA concentration was calculated by assessing absorbance at 260 nm and 280 nm with a UV spectrometer and calculating concentration using the Beer-Lambert law. The percent elution of total RNA was then calculated for each mRNA template, including 1 kb, 4 kb and 8 kb. The percent elution reflected the final yield post-elution compared to the total RNA present in the initial IVT reaction.

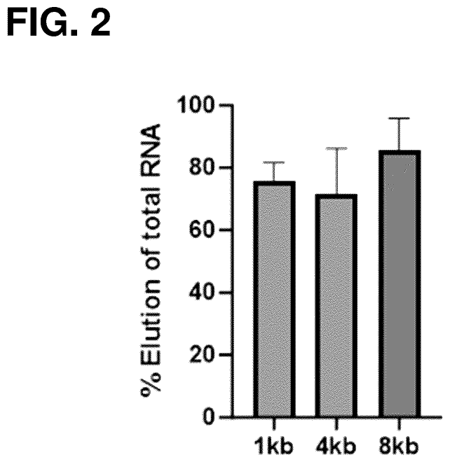

Results: Results are in FIG. 2. In sum, the ssRNA purification reagent was able to obtain over 80% recovery of ssRNA during ITC reactions. These findings demonstrate the successful use of the fusion protein comprising Bs-CspB and the polypeptide with phase behavior 100V80 to effectively purify target ssRNA using size-based separation.

### Example 5. Highly Selective Capture of Purified RNA Using the ITC Generated Reagent of Example 4

Purpose: This study sought to evaluate the specificity by which the ITC generated reagent of Example 4 selectively captures target ssRNA by quantifying the separation of contaminants, exemplified by dsRNA.

Method: This study examined the capability of the ssRNA purification reagent of Example 3 to selectively capture purified RNA. For this study, separation of dsRNA from ssRNA was evaluated across a range of ssRNA purification reagent:RNA molar ratios.

To determine the Log 10 removal value (LRV) of dsRNA, the dsRNA concentrations in solution were assessed before and after purification with the ITC generated reagent. The RNA was generated with the HiScribe® T7 Quick High Yield RNA Synthesis Kit (NEB) and a linearized eGFP template bearing 5′ and 3′ UTRs with a 120-nucleotide poly A tail. IVT was carried out at 20° C. O/N to ensure sufficient generation of dsRNA. To test specificity for ssRNA with elevating impurities of dsRNA, ssRNA was generated via IVT utilizing the Hi-T7® RNA Polymerase (NEB) and subsequently enriched for ssRNA using cellulose based ssRNA purification while dsRNA matching the sequence and size of the ssRNA was also generated and purified as described in Baiersdorfer et al, Mol. Ther Nucleic Acids, 2019. UV-spectrometry was used to assess ssRNA concentrations while dsRNA concentrations were tested using a multi-species dsRNA ELISA kit (Novus Biologicals). Moreover, the percent elution of total dsRNA was evaluated among total RNA containing a range of percent spiked dsRNA including 0%, 0.01%, 0.1%, 1% and 10%.

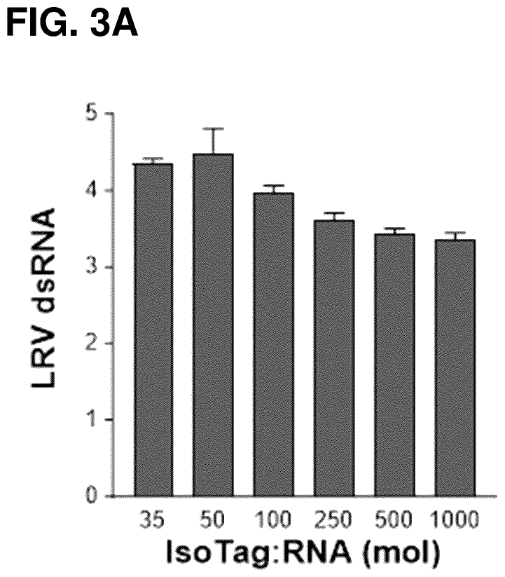

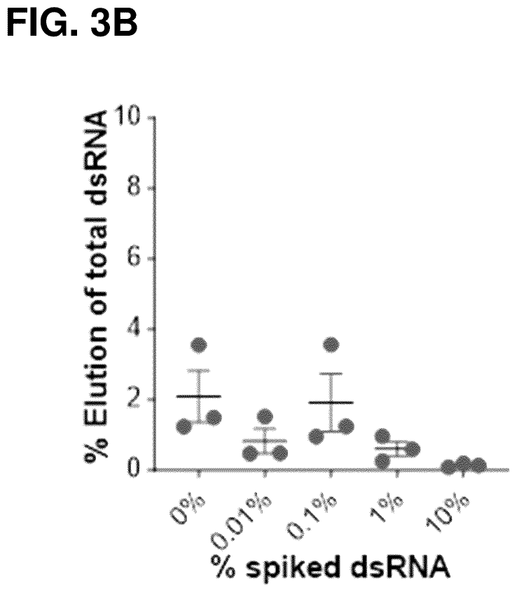

Results: Results are shown in FIGS. 3A and 3B. In sum, the ssRNA purification reagent was highly selective for ssRNA. As shown in FIG. 3A, the resulting RNA captures of purified RNA with spiked in dsRNA demonstrated that across a range of ssRNA purification reagent:RNA molar ratios, the reagent retained selectivity for ssRNA and was capable of greater than 4-log removal of dsDNA. Moreover, FIG. 3B shows that despite spiking in dsRNA at values as high as 10% of the total RNA, selectivity was retained in captures performed on purified RNA. Regardless of the dsRNA content added, the percent eluted during the purification process remained less than 4%. These findings indicate that the reagent comprising Bs-CspB and 100V80 is both highly selective for the target nucleic acid and is able to separate the majority of contaminants from the final product.

### Example 6. Comparison of the Bs-CspB and 100V80 Reagent Versus Other Nucleic Binding Protein Reagents for Binding ssRNA and Other Sequences

Purpose: This study sought to evaluate the ability of the ITC generated reagent described in examples 4 and 5, comprising Bs-CspB and 100V80, to bind to a broad range of ssRNA templates and other sequences, and compare that ability to other ITC generated reagents comprising different nucleic binding proteins with 100V80.

Method: This study assessed nucleic acid capture of purified RNA using several different ITC generated reagents. A HiScribe T7 ARCA mRNA kit (NEB) was used to synthesize RNA from a PCR generated DNA template or linearized plasmid followed by post-IVT tailing for transcripts bearing a poly A tail. A 513 bp dsRNA was generated using a luciferase template following the method as described in Baiersdorfer et al, Mol Ther Nucleic Acids, 2019. RNA was purified by phenol-chloroform extraction followed by ethanol precipitation with ammonium acetate. The final RNA pellet was resuspended at 1 mg/ml in ultrapure water. UV-Vis spectrometry and gel electrophoresis were used to confirm purity and length of the mRNA transcript. The final concentrations of biopolymer, RNA and salt within the capture reaction were 40 μM, 0.1 mg/ml, and 1.5M, respectively. Capture supernatant was aspirated following centrifugation for 5 min at 5000 g. Successful capture was assessed by gel electrophoresis of samples loaded separately onto a 1% native agarose gel supplemented with 1× Sybr Safe DNA Gel Stain.

The ssRNA binding candidates, consisting of various nucleic binding proteins coupled to 100V80, were screened for their ability to capture ssRNA and dsRNA targets in independent capture reactions. Agarose gel electrophoresis was used to test for the presence of nucleic acid species in the capture supernatant. The presence of a band on the gels indicates uncaptured nucleic acids.

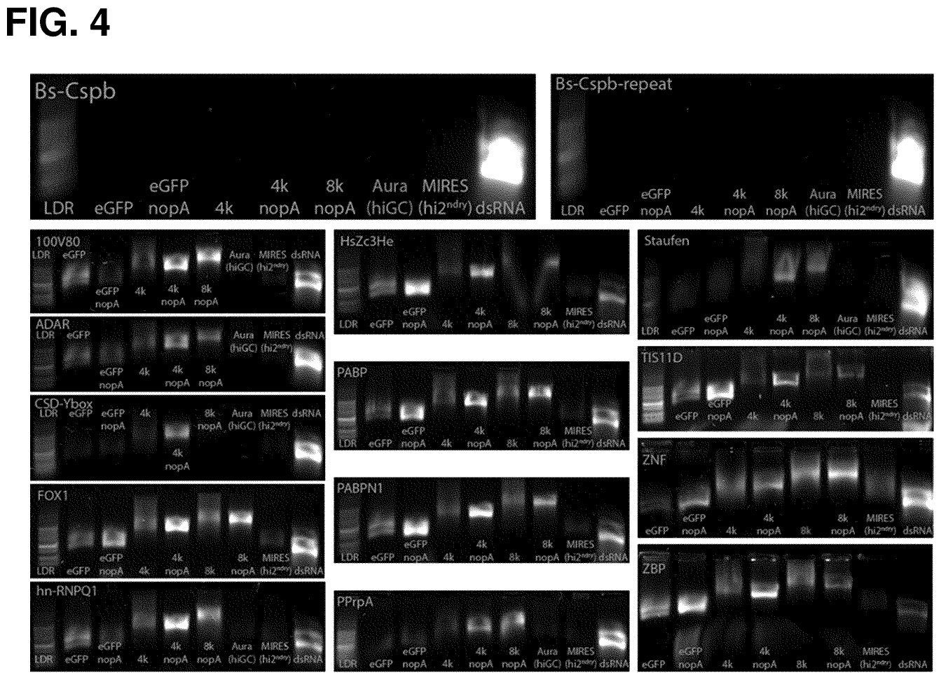

Results: Results are in FIG. 4. As shown, the Bs-CspB and 100V80 reagent is absent of any band for the ssRNA templates, and demonstrates a large amount of dsRNA that remained uncaptured. Overall, the Bs-CspB reagent demonstrates remarkable capture of a broad range of ssRNA species while not retaining dsRNA within the droplets. In sum, these findings demonstrate the successful use of a fusion protein comprising Bs-CspB and 100V80 to purify single stranded nucleic acids.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that it constitutes valid prior art or form part of the common general knowledge in any country in the world. The following patent documents are incorporated by reference herein in their entireties for all purposes: International Patent Publication No. 2021/168270 and International Patent Publication No. 2022/178537.

1. A fusion protein comprising a Bacillus subtilis cold shock protein B (Bs-CspB) and a polypeptide with phase behavior.

2. The fusion protein of claim 1, wherein the Bs-CspB has an amino acid sequence with at least 95% identity to a polypeptide of SEQ ID NO: 90.

3. The fusion protein of claim 1, wherein the polypeptide with phase behavior comprises a P and G motif, comprising a plurality of proline residues and a plurality of glycine residues.

4. The fusion protein of claim 1, wherein the P and G motif comprises at least about 10% proline residues and at least about 20% glycine residues.

5. The fusion protein of claim 1, wherein the polypeptide with phase behavior comprises pentapeptide repeat having the sequence (Val-Pro-Gly-Xaa-Gly)n (SEQ ID NO: 217), or a randomized, scrambled analog thereof; wherein Xaa can be any amino acid except proline, wherein n is an integer from 1 to 360, inclusive of endpoints.

6. (canceled)

7. The fusion protein of claim 1, wherein the polypeptide with phase behavior comprises an amino acid sequence selected from:
a.
(SEQ ID NO: 1)
(GRGDSPY)ₙ;
b.
(SEQ ID NO: 2)
(GRGDSPH)ₙ;
c.
(SEQ ID NO: 3)
(GRGDSPV)ₙ;
d.
(SEQ ID NO: 4)
(GRGDSPYG)ₙ;
e.
(SEQ ID NO: 5)
(RPLGYDS)ₙ;
f.
(SEQ ID NO: 6)
(RPAGYDS)ₙ;
g.
(SEQ ID NO: 7)
(GRGDSYP)ₙ;
h.
(SEQ ID NO: 8)
(GRGDSPYQ)ₙ;
i.
(SEQ ID NO: 9)
(GRGNSPYG)ₙ;
j.
(SEQ ID NO: 10)
(GVGVP)ₙ;;
k.
(SEQ ID NO: 11)
(GVGVPGLGVPGVGVPGLGVPGVGVP)ₘ;
l.
(SEQ ID NO: 12)
(GVGVPGVGVPGAGVPGVGVPGVGVP)ₘ;
m.
(SEQ ID NO: 13)
(GVGVPGWGVPGVGVPGWGVPGVGVP)ₘ;
n.
(SEQ ID NO: 14)
(GVGVPGVGVPGVGVPGVGVPGVGVPGVGVPGEGVPGFGVPGVGVP)ₘ;
o.
(SEQ ID NO: 15)
(GVGVPGVGVPGVGVPGVGVPGVGVPGVGVPGKGVPGFGVPGVGVP)ₘ;
and
p.
(SEQ ID NO: 16)
(GAGVPGVGVPGAGVPGVGVPGAGVP)ₘ;
wherein:
n is an integer in the range of 20-360, inclusive of endpoints; and
m is an integer in the range of 4-25, inclusive of endpoints.

8. The fusion protein of claim 1, wherein the polypeptide with phase behavior comprises an amino acid sequence selected from:
a.
(SEQ ID NO: 22)
(GVGVP)ₘ;
b.
(SEQ ID NO: 23)
(ZZPXXXXGZ)ₘ;
c.
(SEQ ID NO: 24)
(ZZPXGZ)ₘ;
d.
(SEQ ID NO: 25)
(ZZPXXGZ)ₘ;
or
e.
(SEQ ID NO: 26)
(ZZPXXXGZ)ₘ,
wherein m is an integer between 10 and 160, inclusive of endpoints, wherein X if present is any amino acid except proline or glycine, and wherein Z if present is any amino acid.

9. The fusion protein of claim 1, wherein the polypeptide with
phase behavior comprises an amino acid sequence selected from:
a.
(SEQ ID NO: 17)
(GVGVPGVGVPGAGVPGVGVPGVGVP)ₘ;
or
b.
(SEQ ID NO: 18)
(GVGVPGVGVPGLGVPGVGVPGVGVP)ₘ;
wherein m is an integer between 2 and 32, inclusive of endpoints.

10. The fusion protein of claim 1, wherein the polypeptide with phase behavior comprises an amino acid sequence selected from:
a. (GVGVPGVGVPGAGVPGVGVPGVGVP)ₘ (SEQ ID NO: 19), wherein m is 8 or 16;
b. (GVGVPGAGVP)ₘ (SEQ ID NO: 20), wherein m is an integer between 5 and 80, inclusive of endpoints; or
c. (GXGVP)ₘ (SEQ ID NO: 21), wherein m is an integer between 10 and 160, inclusive of endpoints, and wherein X for each repeat is independently selected from the group consisting of glycine, alanine, valine, isoleucine, leucine, phenylalanine, tyrosine, tryptophan, lysine, arginine, aspartic acid, glutamic acid, and serine.

11. The fusion protein of claim 1, wherein the polypeptide with phase behavior has identity to a polypeptide of any one of SEQ ID NOS: 1-60, 217, or 262-264.

12. (canceled)

13. The fusion protein of claim 1, wherein the polypeptide with phase behavior has at least 95% identity to a polypeptide of SEQ ID NO: 56.

14. The fusion protein of claim 1, wherein the fusion protein has at least 95% identity to a polypeptide of SEQ ID NO: 90 and at least 95% identity to a polypeptide of any one of SEQ ID NOs: 56 and 262-264.

15. The fusion protein of claim 1, wherein the fusion protein has at least 95% identity to a polypeptide of SEQ ID NO: 90 and at least 95% identity to a polypeptide of SEQ ID NO: 56.

16. The fusion protein of claim 1, comprising a linker.

17. The fusion protein of claim 16, wherein the linker has an amino acid sequence with at least 95% identity to a polypeptide of any one of SEQ ID NOS: 143-216, or 261.

18. (canceled)

19. The fusion protein of claim 1, wherein the fusion protein has an amino acid sequence with at least 95% identity to a polypeptide of any one of SEQ ID NOs: 225 or 226.

20. The fusion protein of claim 1, wherein the fusion protein has at least 95% identity to a polypeptide of SEQ ID NO: 225 or 226.

21. The fusion protein of claim 1, wherein the Bs-CspB binds to RNA, DNA, or both.

22. (canceled)

23. (canceled)

24. (canceled)

25. (canceled)

26. (canceled)

27. (canceled)

28. The fusion protein of claim 1, wherein the Bs-CspB binds to single stranded DNA, double stranded DNA, polyadenosine (polyA) DNA, Z-conformation DNA (Z-DNA), or a combination thereof.

29. A nucleic acid encoding the fusion protein of claim 1.

30. (canceled)

31. A vector encoding a fusion protein of claim 1.

32. (canceled)

33. (canceled)

34. A method of purifying a single stranded nucleic acid comprising:
(i) contacting a composition comprising the single stranded nucleic acid and at least one contaminant with a fusion protein of claim 1, wherein the fusion protein binds to the single stranded nucleic acid to form a complex;
(ii) adding to the composition comprising the complex a first environmental factor, thereby increasing the size of the complex;
(iii) separating the complex from at least one contaminant; and
(iv) separating the single stranded nucleic acid from the fusion protein by contacting the complex with a second environmental factor, thereby forming a product comprising the single stranded nucleic acid.

35-53. (canceled)
